(19) United States
(12) Patent Application Publication
(10) Pub. No.: US 2025/0224670 A1
(43) Pub. Date: Jul. 10, 2025

(54) RESIST COMPOSITION, RESIST PATTERN FORMING METHOD, AND COMPOUND

(71) Applicant: TOKYO OHKA KOGYO CO., LTD., Kawasaki-shi (JP)

(72) Inventors: Daichi TAKAKI, Kawasaki-shi (JP); Tsuyoshi NAKAMURA, Kawasaki-shi (JP); Takashi NAGAMINE, Kawasaki-shi (JP)

(21) Appl. No.: 18/853,547

(22) PCT Filed: Apr. 11, 2023
(86) PCT No.: PCT/JP2023/014666

§ 371 Date: Oct. 2, 2024

(30) Foreign Application Priority Data
Apr. 12, 2022 (JP) 2022-065655

(51) Int. Cl. G03F 7/004; G03F 7/20

(52) U.S. Cl. CPC G03F 7/0045; G03F 7/2012

(57) ABSTRACT

A resist composition which generates an acid upon light exposure and whose solubility in a developing solution is changed by an action of the acid, in which the resist composition contains a compound represented by General Formula (d0) in which R^{Ar} represents an aromatic hydrocarbon group; t represents an integer of 1 or greater; R⁰¹ and R⁰² represent a chain hydrocarbon group; R⁰³ represents a chain hydrocarbon group or a hydrogen atom; or two or more of R⁰¹, R⁰², and R⁰³ are bonded to each other to form a ring structure; m represents an integer of 1 or greater; and M^{m+} represents an m-valent organic cation

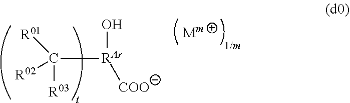

### TECHNICAL FIELD

The present invention relates to a resist composition, a resist pattern forming method, and a novel compound.

Priority is claimed on Japanese Patent Application No. 2022-065655, filed Apr. 12, 2022, the content of which is incorporated herein by reference.

### BACKGROUND ART

In recent years, in the manufacture of semiconductor elements and liquid crystal display elements, advances in lithography technologies have led to rapid progress in the field of pattern miniaturization. These pattern miniaturization techniques typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed by an action of an acid and an acid generator component that generates an acid upon light exposure has been used.

In the chemically amplified resist composition, a resin having a plurality of constitutional units is typically used in order to improve lithography characteristics and the like.

Further, in the resist pattern formation, the behavior of an acid generated by an acid generator component upon light exposure serves as an element that greatly affects the lithography characteristics. Meanwhile, a chemically amplified resist composition in which an acid generator component and an acid diffusion control agent that controls the diffusion of the acid generated from the acid generator component upon light exposure are used in combination has been suggested.

As one of techniques for improving resolution, a lithography method, which is so-called liquid immersion lithography (hereinafter, also referred to as “liquid immersion exposure”), in which a liquid (liquid immersion medium) having a refractive index higher than that of air is interposed between an objective lens of an exposure device and a sample to carry out light exposure (immersion exposure) is known.

According to the liquid immersion exposure, even in a case of using a light source with the same exposure wavelength, the same high resolution as in a case of using a light source with a shorter wavelength or a high NA lens can be achieved, and the depth of focus is not even degraded. Further, the liquid immersion exposure can be performed using an exposure apparatus of the related art. Therefore, liquid immersion exposure has been used in recent years because of realization of a resist pattern formation with high resolution and an excellent depth of focus at a low cost. The liquid immersion exposure is considered to be effective in forming any pattern shape and to be combined with a super-resolution technique such as a phase shift method or a deformed illumination method.

Recently, a technique using an ArF excimer laser as a light source is actively researched as the liquid immersion exposure technique. As the liquid immersion medium, water is mainly examined.

In the above-described liquid immersion exposure, them is a demand for a resist material having characteristics corresponding to the liquid immersion exposure technique in addition to typical lithography characteristics (sensitivity, resolution, roughness characteristics, and the like).

For example, in the liquid immersion exposure, in a case where a resist film is brought into contact with a liquid immersion solvent, elution of a substance in the resist film into the liquid immersion solvent (substance elution) occurs. The substance elution causes phenomena such as deterioration of a resist layer and a change in refractive index of a liquid immersion solvent, and is a factor in degradation of lithography characteristics and occurrence of defects (surface defects).

Here, “defects” refers to all defects detected in a case where a resist pattern after development is observed from just above the surface by, for example, a surface defect observation device (trade name “KLA”, manufactured by KLA Corporation). These defects refer to, for example, a defect due to adhesion of foreign substances or precipitates on the surface of the resist pattern after development, such as scum (resist residue), bubbles, and dust, a bridge between line patterns, a defect related to a pattern shape such as filling of pores in the contact hole pattern, and color unevenness of the pattern.

The amount of the substance to be eluted is affected by the characteristics (hydrophilicity, hydrophobicity, and the like) of the surface of the resist film. Therefore, in the resist composition, for example, the lithography characteristics are improved by increasing hydrophobicity of the surface of a resist film to suppress substance elution.

In the related art, it has been proposed that a compound having a fluorine atom is added to a resist composition used for such liquid immersion exposure. For example, Patent Document 1 discloses a resist composition in which a specific acid generator component having a fluorine atom, a fluorine-containing polymer compound that has a constitutional unit having a fluorine atom, and a base component are used in combination. Examples of the base component include a resist composition formed of a photodecomposable base that is decomposed upon light exposure and loses acid diffusion controllability.

### CITATION LIST

### Patent Document

Patent Document 1: Japanese Unexamined Patent Application, First Publication No. 2021-004992

### SUMMARY OF INVENTION

### Technical Problem

With the further pattern fining, the thickness of the resist film has been reduced, and resist materials of the related art are further required to be improved in various lithography characteristics such as high sensitivity in a case of the resist pattern formation and reduction of line width roughness (LWR: non-uniformity of the line width) in a case of a line pattern. In addition, in the resist pattern formation by the lithography method for performing immersion exposure, defects are more likely to occur, and the influence of substance elution on the liquid immersion solvent has been greater than before.

The present invention has been made in consideration of the above-described circumstances, and an object thereof is to provide a resist composition in which both roughness characteristics and an effect of suppressing occurrence of defects are enhanced, a resist pattern forming method using the resist composition, and a compound which is useful as a base component for the resist composition.

### Solution to Problem

In order to achieve the above-described object, the present invention employs the following configurations.

That is, according to a first aspect of the present invention, there is provided a resist composition which generates an acid upon light exposure and whose solubility in a developing solution is changed by an action of the acid, the resist composition including: a base material component (A) whose solubility in a developing solution is changed by the action of the acid; and a compound (D0) represented by General Formula (d0).

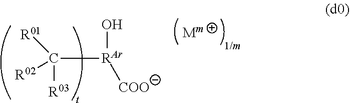

[In the formula. R^{Ar} represents a (t+2)-valent aromatic hydrocarbon group which may have a substituent. t represents an integer of 1 or greater. R⁰¹ and R⁰² each independently represent a chain-like hydrocarbon group, R⁰³ represents a chain-like hydrocarbon group or a hydrogen atom, or two or more of R⁰¹, R⁰², and R⁰³ are bonded to each other to form a ring structure. Here, in a case where two of R⁰¹, R⁰², and R⁰³ are bonded to each other to form a ring structure, the ring structure is an alicyclic ring, and the remaining one represents a chain-like hydrocarbon group or a hydrogen atom. The chain-like hydrocarbon group may have a substituent. The ring structure may have a substituent. m represents an integer of 1 or greater, and M^{m+} represents an m-valent organic cation.]

According to a second aspect of the present invention, there is provided a resist pattern forming method including: a step of forming a resist film on a support using the resist composition according to the first aspect; a step of performing liquid immersion exposure on the resist film; and a step of developing the resist film exposed to light to form a resist pattern.

According to a third aspect of the present invention, there is provided a compound which is represented by General Formula (d0).

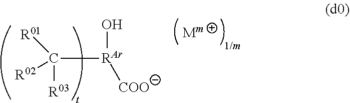

[In the formula, R^{Ar} represents a (t+2)-valent aromatic hydrocarbon group which may have a substituent. t represents an integer of 1 or greater. R⁰¹ and R⁰² each independently represent a chain-like hydrocarbon group, R⁰³ represents a chain-like hydrocarbon group or a hydrogen atom, or two or more of R⁰¹, R⁰², and R⁰³ are bonded to each other to form a ring structure. Here, in a case where two of R⁰¹, R⁰², and R⁰³ are bonded to each other to form a ring structure, the ring structure is an alicyclic ring, and the remaining one represents a chain-like hydrocarbon group or a hydrogen atom. The chain-like hydrocarbon group may have a substituent. The ring structure may have a substituent. m represents an integer of 1 or greater, and M^{m+} represents an m-valent organic cation.]

### Advantageous Effects of Invention

According to the present invention, it is possible to provide a resist composition in which both roughness characteristics and an effect of suppressing occurrence of defects are enhanced, a resist pattern forming method using the resist composition, and a compound which is useful as a base component for the resist composition.

### DESCRIPTION OF EMBODIMENTS

In the present specification and the scope of the present claims, the term “aliphatic” is a relative concept used in relation to the term “aromatic”, and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “base material component” denotes an organic compound having a film-forming ability. Organic compounds used as the base material component are classified into non-polymers and polymers. As the non-polymers, those having a molecular weight of 500 or greater and less than 4000 are typically used. Hereinafter, the term “low-molecular-weight compound” denotes a non-polymer having a molecular weight of 500 or greater and less than 4000. As the polymer, those having a molecular weight of 1000 or greater are typically used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, the acrylic acid ester includes itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

Such a resist composition contains a base material component (A) (hereinafter, also referred to as “component (A)”) whose solubility in a developing solution is changed by an action of an acid, and a compound (D0) (hereinafter, also referred to as “component (D0)”) represented by General Formula (d0).

In a case where a resist film is formed using the resist composition according to the present embodiment and the formed resist film is subjected to selective exposure, an acid is generated at exposed portions of the resist film, and the generated acid acts on the component (A) to change the solubility of the component (A) in a developing solution. On the other hand, the solubility of the component (A) in a developing solution is not changed at unexposed portions of the resist film, which generates the difference in solubility in the developing solution between exposed portions and unexposed portions of the resist film. Therefore, in a case where the resist film is developed, the exposed portion of the resist film is dissolved and removed to form a positive-tone resist pattern in a case where the resist composition is of a positive tone, whereas the unexposed portion of the resist film is dissolved and removed to form a negative-tone resist pattern in a case where the resist composition is of a negative tone.

In the present specification, a resist composition which forms a positive-tone resist pattern by dissolving and removing the exposed portion of the resist film is referred to as a positive-tone resist composition, and a resist composition which forms a negative-tone resist pattern by dissolving and removing the unexposed portion of the resist film is referred to as a negative-tone resist composition. The resist composition of the present embodiment may be a positive-tone resist composition or a negative-tone resist composition. Further, the resist composition of the present embodiment may be used in an alkali developing process using an alkali developing solution in the developing treatment in a case of forming a resist pattern or may be used in a solvent developing process using a developing solution containing an organic solvent (organic developing solution) in the developing treatment.

### <Component (A)>

In the resist composition of the present embodiment, it is preferable that the component (A) has a resin component (A1) whose solubility in a developing solution is changed by the action of an acid (hereinafter, also referred to as “component (A1)”). Since the polarity of the base material component before and after the light exposure is changed by using the component (A1), an excellent development contrast can be obtained not only in an alkali developing process but also in a solvent developing process.

As the component (A), it is preferable to use at least the component (A1), and other polymer compounds and/or low-molecular-weight compounds may be used in combination with the component (A1).

In a case of applying an alkali developing process, a base material component containing the component (A1) is substantially insoluble in an alkali developing solution prior to exposure, but in a case where an acid is generated upon light exposure, the action of this acid causes an increase in the polarity of the base material component, thereby increasing the solubility of the base material component in an alkali developing solution. Therefore, in the resist pattern formation, in a case where a resist film obtained by coating a support with the resist composition is selectively exposed to light, the exposed portion of the resist film changes from being insoluble in an alkali developing solution to being soluble in an alkali developing solution, while the unexposed portion of the resist film does not change and remains insoluble in an alkali developing solution. Therefore, a contrast can be obtained between the exposed portion and the unexposed portion of the resist film by performing development with an alkali so that a positive-tone resist pattern is formed.

Meanwhile, in a case of a solvent developing process, the base material component containing the component (A1) exhibits high solubility in an organic developing solution prior to exposure, and in a case where an acid is generated upon light exposure, the polarity of the component (A1) is increased by the action of the generated acid, thereby decreasing the solubility of the component (A1) in an organic developing solution. Therefore, in the resist pattern formation, in a case where a resist film obtained by coating a support with the resist composition is selectively exposed to light, the exposed portion of the resist film changes from being soluble in an organic developing solution to being insoluble in an organic developing solution, while the unexposed portion of the resist film does not change and remains soluble in an organic developing solution. Therefore, a contrast can be obtained between the exposed portion and the unexposed portion of the resist film by performing development with an organic developing solution so that a negative-tone resist pattern is formed.

In the resist composition according to the present embodiment, the component (A) may be used alone or in a combination of two or more kinds thereof.

### In Regard to Component (A1)

The component (A1) is a resin component whose solubility in a developing solution is changed by the action of an acid.

As the component (A1), those having a constitutional unit (a1) containing an acid decomposable group whose polarity is increased by the action of an acid are preferable.

The component (A1) may have other constitutional units as necessary in addition to the constitutional unit (a1).

<<Constitutional Unit (a1)>>

The constitutional unit (a1) is a constitutional unit that contains an acid decomposable group whose polarity is increased by the action of an acid.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group. Examples of the polar group include a carboxy group, a hydroxyl group, an amino group, and a sulfo group (—SO₃H).

More specific examples of the acid decomposable group include a group in which the above-described polar group has been protected with an acid dissociable group (such as a group in which a hydrogen atom of the OH-containing polar group has been protected with an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

Examples of the acid dissociable group are the same as those which have been suggested as the acid dissociable groups of the base resin for a chemically amplified resist composition.

Specific examples of the acid dissociable group of the base resin suggested for a chemically amplified resist composition include “acetal type acid dissociable group”, “tertiary alkyl ester type acid dissociable group”, and “tertiary alkyloxycarbonyl acid dissociable group” described below.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

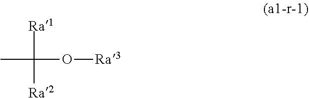

[in the formula. Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ or Ra′² represents a hydrogen atom and more preferable that both Ra′¹ and Ra′² represent a hydrogen atom.

In a case where Ra′¹ or Ra′² represents an alkyl group, examples of the alkyl group include the same alkyl groups exemplified as the substituent which may be bonded to the carbon atom at the α-position in the description of the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of a single kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}-]decanyl group, a tricyclo[3.3.1.1^{3,7}]decanyl group, a tetracyclo[6.2.1.1^{3,6}.0^{2,7}]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience.

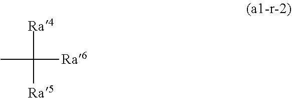

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ independently represent a hydrocarbon group without being bonded to one another, suitable examples thereof include a group represented by General Formula (a1-r2-4).

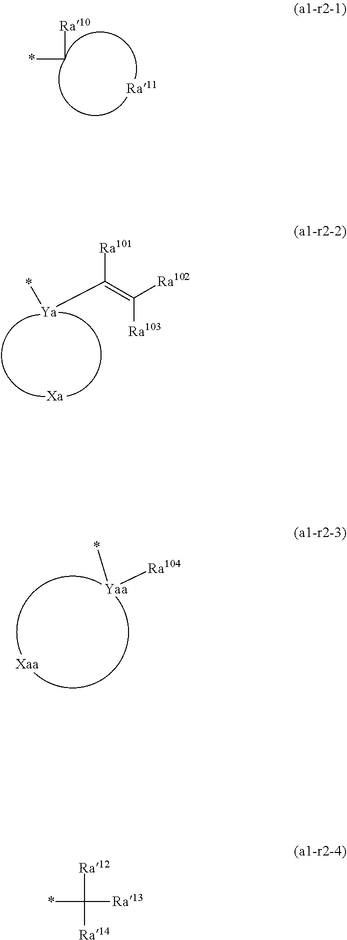

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹¹ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a chain-like monovalent saturated hydrocarbon group having 1 to 10 carbon atoms. Some or all hydrogen atoms in this chain-like saturated hydrocarbon group may be substituted. Ra′¹⁴ represents a hydrocarbon group which may have a substituent. * represents a bonding site.]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups exemplified as the aliphatic hydrocarbon group (alicyclic hydrocarbon group) which is a monocyclic group or a polycyclic group as Ra′³ in Formula (a1-r-1). Among these, a monocyclic alicyclic hydrocarbon group is preferable, specifically, a cyclopentyl group or a cyclohexyl group is more preferable, and a cyclopentyl group is still more preferable.

In Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in Formula (a1-r-1).

The cyclic hydrocarbon group that is formed by Xa together with Ya may have a substituent. Examples of the substituent include those exemplified as the substituents that the cyclic hydrocarbon group as Ra′³ may have.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}]decanyl group, a tricyclo[3.3.1.1^{3,7}]decanyl group, a tetracyclo[6.2.1.1^{3,6}.0^{2.7}] dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same substituents as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group exemplified as the aliphatic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, a propyl group, a hydroxy group, a carboxy group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a chain-like monovalent saturated hydrocarbon group having 1 to 10 carbon atoms. Examples of the chain-like monovalent saturated hydrocarbon group having 1 to 10 carbon atoms as Ra′¹² and Ra′¹³ include those exemplified as the chain-like monovalent saturated hydrocarbon group having 1 to 10 carbon atoms as Ra¹⁰¹ to Ra¹⁰³. Some or all hydrogen atoms in this chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent thereof include the same substituents as those for Ra^{x5}.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same groups as those for the substituent that Ra¹⁰⁴ may have.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

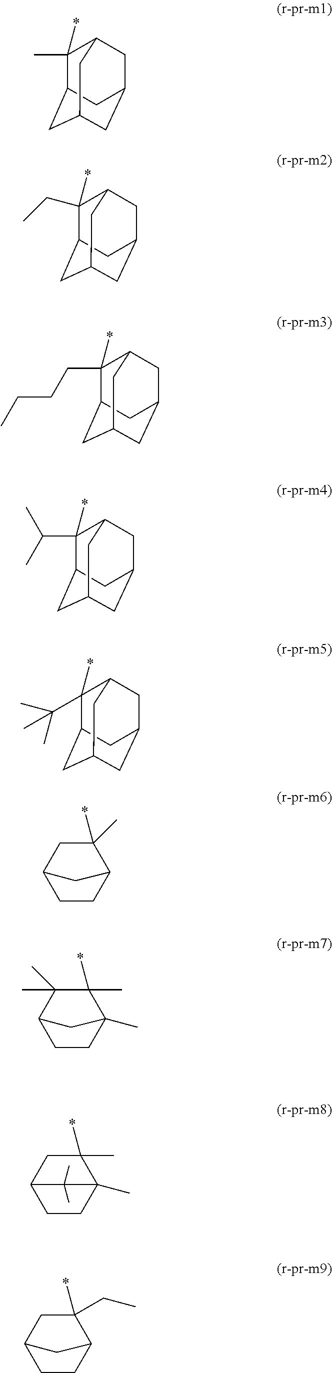

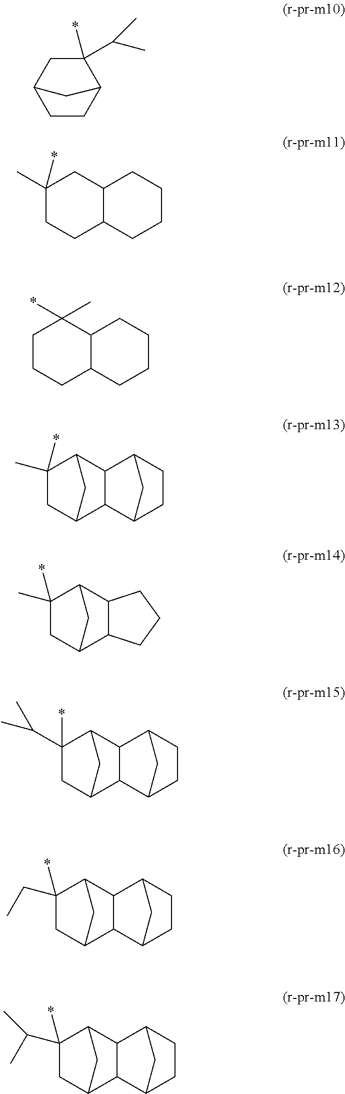

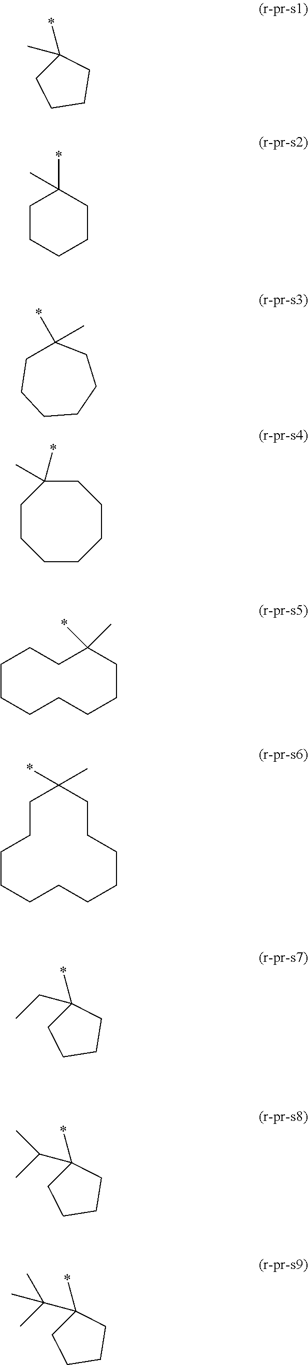

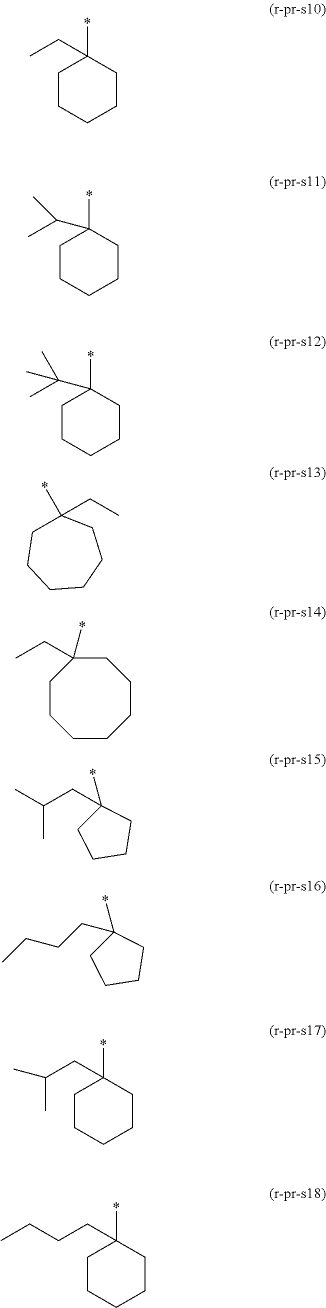

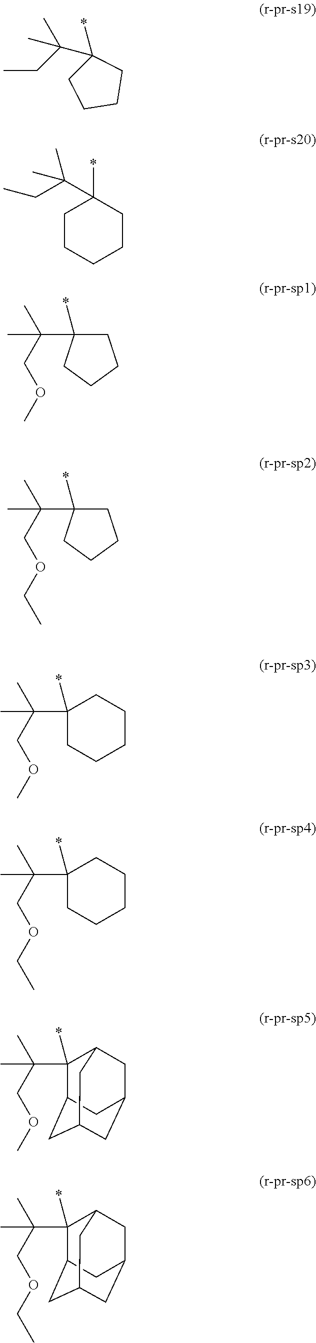

Specific examples of the group represented by Formula (a1-r2-2) are shown below.

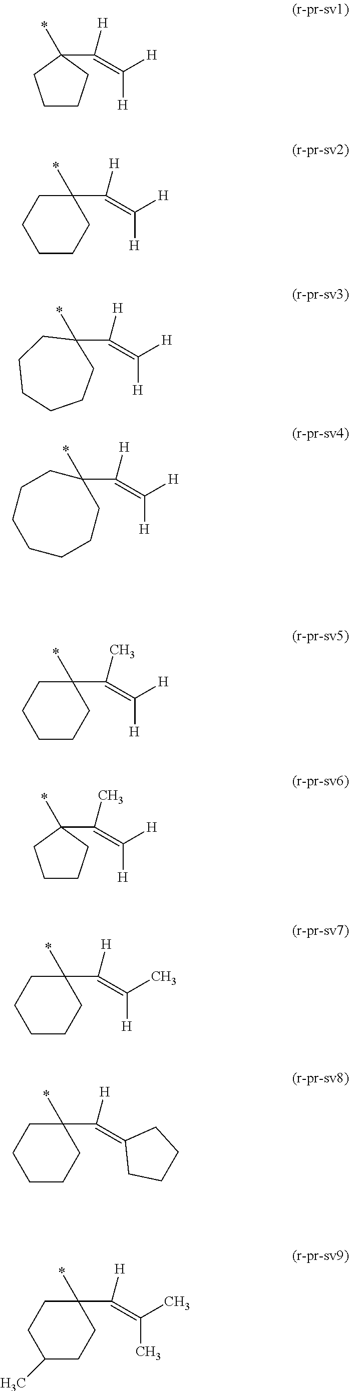

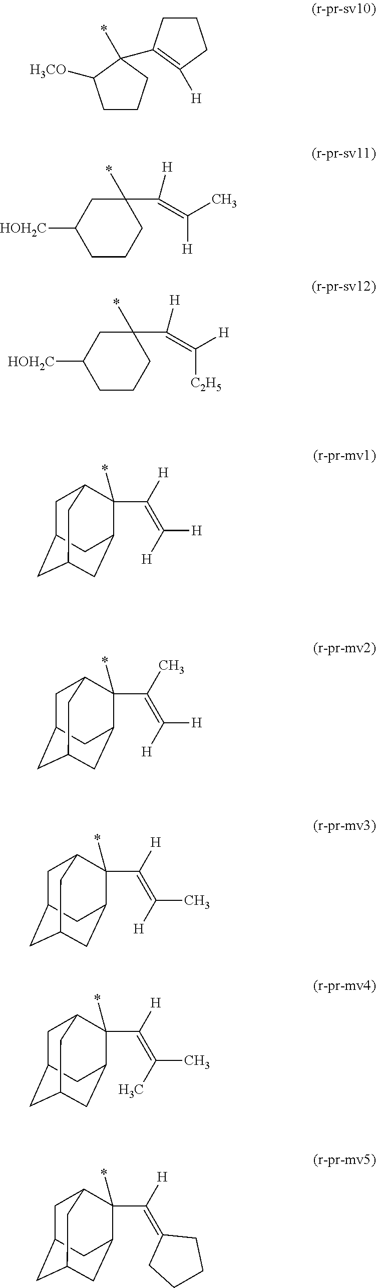

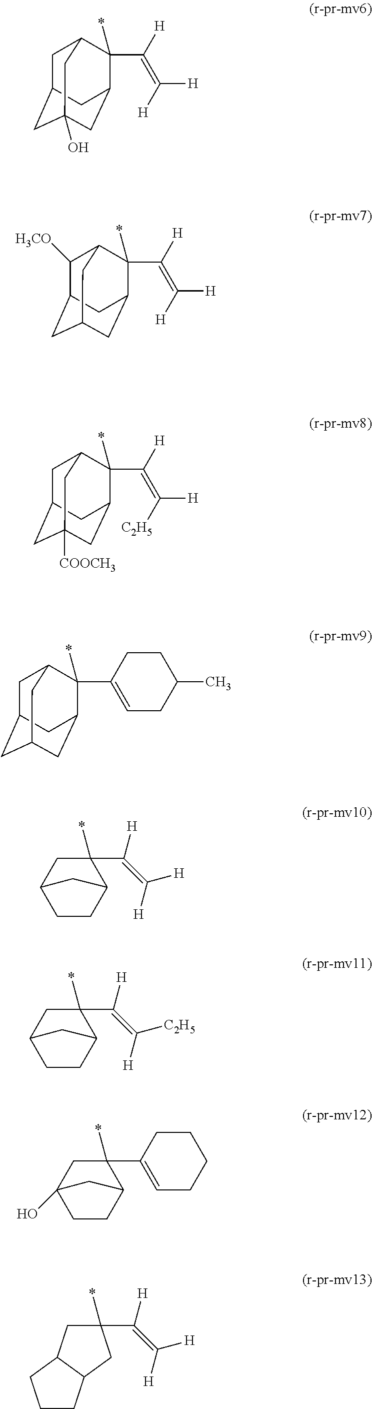

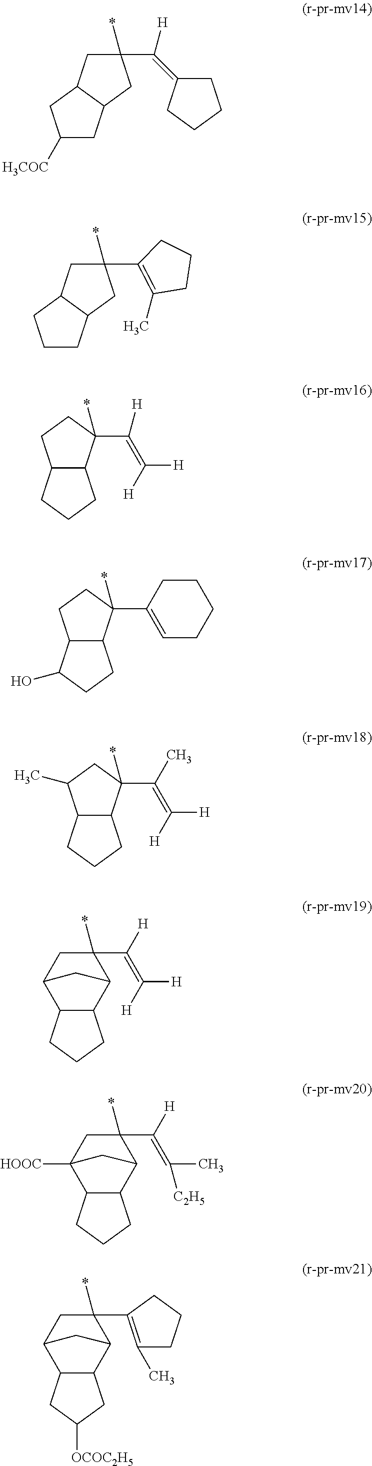

Specific examples of the group represented by Formula (a1-r2-3) are shown below.

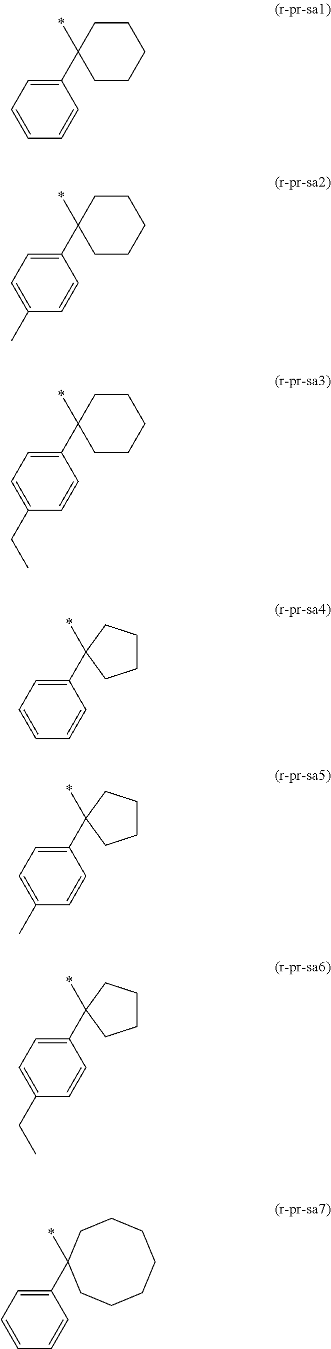

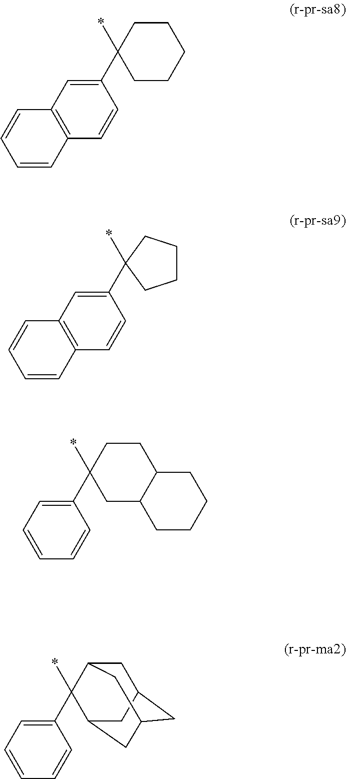 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

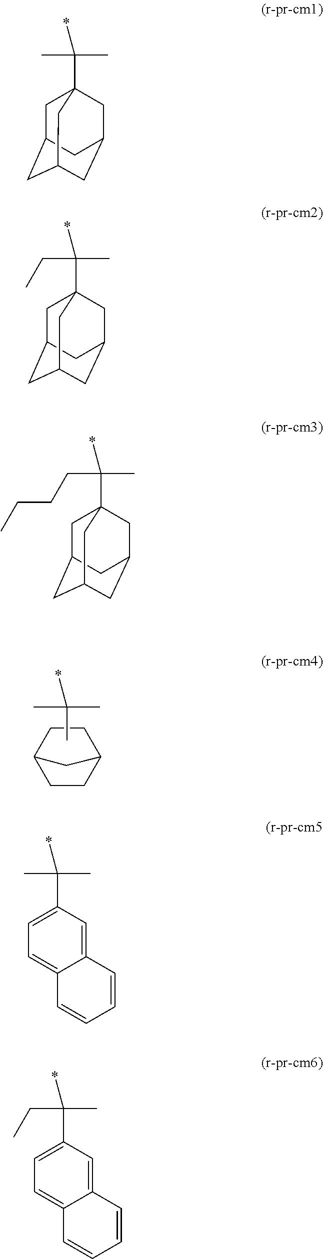

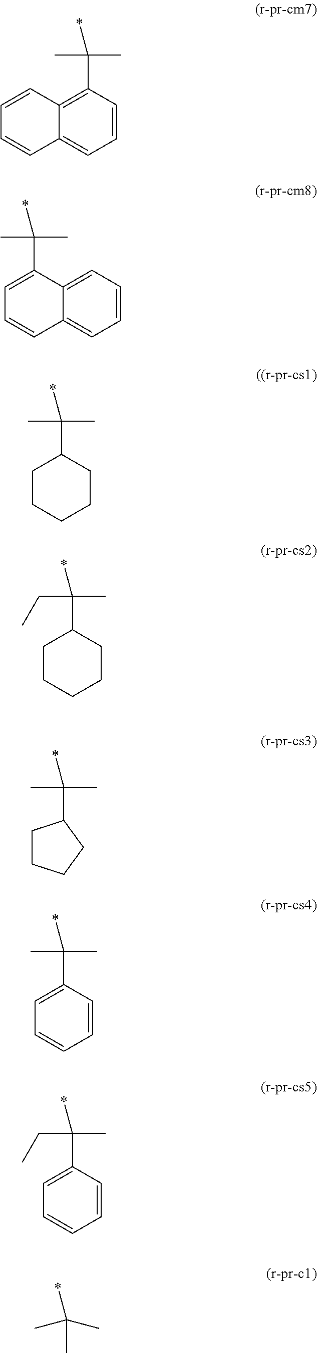

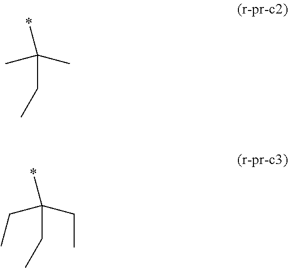

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

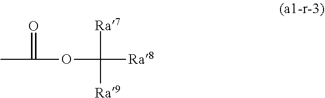

[In the formula, Ra′⁷ to Ra′⁹ each represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2) shown below.

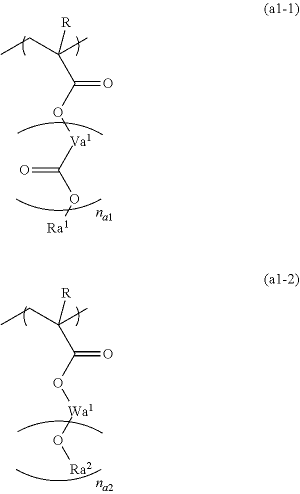

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid dissociable group represented by General Formula (a1-r-1) or (a1-r-2). Wa¹ represents a (nₐ₂+1)-valent hydrocarbon group, nₐ₂ represents an integer of 1 to 3, and Ra² represents an acid dissociable group represented by General Formula (a1-r-1) or (a1-r-3).]

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. The number of carbon atoms in the polycycloalkane is preferably in a range of 7 to 12, and specific examples thereof include adamantane, norbornane, isobornane, tricyclo[5.2.1.0^{2,6}]decane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by Formula (a1-r-1) or (a1-r-2).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent to tetravalent and more preferably divalent or trivalent.

In the Formula (a1-2), Ra² represents an acid dissociable group represented by General Formula (a1-r-1) or (a1-r-3).

Specific examples of the constitutional unit represented by Formula (a1-1) are shown below.

In the formulae shown below. R^{α} represents a hydrogen atom, a methyl group, or a trifluoromethyl group.

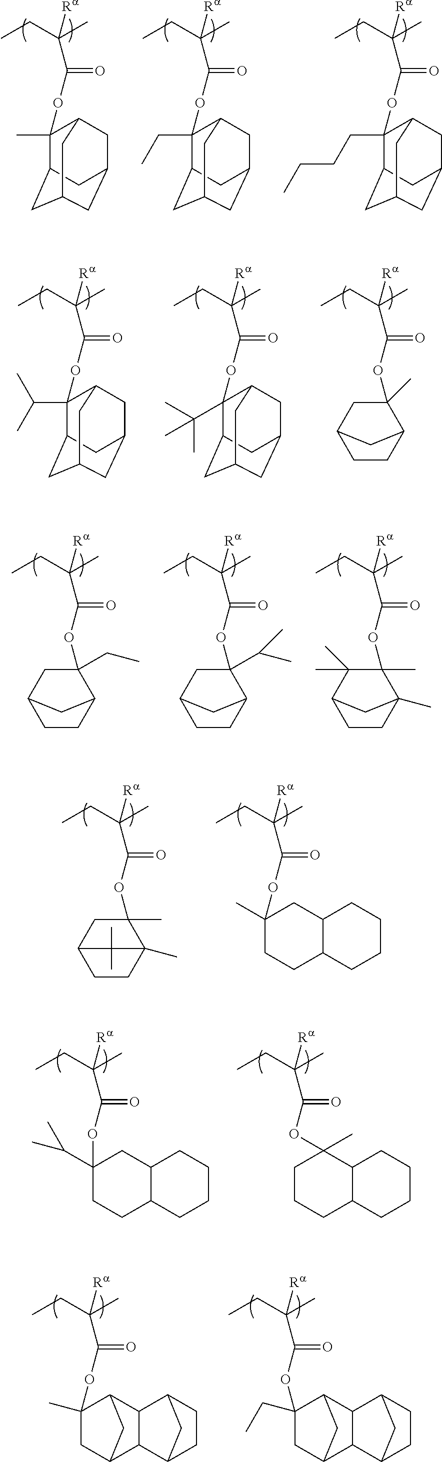

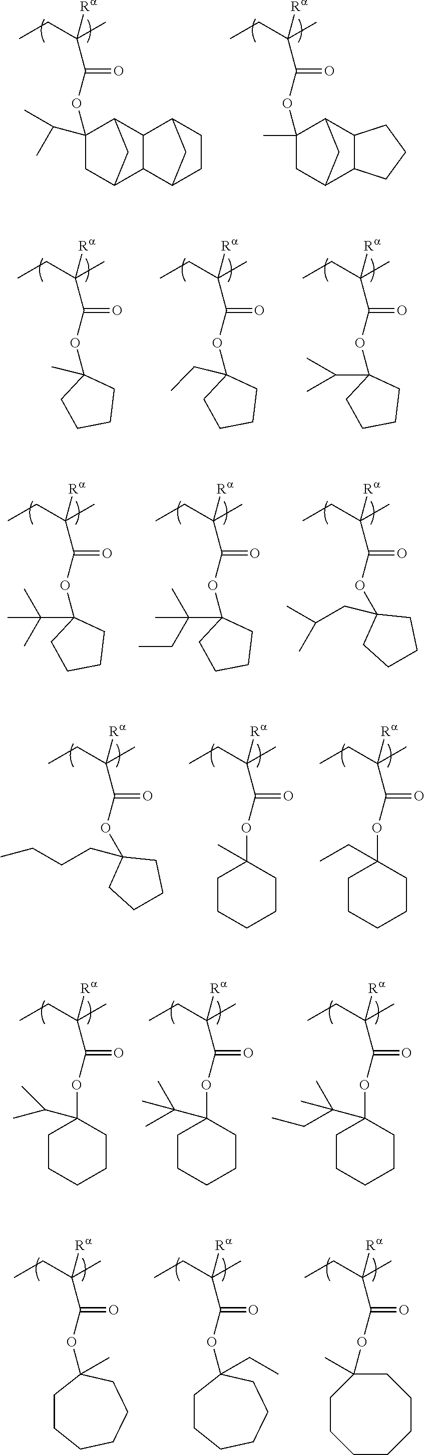

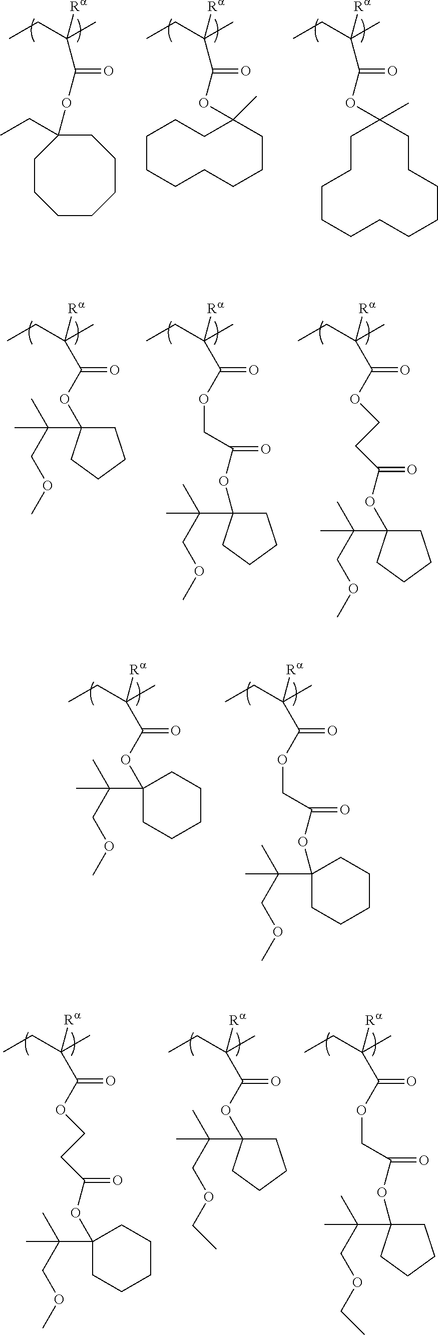

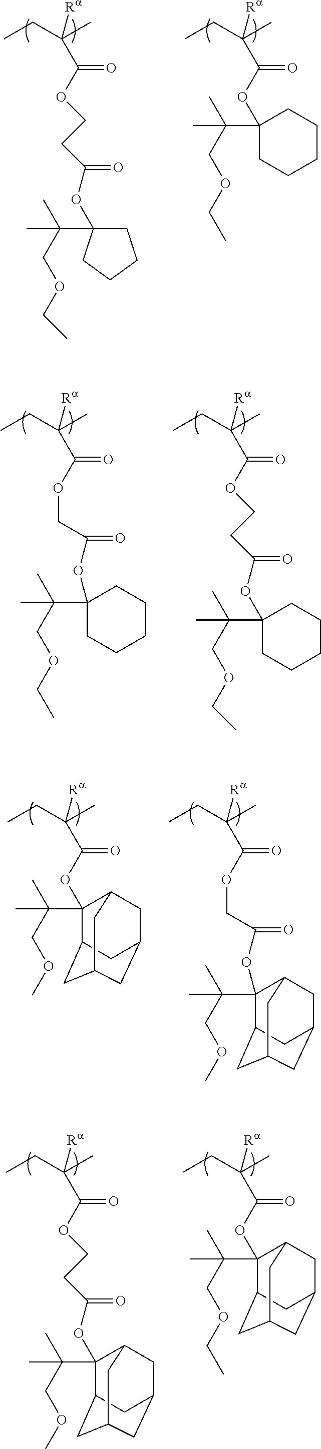

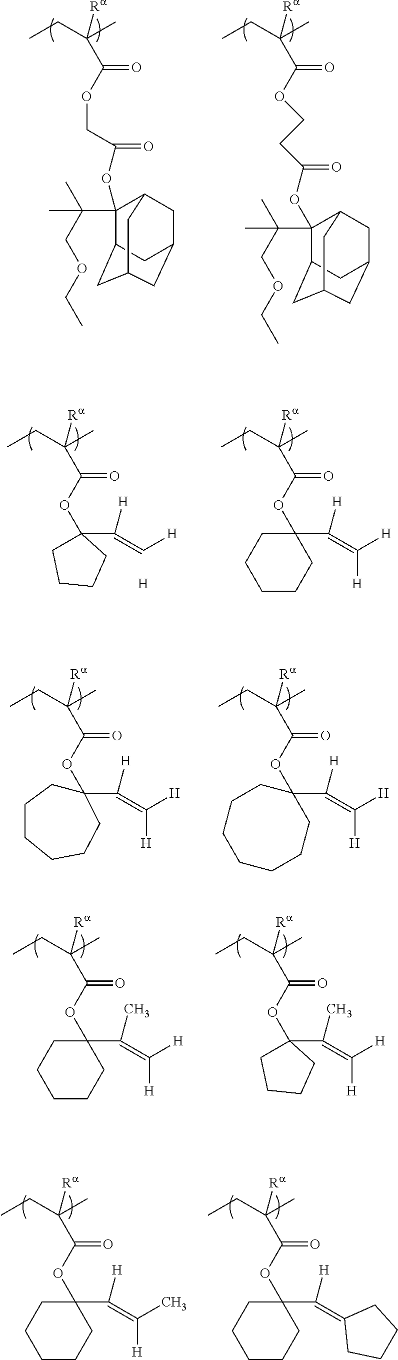

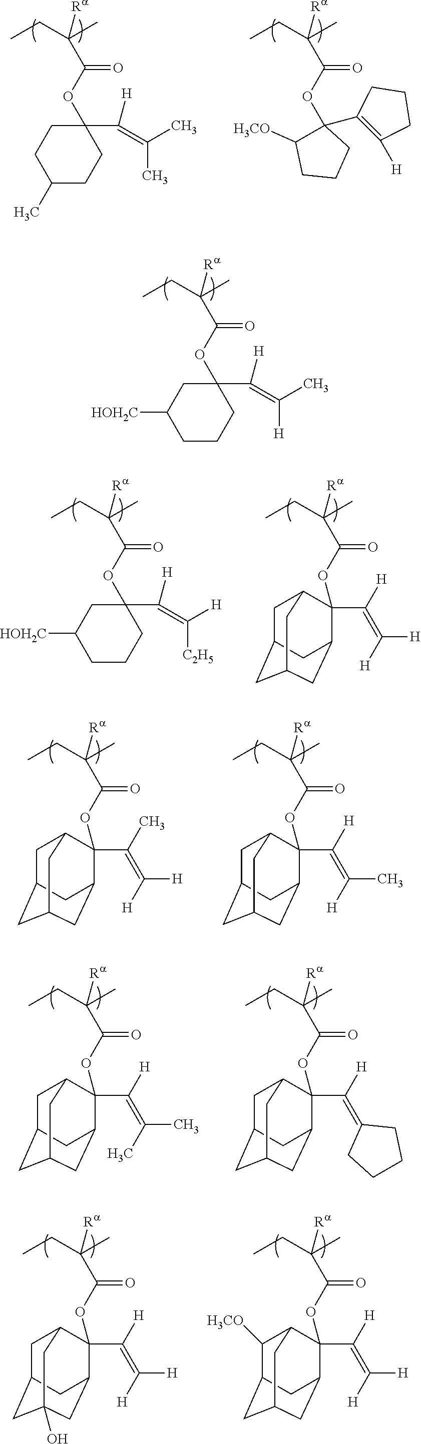

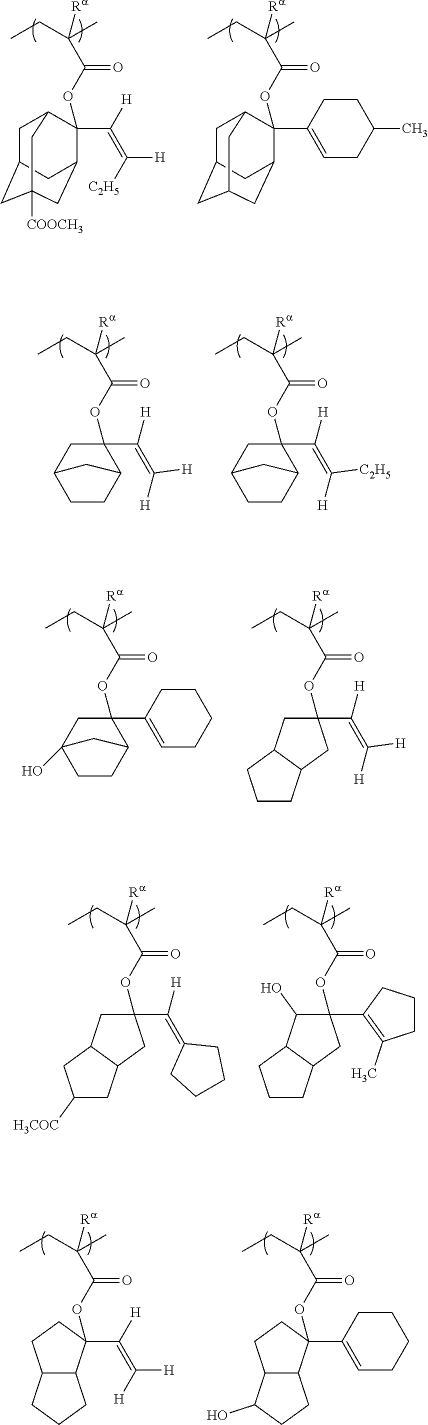

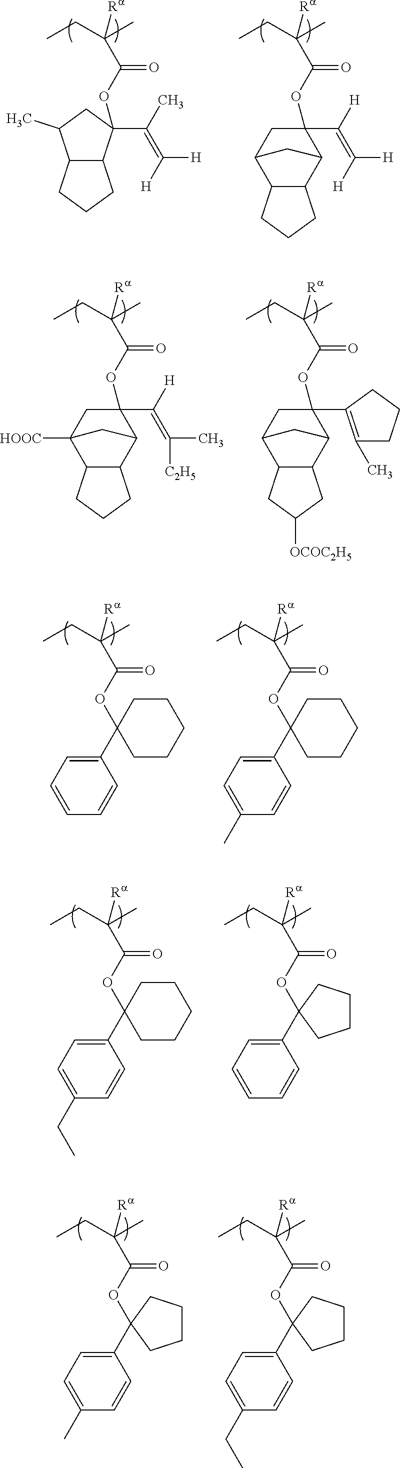

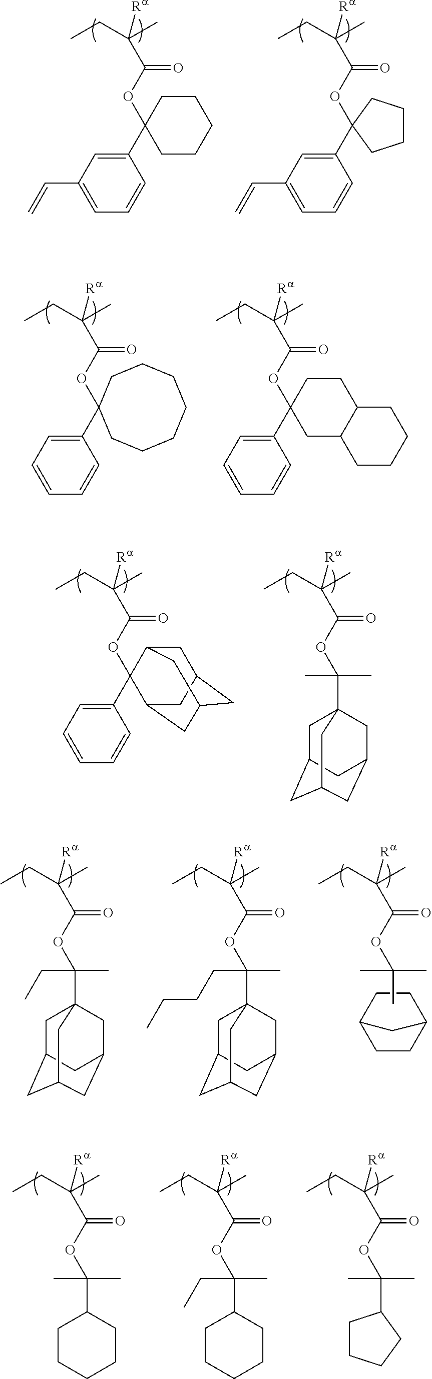

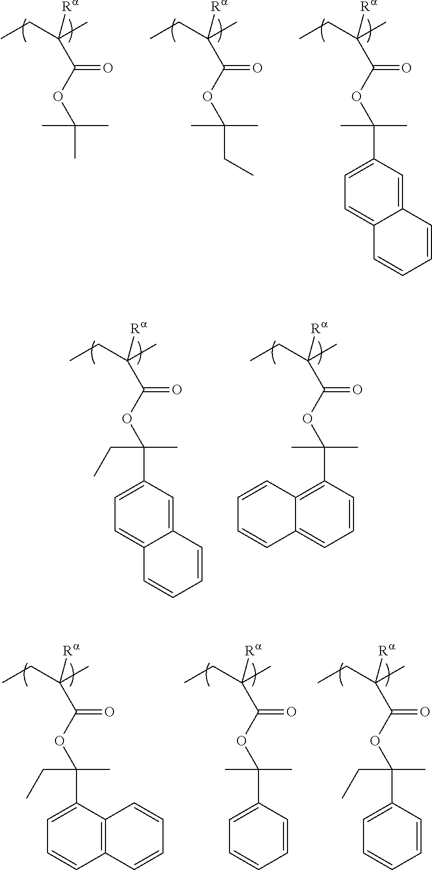

The constitutional unit (a1) included in the component (A1) may be used alone or two or more kinds thereof.

The constitutional unit (a1) is more preferably a constitutional unit represented by Formula (a1-1) from the viewpoint of easily enhancing lithography characteristics (the sensitivity, the shape, and the like).

Among the examples, as the constitutional unit (a1), those having a constitutional unit represented by General Formula (a1-1-1) are particularly preferable.

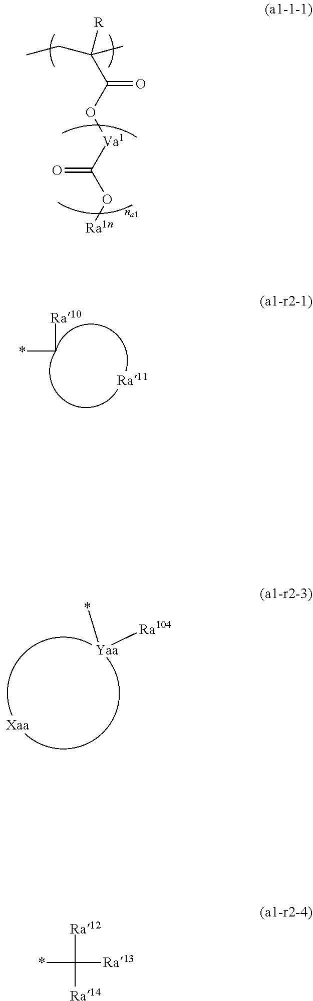

[In the formulae, Ra¹″ represents an acid dissociable group represented by General Formula (a1-r2-1), (a1-r2-3), or (a1-r2-4).]

R, Va¹, and nₐ₁ in Formula (a1-1-1) each have the same definition as that for R, Va¹, and nₐ₁ in Formula (a1-1).

The description of the acid dissociable group represented by General Formula (a1-r2-1), (a1-r2-3), or (a1-r2-4) is the same as described above. Among them, it is preferable to select a group in which the acid dissociable group is a cyclic group due to the fact that the reactivity can be increased, which is suitable.

In Formula (a1-1-1), it is preferable that Ra¹″ represents an acid dissociable group represented by General Formula (a1-r2-1) among the examples described above.

The proportion of the constitutional unit (a1) in the component (A1) is preferably in a range of 5% to 80% by mole, more preferably in a range of 10% to 75% by mole, still more preferably in a range of 30% to 70% by mole, and particularly preferably in a range of 40% to 70% by mole with respect to the total amount (100% by mole) of all constitutional units constituting the component (A1).

By setting the proportion of the constitutional unit (a1) to be greater than or equal to the lower limits of the above-described preferable ranges, lithography characteristics such as the sensitivity, the resolution, and improved roughness are improved. Further, in a case where the proportion of the constitutional unit (a1) is less than or equal to the upper limits of the above-described preferable ranges, the constitutional unit (a1) and other constitutional units can be balanced, and the lithography characteristics are improved.

### <<Other Constitutional Units>>

The component (A1) may have other constitutional units as necessary in addition to the constitutional unit (a1) described above.

Examples of other constitutional units include a constitutional unit (a2) that contains a lactone-containing cyclic group, a —SO₂-containing cyclic group, or a carbonate-containing cyclic group; a constitutional unit (a3) that contains a polar group-containing aliphatic hydrocarbon group; a constitutional unit (a4) that contains an acid non-dissociable aliphatic cyclic group; a constitutional unit (st) derived from styrene or a styrene derivative; a constitutional unit derived from a hydroxystyrene or a hydroxystyrene derivative; and a constitutional unit that generates an acid upon light exposure.

### In Regard to Constitutional Unit (a2):

The component (A1) may further have a constitutional unit (a2) (here, those corresponding to the constitutional unit (a1) are excluded) containing a lactone-containing cyclic group, a —SO₂-containing cyclic group, or a carbonate-containing cyclic group, in addition to the constitutional unit (a1).

In a case where the component (A1) is used for forming a resist film, the lactone-containing cyclic group, the —SO₂-containing cyclic group, or the carbonate-containing cyclic group in the constitutional unit (a2) is effective for improving the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

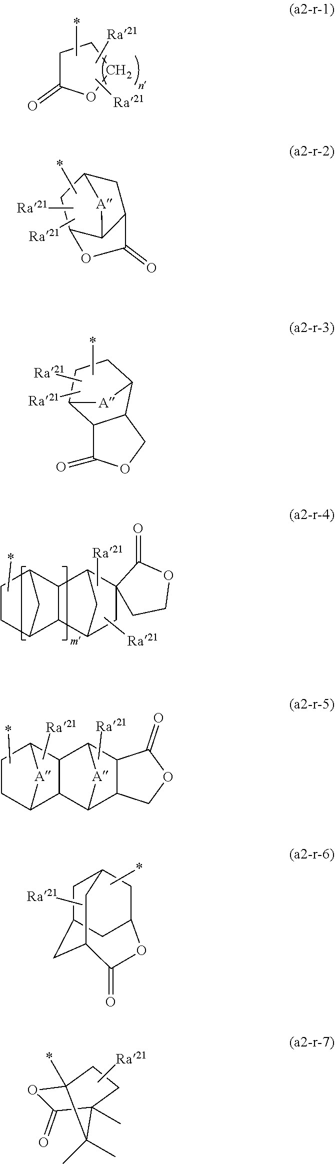

[in the formulae, each Ra′²¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or an —SO₂— containing cyclic group; A″ represents an oxygen atom, a sulfur atom, or an alkylene group having 1 to 5 carbon atoms, which may contain an oxygen atom (—O—) or a sulfur atom (—S—); and n′ represents an integer in a range of 0 to 2, and m′ is 0 or 1. * represents a bonding site.]

In General Formulae (a2-r-1) to (a2-r-7), it is preferable that the alkyl group as Ra′²¹ is an alkyl group having 1 to 6 carbon atoms. It is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group exemplified as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ are substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, a lactone-containing cyclic group, a carbonate-containing cyclic group, or a —SO₂-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclo[5.2.1.0^{2,6}]decane, or tetracyclododecane.

Examples of the lactone-containing cyclic group as R″ include the same groups as those for the groups each represented by General Formulae (a2-r-1) to (a2-r-7).

The carbonate-containing cyclic group as R″ has the same definition as that for the carbonate-containing cyclic group described below. Specific examples of the carbonate-containing cyclic group include groups each represented by General Formulae (ax3-r-1) to (ax3-r-3).

The —SO₂-containing cyclic group as R″ has the same definition as that for the —SO₂-containing cyclic group described below, and specific examples thereof include a group represented by any of General Formulae (a5-r-1) to (a5-r-4).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

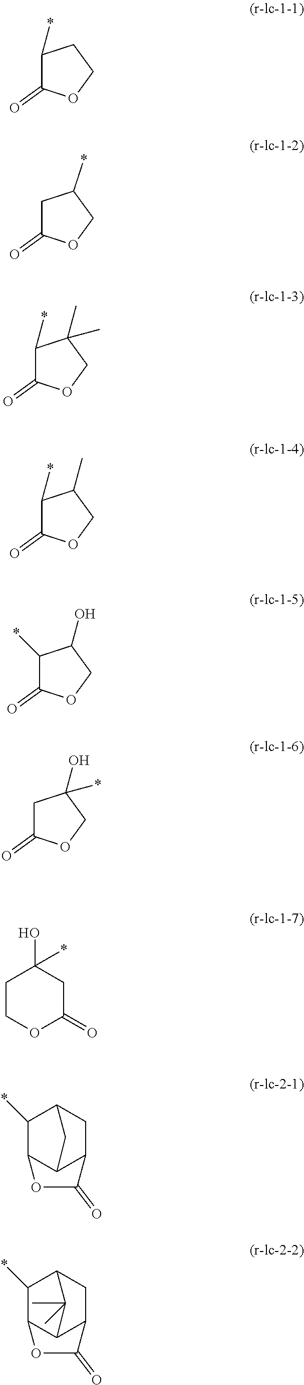

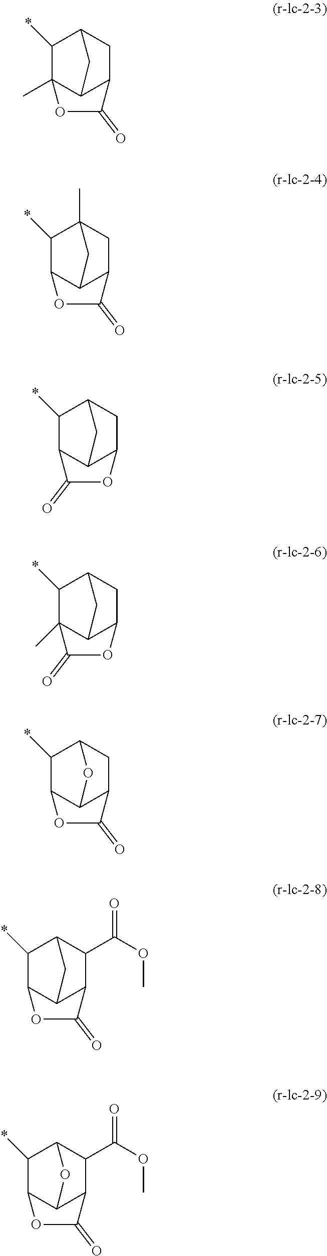

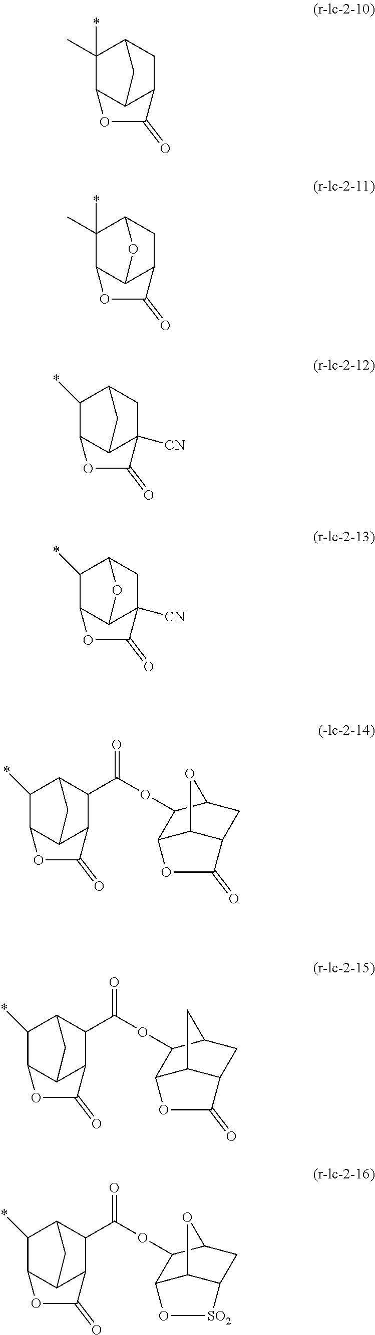

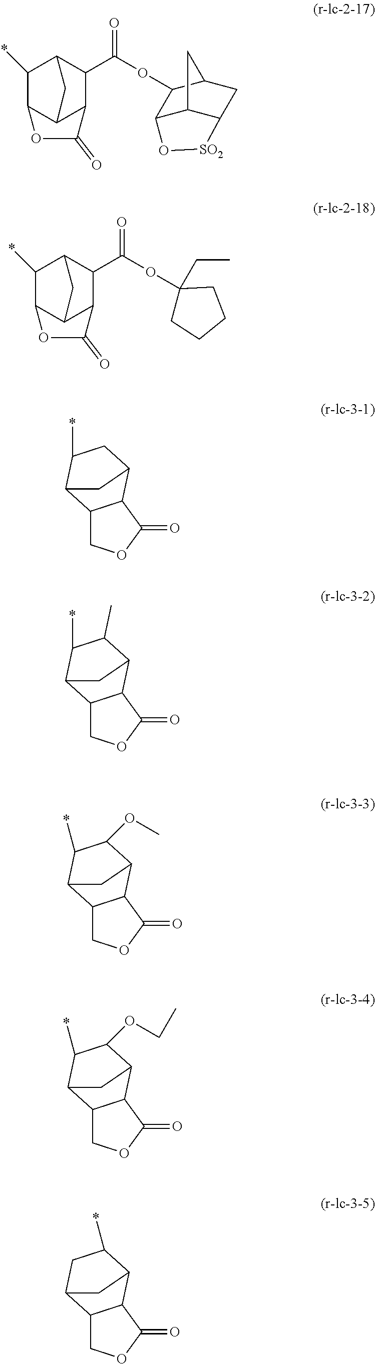

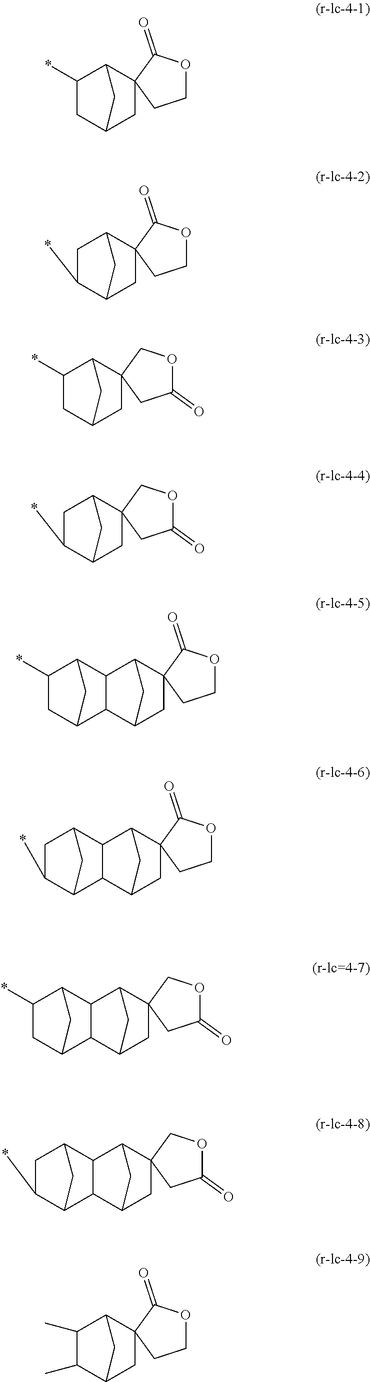

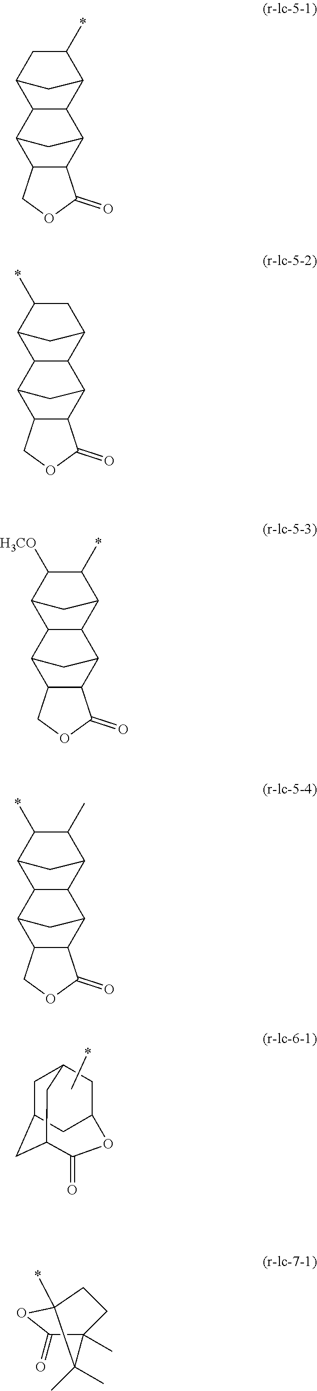

The term “—SO₂-containing cyclic group” denotes a cyclic group having a ring containing —SO₂— in the ring skeleton thereof. Specifically, the —SO₂-containing cyclic group is a cyclic group in which the sulfur atom (S) in —SO₂— forms a part of the ring skeleton of the cyclic group. In a case where the ring containing —SO₂— in the ring skeleton thereof is counted as the first ring and the group has only the ring, the group is referred to as a monocyclic group. In a case where the group further has other ring structures, the group is referred to as a polycyclic group regardless of the structures. The —SO₂-containing cyclic group may be a monocyclic group or a polycyclic group.

The —SO₂-containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof.

More specific examples of the —SO₂-containing cyclic group include groups each represented by General Formulae (a5-r-1) to (a5-r-4).

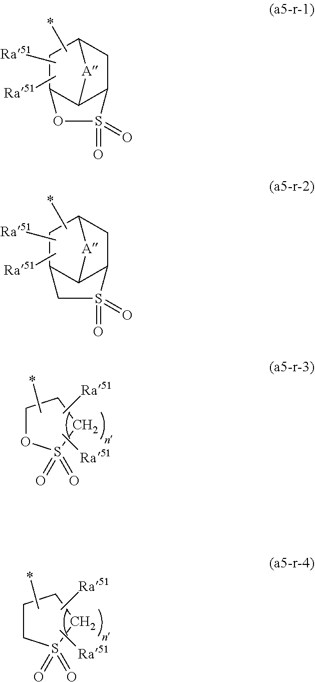

[In the formulae, Ra′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or a —SO₂—-containing cyclic group; A″ represents an oxygen atom, a sulfur atom, or an alkylene group having 1 to 5 carbon atoms, which may contain an oxygen atom or a sulfur atom; and n′ represents an integer in a range of 0 to 2. * represents a bonding site.]

In General Formulae (a5-r-1) and (a5-r-2), A″ has the same definition as that for A″ in General Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′⁵¹ include the same groups as those for Ra′²¹ in General Formulae (a2-r-1) to (a2-r-7).

Specific examples of the groups each represented by General Formulae (a5-r-1) to (a5-r-4) are shown below. In the formulae shown below, “Ac” represents an acetyl group.

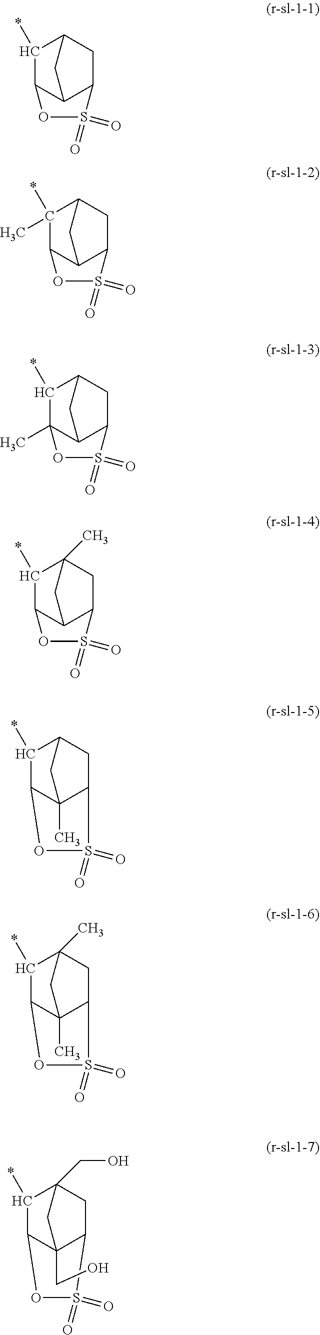

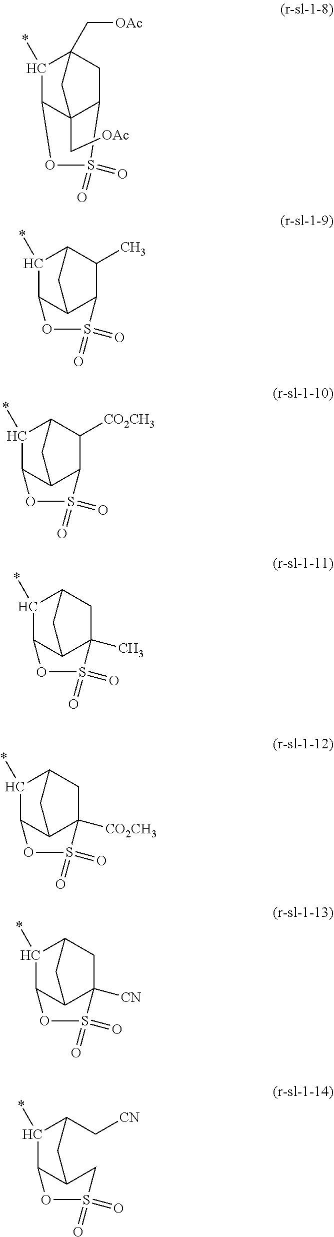

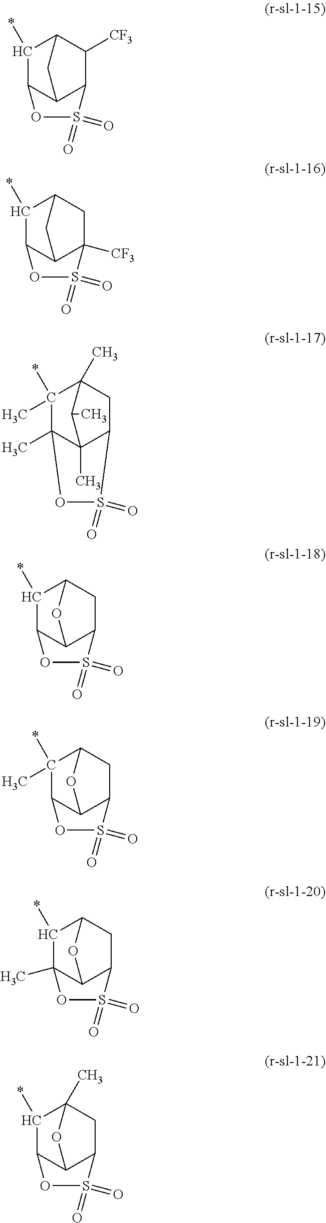

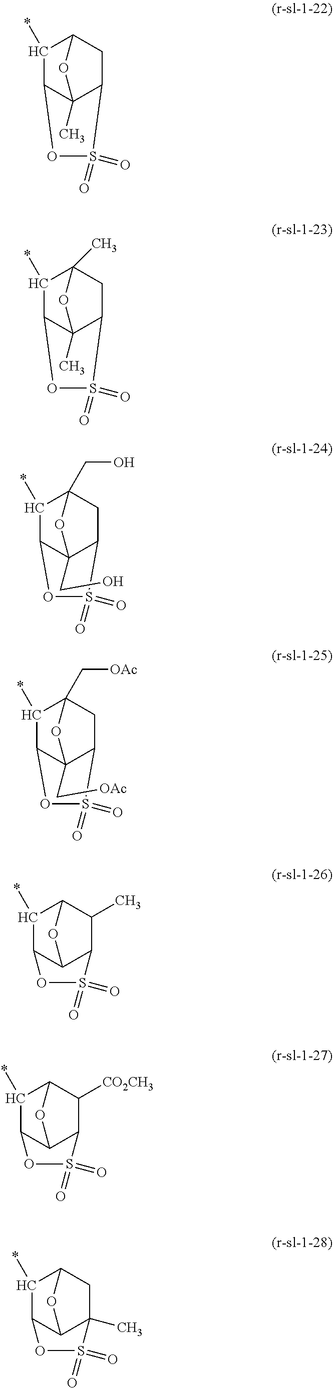

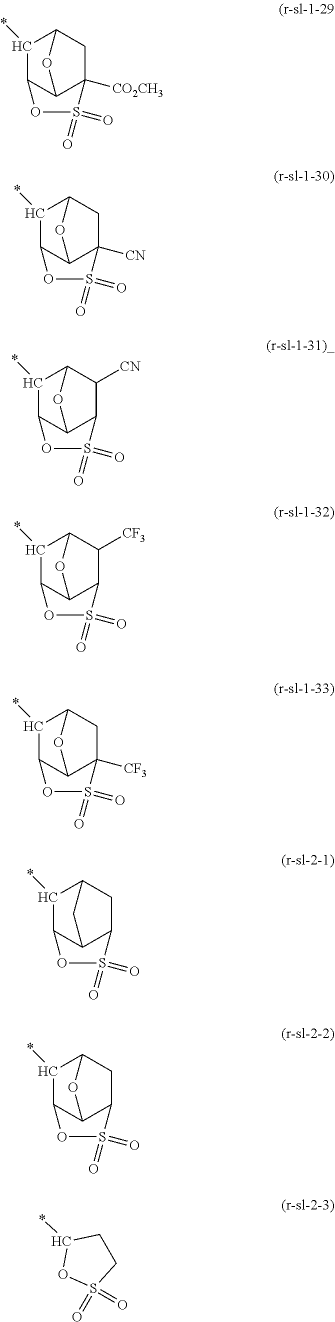

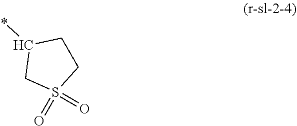

The term “carbonate-containing cyclic group” indicates a cyclic group that has a ring (a carbonate ring) containing —O—C(═O)—O— in the ring skeleton thereof. In a case where the carbonate ring is counted as the first ring and the group has only the carbonate ring, the group is referred to as a monocyclic group. Further, in a case where the group has other ring structures, the group is referred to as a polycyclic group regardless of the structures. The carbonate-containing cyclic group may be a monocyclic group or a polycyclic group.

The carbonate-containing cyclic group is not particularly limited, and an optional group can be used. Specific examples thereof include groups each represented by General Formulae (ax3-r-1) to (ax3-r-3) shown below.

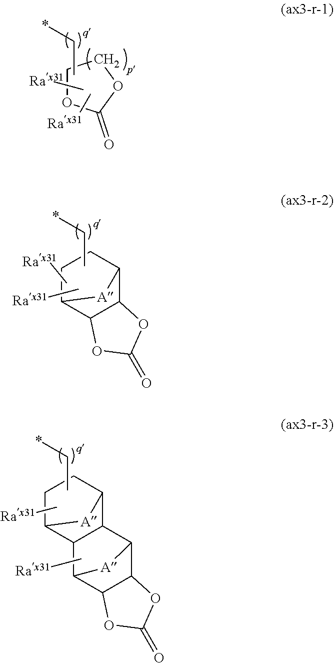

[In the formulae, Ra′^{x31}'s each independently represent a hydrogen atom, an alkyl group, an alkoxy group, a halogen atom, a halogenated alkyl group, a hydroxyl group, —COOR″, —OC(—O)R″, a hydroxyalkyl group, or a cyano group; R″ represents a hydrogen atom, an alkyl group, a lactone-containing cyclic group, a carbonate-containing cyclic group, or an —SO₂— containing cyclic group; A″ represents an oxygen atom, a sulfur atom, or an alkylene group having 1 to 5 carbon atoms, which may have an oxygen atom or a sulfur atom; p′ represents an integer in a range of 0 to 3, and q′ represents 0 or 1. * represents a bonding site.]

In General Formulae (ax3-r-2) and (ax3-r-3), A″ has the same definition as that for A″ in General Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′³¹ include the same groups as those for Ra′²¹ in General Formulae (a2-r-1) to (a2-r-7).

Specific examples of the groups each represented by General Formulae (ax3-r-1) to (ax3-r-3) are shown below.

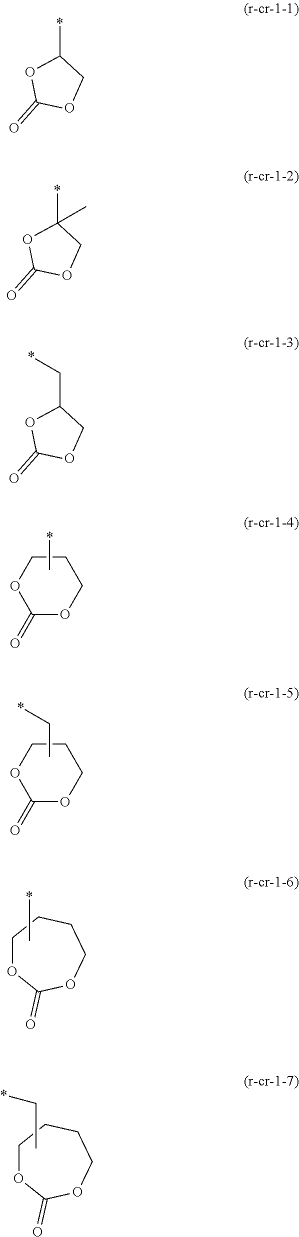

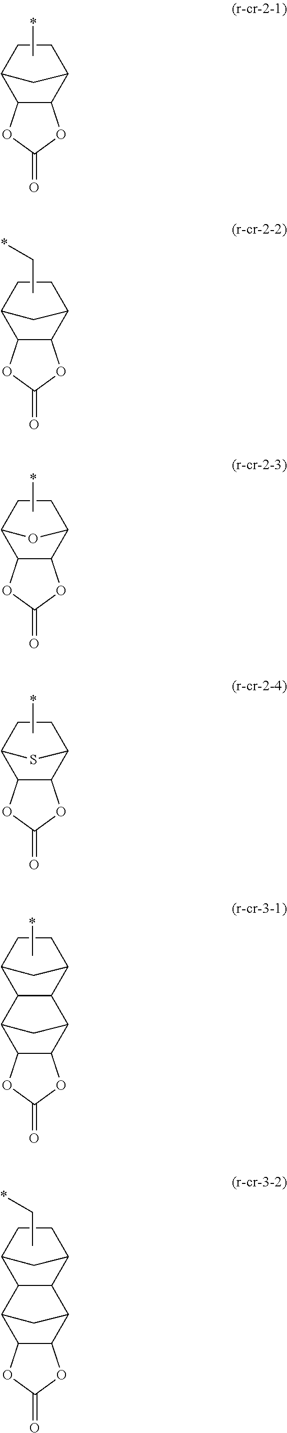

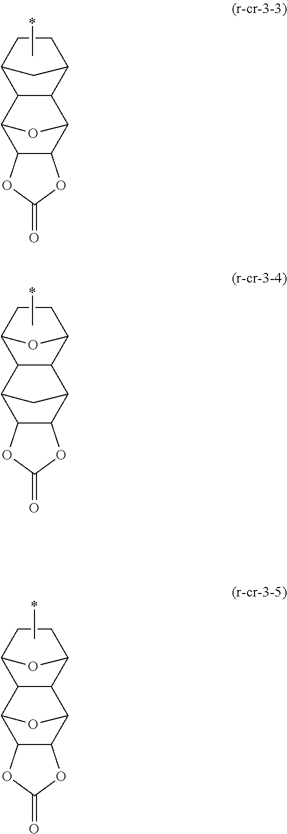

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

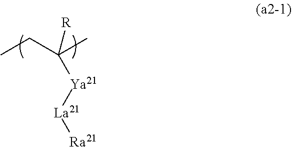

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group, a carbonate-containing cyclic group, or a —SO₂-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a heteroatom.

Divalent Hydrocarbon Group which May have Substituent:

In a case where Ya²¹ represents a divalent hydrocarbon group which may have a substituent, the hydrocarbon group may be an aliphatic hydrocarbon group or an aromatic hydrocarbon group.

### Aliphatic Hydrocarbon Group as Ya²¹

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH)—, —C(CH₃)(CH₂CH₂CH)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. The number of carbon atoms in the polycycloalkane is preferably 7 to 12, and specific examples thereof include adamantane, norbornane, isobornane, tricyclo[5.2.1.0^{2,6}]decane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group as Ya²¹

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom:

In a case where Ya²¹ represents a divalent linking group having a heteroatom, preferred examples of the linking group include —O—, —C(═O)—O—, —O—C(═O)—, —C(═O)—, —O—C(═O)—O—, —C(═O)—NH—, —NH—, —NH—C(═NH)—(H may be substituted with a substituent such as an alkyl group or an acyl group), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²—[in the formulae. Y²¹ and Y²² each independently represent a divalent hydrocarbon group which may have a substituent, O represents an oxygen atom, and m″ represents an integer of 0 to 3].

In a case where the divalent linking group containing a heteroatom is —C(═O)—NH—, —C(═O)—NH—C(═O)—, —NH—, or —NH—C(═NH)—, H may be substituted with a substituent such as an alkyl group, an acyl group, or the like. The substituent (an alkyl group, an acyl group, or the like) has preferably 1 to 10 carbon atoms, more preferably 1 to 8 carbon atoms, and particularly preferably 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]ₘ—Y²²—, —Y²¹—O—C(═O)—Y²²—, or —Y²¹—S(═O)₂—O—Y²²—, Y²¹ and Y²² each independently represent a divalent hydrocarbon group which may have a substituent. Examples of the divalent hydrocarbon group include the same groups as those (the divalent linking group which may have a substituent) described in the section of the divalent linking group as Ya²¹.

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Among the examples, it is preferable that Ya²¹ represents a single bond, an ester bond [—C(═O)—O—], an ether bond (—O—), a linear or branched alkylene group, or a combination thereof.

In Formula (a2-1), Ra²¹ represents a lactone-containing cyclic group, a —SO₂-containing cyclic group, or a carbonate-containing cyclic group.

Suitable examples of the lactone-containing cyclic group, the —SO₂-containing cyclic group, and the carbonate-containing cyclic group as Ra²¹ include groups each represented by General Formulae (a2-r-1) to (a2-r-7), groups each represented by General Formulae (a5-r-1) to (a5-r-4), and groups each represented by General Formulae (ax3-r-1) to (ax3-r-3).

Among the examples, the lactone-containing cyclic group or the —SO₂-containing cyclic group is preferable, a group represented by any of General Formulae (a2-r-1), (a2-r-2), (a2-r-6), and (a5-r-1) is more preferable, and a group represented by any of General Formulae (a2-r-2) and (a5-r-1) is still more preferable. Specifically, a group represented by any of Chemical Formulae (r-lc-1-1) to (r-lc-1-7). (r-lc-2-1) to (r-lc-2-18). (r-lc-6-1), (r-sl-1-1), and (r-sl-1-18) is preferable, a group represented by any of Chemical Formulae (r-lc-2-1) to (r-lc-2-18) and (r-sl-1-1) is more preferable, and a group represented by any of Chemical Formulae (r-lc-2-1). (r-lc-2-12), and (r-sl-1-1) is still more preferable.

The constitutional unit (a2) included in the component (A1) may be used alone or two or more kinds thereof.

In a case where the component (A1) has the constitutional unit (a2), the proportion of the constitutional unit (a2) in the component (A1) is preferably in a range of 5% to 60% by mole, more preferably in a range of 10% to 60% by mole, still more preferably in a range of 20% to 60% by mole, and particularly preferably in a range of 30% to 60% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### In Regard to Constitutional Unit (a3):

The component (A1) may further have a constitutional unit (a3) containing a polar group-containing aliphatic hydrocarbon group (here, those corresponding to the constitutional unit (a1) or the constitutional unit (a2) are excluded) in addition to the constitutional unit (a1). In a case where the component (A1) has the constitutional unit (a3), the hydrophilicity of the component (A) is increased, which contributes to improvement of the resolution. Further, the acid diffusion length can be appropriately adjusted.

Examples of the polar group include a hydroxyl group, a cyano group, a carboxy group, or a hydroxyalkyl group in which some hydrogen atoms in the alkyl group have been substituted with fluorine atoms. Among these, a hydroxyl group is particularly preferable.

Examples of the aliphatic hydrocarbon group include a linear or branched hydrocarbon group (preferably an alkylene group) having 1 to 10 carbon atoms and a cyclic aliphatic hydrocarbon group (cyclic group). The cyclic group may be a monocyclic group or a polycyclic group. For example, the cyclic group can be appropriately selected from the plurality of groups that have been proposed in the resins for resist compositions for ArF excimer lasers and then used.

In a case where the cyclic group is a monocyclic group, the number of carbon atoms is more preferably in a range of 3 to 10. Among the examples, constitutional units derived from acrylic acid ester that includes an aliphatic monocyclic group containing a hydroxyl group, a cyano group, a carboxy group, or a hydroxyalkyl group in which some hydrogen atoms in the alkyl group have been substituted with fluorine atoms are more preferable. Examples of the monocyclic group include groups obtained by removing two or more hydrogen atoms from a monocycloalkane. Specific examples include groups obtained by removing two or more hydrogen atoms from monocycloalkanes such as cyclopentane, cyclohexane, and cyclooctane. Among these monocyclic groups, a group obtained by removing two or more hydrogen atoms from cyclopentane and a group obtained by removing two or more hydrogen atoms from cyclohexane are industrially preferable.

In a case where the cyclic group is a polycyclic group, the number of carbon atoms in the polycyclic group is more preferably in a range of 7 to 30. Among the examples, constitutional units derived from acrylic acid ester that includes an aliphatic polycyclic group containing a hydroxyl group, a cyano group, a carboxy group, or a hydroxyalkyl group in which some hydrogen atoms in the alkyl group have been substituted with fluorine atoms are more preferable. Examples of the polycyclic group include groups in which two or more hydrogen atoms have been removed from bicycloalkane, tricycloalkane, tetracycloalkane, or the like. Specific examples thereof include a group in which two or more hydrogen atoms have been removed from a polycycloalkane such as adamantane, norbornane, isobornane, tricyclo[5.2.1.0^{2,6}]decane, and tetracyclododecane. Among these polycyclic groups, a group in which two or more hydrogen atoms have been removed from adamantane, a group in which two or more hydrogen atoms have been removed from norbornane, or a group in which two or more hydrogen atoms have been removed from tetracyclododecane is industrially preferable.

The constitutional unit (a3) is not particularly limited as long as the constitutional unit contains a polar group-containing aliphatic hydrocarbon group, and an optional constitutional unit can be used.

As the constitutional unit (a3), a constitutional unit derived from acrylic acid ester in which the hydrogen atom bonded to the carbon atom at the (1-position may be substituted with a substituent, which is a constitutional unit containing a polar group-containing aliphatic hydrocarbon group is preferable.

In a case where the hydrocarbon group in the polar group-containing aliphatic hydrocarbon group is a linear or branched hydrocarbon group having 1 to 10 carbon atoms, a constitutional unit derived from hydroxyethyl ester of acrylic acid is preferable as the constitutional unit (a3).

Further, in a case where the hydrocarbon group in the polar group-containing aliphatic hydrocarbon group is a polycyclic group, a constitutional unit represented by Formula (a3-1), a constitutional unit represented by Formula (a3-2), or a constitutional unit represented by Formula (a3-3) is preferable as the constitutional unit (a3). Further, in a case where the hydrocarbon group in the polar group-containing aliphatic hydrocarbon group is a monocyclic group, a constitutional unit represented by Formula (a3-4) is preferable as the constitutional unit (a3).

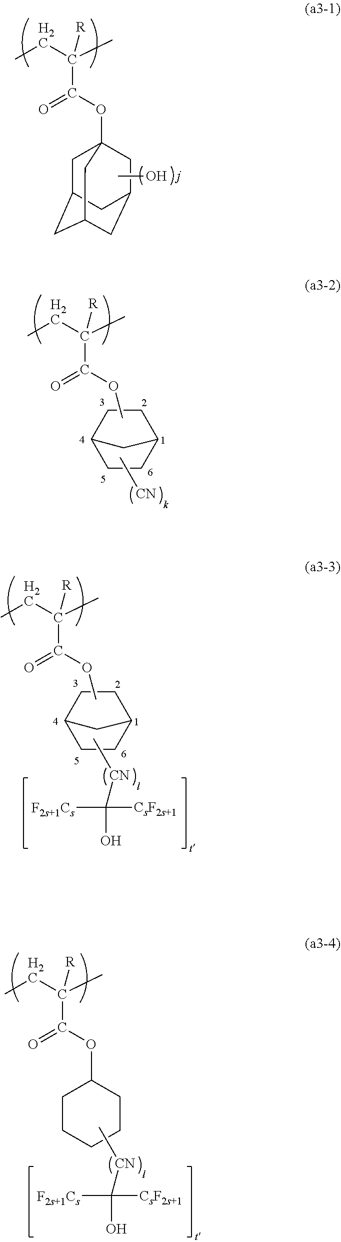

[In the formulae, R has the same definition as described above, j represents an integer of 1 to 3, k represents an integer of 1 to 3, t′ represents an integer of 1 to 3, 1 represents an integer of 0 to 5, and s represents an integer of 1 to 3.]

In Formula (a3-1) j represents preferably 1 or 2 and more preferably 1. In a case where j represents 2, it is preferable that the hydroxyl groups are bonded to the 3- and 5-positions of the adamantyl group. In a case where j represents 1, it is preferable that the hydroxyl group is bonded to the 3-position of the adamantyl group.

It is preferable that j represents 1, and it is particularly preferable that the hydroxyl group is bonded to the 3-position of the adamantyl group.

In Formula (a3-2), it is preferable that k represents 1. It is preferable that the cyano group is bonded to the 5- or 6-position of the norbornyl group.

In Formula (a3-3), it is preferable that t′ represents 1. It is preferable that l represents 1. It is preferable that s represents 1. Further, it is preferable that a 2-norbornyl group or 3-norbornyl group is bonded to the terminal of the carboxy group of the acrylic acid. It is preferable that the fluorinated alkyl alcohol is bonded to the 5- or 6-position of the norbornyl group.

In Formula (a3-4), it is preferable that t′ represents 1 or 2. It is preferable that 1 represents 0 or 1. It is preferable that s represents 1. It is preferable that the fluorinated alkyl alcohol is bonded to the 3- or 5-position of the cyclohexyl group.

The constitutional unit (a3) included in the component (A1) may be used alone or two or more kinds thereof.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 20% by mole with respect to the total amount (100% by mole) of all constitutional units constituting the component (A1).

In a case where the proportion of the constitutional unit (a3) is set to be greater than or equal to the lower limits of the above-described preferable ranges, the effect to be obtained by allowing the component (A1) to have the constitutional unit (a3) is sufficiently obtained by the above-described effects. Further, in a case where the proportion thereof is set to be less than or equal to the upper limits of the above-described preferable ranges, the constitutional unit (a3) and other constitutional units can be balanced, and the lithography characteristics are improved.

### In Regard to Constitutional Unit (a4):

The component (A1) may further have a constitutional unit (a4) containing an acid non-dissociable aliphatic cyclic group in addition to the constitutional unit (a1).

In a case where the component (A1) has the constitutional unit (a4), the dry etching resistance of a resist pattern to be formed is improved. Further, the hydrophobicity of the component (A) is increased. The improvement of the hydrophobicity contributes to improvement of the resolution, the resist pattern shape, and the like particularly in a case of the solvent developing process.

The term “acid non-dissociable cyclic group” in the constitutional unit (a4) denotes a cyclic group remaining in the constitutional unit without being dissociated by the action of an acid in a case of generation of an acid in the resist composition upon light exposure (for example, an acid is generated from the component (B) or a constitutional unit that generates an acid upon light exposure).

As the constitutional unit (a4), for example, a constitutional unit derived from acrylic acid ester containing an acid non-dissociable aliphatic cyclic group or the like is preferable. As the cyclic group, a plurality of cyclic groups which have been known in the related art as those used for resin components of resist compositions for an ArF excimer laser, a KrF excimer laser (preferably an ArF excimer laser), and the like can be used.

It is particularly preferable that the cyclic group is at least one selected from a tricyclodecyl group, an adamantyl group, a tetracyclododecyl group, an isobornyl group, and a norbornyl group from the viewpoint of the industrial availability or the like. These polycyclic groups may have a linear or branched alkyl group having 1 to 5 carbon atoms as a substituent.

Specific examples of the constitutional unit (a4) include constitutional units each represented by General Formulae (a4-1) to (a4-7).

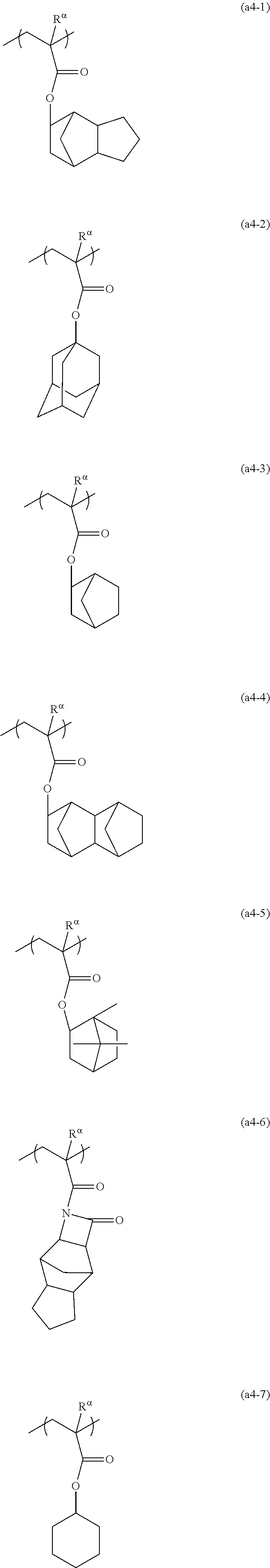

[In the formulae, R^{α} has the same definition as described above.]

The constitutional unit (a4) included in the component (A1) may be used alone or two or more kinds thereof.

In a case where the component (A1) contains the constitutional unit (a4), the proportion of the constitutional unit (a4) is preferably in a range of 1% to 40% by mole and more preferably in a range of 5% to 20% by mole with respect to the total amount (100% by mole) of all constitutional units constituting the component (A1).

In a case where the proportion of the constitutional unit (a4) is set to be greater than or equal to the lower limits of the above-described preferable ranges, the effect to be obtained by allowing the component (A1) to have the constitutional unit (a4) is sufficiently obtained by the above-described effects. Further, in a case where the proportion thereof is set to be less than or equal to the upper limits of the above-described preferable ranges, the constitutional unit (a4) and other constitutional units are likely to be balanced.

### In Regard to Constitutional Unit (St):

The constitutional unit (st) is a constitutional unit derived from styrene or a styrene derivative thereof. The phrase “constitutional unit derived from styrene” means a constitutional unit that is formed by the cleavage of an ethylenic double bond of styrene. The phrase “constitutional unit derived from a styrene derivative” means a constitutional unit formed by the cleavage of an ethylenic double bond of a styrene derivative.

The term “styrene derivative” denotes a compound in which at least some hydrogen atoms of styrene are substituted with a substituent. Examples of the styrene derivative include those in which the hydrogen atom at the α-position of styrene is substituted with a substituent, those in which one or more hydrogen atoms in the benzene ring of styrene are substituted with a substituent, and those in which the hydrogen atom at the α-position of styrene and one or more hydrogen atoms of the benzene ring are substituted with a substituent.

Examples of the substituent that substitutes the hydrogen atom at the α-position of styrene include an alkyl group having 1 to 5 carbon atoms and a halogenated alkyl group having 1 to 5 carbon atoms.

As the alkyl group having 1 to 5 carbon atoms, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

As the substituent that substitutes the hydrogen atom at the α-position of styrene, an alkyl group having 1 to 5 carbon atoms or a fluorinated alkyl group having 1 to 5 carbon atoms is preferable, an alkyl group having 1 to 3 carbon atoms or a fluorinated alkyl group having 1 to 3 carbon atoms is more preferable, and a methyl group is still more preferable from the viewpoint of industrial availability.

Examples of substituents that substitute the hydrogen atom of the benzene ring of styrene include an alkyl group, an alkoxy group, a halogen atom, and a halogenated alkyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

As the substituent that substitutes the hydrogen atom of the benzene ring of styrene, an alkyl group having 1 to 5 carbon atoms is preferable, a methyl group or an ethyl group is more preferable, and a methyl group is still more preferable.

As the constitutional unit (st), a constitutional unit derived from styrene or a constitutional unit derived from a styrene derivative in which the hydrogen atom at the α-position of styrene is substituted with an alkyl group having 1 to 5 carbon atoms or a halogenated alkyl group having 1 to 5 carbon atoms is preferable, a constitutional unit derived from styrene or a constitutional unit derived from a styrene derivative in which the hydrogen atom at the α-position of styrene is substituted with a methyl group is more preferable, and a constitutional unit derived from styrene is still more preferable.

The constitutional unit (st) included in the component (A1) may be used alone or two or more kinds thereof.

In a case where the component (A1) has the constitutional unit (st), the proportion of the constitutional unit (st) is preferably in a range of 1% to 30% by mole and more preferably in a range of 3% to 20% by mole with respect to the total amount (100% by mole) of all constitutional units constituting the component (A1).

The component (A1) contained in the resist composition may be used alone or in combination of two or more kinds thereof.

In the resist composition according to the present embodiment, examples of the component (A1) include a polymer compound having a repeating structure of the constitutional unit (a1), and preferred examples thereof include a polymer compound having a repeating structure of the constitutional unit (a1) and the constitutional unit (a2).

Among these, suitable examples of the component (A1) include a polymer compound consisting of a repeating structure of a constitutional unit (a1) and a constitutional unit (a2); and a polymer compound consisting of a repeating structure of a constitutional unit (a1), a constitutional unit (a2), and a constitutional unit (a3).

In the polymer compound having a repeating structure of the constitutional unit (a1) and the constitutional unit (a2), the proportion of the constitutional unit (a1) is preferably in a range of 10% to 90% by mole, more preferably in a range of 20% to 80% by mole, still more preferably in a range of 30% to 70% by mole, and particularly preferably in a range of 40% to 70% by mole with respect to the total amount (100% by mole) of all constitutional units constituting the polymer compound.

In addition, the proportion of the constitutional unit (a2) in each of the polymer compounds described above is preferably in a range of 10% to 90% by mole, more preferably in a range of 20% to 80% by mole, still more preferably in a range of 30% to 70% by mole, and particularly preferably in a range of 30% to 60% by mole, with respect to the total amount (100% by mole) of all constitutional units constituting the polymer compound.

In the polymer compound having a repeating structure of the constitutional unit (a1), the constitutional unit (a2), and the constitutional unit (a3), the proportion of the constitutional unit (a1) is preferably in a range of 20% to 80% by mole, more preferably in a range of 30% to 70% by mole, still more preferably in a range of 40% to 60% by mole, and particularly preferably in a range of 45% to 55% by mole with respect to the total amount (100% by mole) of all constitutional units constituting the polymer compound.

In addition, the proportion of the constitutional unit (a2) in each of the polymer compounds described above is preferably in a range of 10% to 70% by mole, more preferably in a range of 20% to 60% by mole, still more preferably in a range of 30% to 50% by mole, and particularly preferably in a range of 35% to 45% by mole, with respect to the total amount (100% by mole) of all constitutional units constituting the polymer compound.

In addition, the proportion of the constitutional unit (a3) in the polymer compound described above is preferably in a range of 1% to 30% by mole, more preferably in a range of 5% to 25% by mole, still more preferably in a range of 5% to 20% by mole, and particularly preferably in a range of 5% to 15% by mole, with respect to the total amount (100% by mole) of all constitutional units constituting the polymer compound.

The molar ratio of the constitutional unit (a1) to the constitutional unit (a2) in the polymer compound (the constitutional unit (a1):the constitutional unit (a2)) is preferably in a range of 2:8 to 8:2, more preferably in a range of 3:7 to 7:3, and still more preferably in a range of 4:6 to 6:4.

Such a component (A1) can be produced by dissolving a monomer, from which each constitutional unit is derived, in a polymerization solvent and adding a radical polymerization initiator such as azobisisobutyronitrile (AIBN) or dimethyl azobisisobutyrate (for example, V-601) to the solution so that the polymerization is carried out.

Alternatively, such a component (A1) can be produced by dissolving a monomer from which the constitutional unit (a1) is derived and a monomer from which constitutional units (for example, the constitutional unit (a2)) other than the constitutional unit (a1) are derived as necessary in a polymerization solvent, adding the above-described radical polymerization initiator to the solution, and performing polymerization. Further, a —C(CF₃)₂—OH group may be introduced to the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, and still more preferably in a range of 3000 to 20000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and improved roughness is likely to be formed.

In the resist composition of the present embodiment, the content of the component (A) may be adjusted according to the thickness of the resist film intended to be formed.

### <Component (D0)>

The component (D0) is a compound represented by General Formula (d0).

In the resist composition according to the present embodiment, the component (D0) is a base component which acts as a quencher (acid diffusion control agent) that traps the acid generated upon light exposure. The component (D0) is a photodecomposable base that is decomposed upon light exposure and loses acid diffusion controllability, and does not act as a quencher in the exposed portion of the resist film because the acid diffusion controllability (basicity) is lost by the decomposition, but acts as a quencher in the unexposed portion of the resist film.

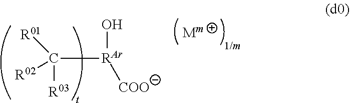

[In the formula, R^{Ar} represents a (t+2)-valent aromatic hydrocarbon group which may have a substituent. t represents an integer of 1 or greater. R⁰¹ and R⁰² each independently represent a chain-like hydrocarbon group, R⁰³ represents a chain-like hydrocarbon group or a hydrogen atom, or two or more of R⁰¹, R⁰², and R⁰³ are bonded to each other to form a ring structure. Here, in a case where two of R⁰¹, R⁰², and R⁰¹ are bonded to each other to form a ring structure, the ring structure is an alicyclic ring, and the remaining one represents a chain-like hydrocarbon group or a hydrogen atom. The chain-like hydrocarbon group may have a substituent. The ring structure may have a substituent. m represents an integer of 1 or greater, and M^{m+} represents an m-valent organic cation.]

In Formula (d0), R^{Ar} represents a (t+2)-valent aromatic hydrocarbon group which may have a substituent.

Examples of the aromatic hydrocarbon group as R^{Ar} include a group in which (t+2) hydrogen atoms have been removed from an aromatic ring which may have a substituent.

The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

In addition, examples of the aromatic hydrocarbon group in R^{Ar} also include a group in which (t+2) hydrogen atoms have been removed from an aromatic compound (such as biphenyl or fluorene) having an aromatic ring which may have a substituent.

Among the examples, R^{Ar} represents preferably a group in which (t+2) hydrogen atoms have been removed from benzene, naphthalene, anthracene, or biphenyl, more preferably a group in which (t+2) hydrogen atoms have been removed from benzene or naphthalene, and still more preferably a group in which (t+2) hydrogen atoms have been removed from benzene.

The aromatic hydrocarbon group as R^{Ar} may or may not have a substituent. Examples of the substituent include a linear alkyl group, an alkoxy group, a halogen atom, and a halogenated alkyl group.

Examples of the linear alkyl group, the alkoxy group, the halogen atom, and the halogenated alkyl group as the substituents include the same substituents (the alkyl group, the alkoxy group, the halogen atom, and the halogenated alkyl group) of the cyclic aliphatic hydrocarbon group represented by Ya²¹ as described above.

As the linear alkyl group as the substituent, a linear alkyl group having 1 to 5 carbon atoms is preferable, and a methyl group, an ethyl group, a propyl group, an n-butyl group, or a tert-butyl group is most preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include groups in which some or all hydrogen atoms in the above-described alkyl groups have been substituted with the above-described halogen atoms.

It is preferable that the aromatic hydrocarbon group as R^{Ar} has no substituent.

In Formula (d0), t represents an integer of 1 or greater, and is appropriately determined depending on the aromatic hydrocarbon group as R^{Ar}. For example, t may represent an integer of 1 to 5, preferably an integer of 1 to 4, more preferably 1 or 2, and particularly preferably 1.

In Formula (d0), R⁰¹ and R⁰² each independently represent a chain-like hydrocarbon group, R⁰³ represents a chain-like hydrocarbon group or a hydrogen atom, or two or more of R⁰¹, R⁰², and R⁰³ are bonded to each other to form a ring structure. Here, in a case where two of R⁰¹, R⁰², and R⁰³ are bonded to each other to form a ring structure, the ring structure is an alicyclic ring, and the remaining one represents a chain-like hydrocarbon group or a hydrogen atom. The chain-like hydrocarbon group may have a substituent. The ring structure may have a substituent.

Each of the chain-like hydrocarbon groups as R⁰¹, R⁰², and R⁰³ may be a saturated aliphatic hydrocarbon group or an unsaturated aliphatic hydrocarbon group, but is preferably a saturated aliphatic hydrocarbon group, and examples thereof include a linear or branched alkyl group.

In addition, each of the chain-like hydrocarbon groups as R⁰¹, R⁰², and R⁰³ may be linear or branched, and is preferably linear.

Examples of the saturated aliphatic hydrocarbon group as R01, R02, and R03 include a linear or branched alkyl group, and specific examples thereof include a linear alkyl group such as a methyl group, an ethyl group, a propyl group, an n-butyl group, or an n-pentyl group; and a branched alkyl group such as an isopropyl group, an isobutyl group, a tert-butyl group, an isopentyl group, or a neopentyl group. Among these, a methyl group, an ethyl group, a propyl group, an isopropyl group, an n-butyl group, an isobutyl group, or a tert-butyl group is preferable, a methyl group or an ethyl group is more preferable, and a methyl group is still more preferable.

Examples of the unsaturated aliphatic hydrocarbon group as R⁰¹, R⁰², and R⁰³ include a linear or branched alkenyl group, and examples thereof include a linear alkenyl group such as a vinyl group, a propenyl group (allyl group), or a 2-butenyl group; and a branched alkenyl group such as a 1-methylvinyl group, a 2-methylvinyl group, a 1-methylpropenyl group, or a 2-methylpropenyl group.

The chain-like hydrocarbon groups as R⁰¹, R⁰², and R⁰³ may have a substituent.

Examples of the substituent that such a chain-like hydrocarbon group may have include a halogen atom and a halogenated alkyl group. Examples of the halogen atom as the substituent include a fluorine atom, a chlorine atom, a bromine atom, and an iodine atom. Examples of the halogenated alkyl group as the substituent include groups in which some or all hydrogen atoms in the above-described alkyl groups have been substituted with the above-described halogen atoms.

The ring structure formed by two or more of R⁰¹, R⁰², and R⁰³ being bonded to each other may be an aromatic ring or an alicyclic ring. Here, in a case where two of R⁰¹, R⁰², and R⁰³ are bonded to each other to form a ring structure, the ring structure is an alicyclic ring.

The aromatic ring formed by two or more of R⁰¹, R⁰², and R⁰³ being bonded to each other has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom.

The alicyclic ring formed by two or more of R⁰¹, R⁰², and R⁰³ being bonded to each other has preferably 3 to 20 carbon atoms and more preferably 3 to 12 carbon atoms. The alicyclic ring in this case may have a monocyclic structure or a polycyclic structure. As the monocyclic structure, a monocycloalkane is preferable. The monocycloalkane has preferably 3 to 6 carbon atoms, and specific examples thereof include cyclopentane and cyclohexane. As the polycyclic structure, a polycycloalkane is preferable. The polycycloalkane is preferably a polycycloalkane having 7 to 12 carbon atoms, and specific examples thereof include adamantane, norbornane, isobornane, tricyclo[5.2.1.0^{2,6}]decane, and tetracyclododecane.

The ring structure formed by two or more of R⁰¹, R⁰², and R⁰³ being bonded to each other may have a substituent. Examples of the substituent that the ring structure may have include an alkyl group, an alkoxy group, a halogen atom, and a halogenated alkyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include groups in which some or all hydrogen atoms in the above-described alkyl groups have been substituted with the above-described halogen atoms.

Among the examples, as the substituent that the ring structure may have, a halogen atom is preferable, and a fluorine atom is more preferable.

Hereinafter, suitable specific examples of the anion moiety of the compound (component (D0)) represented by General Formula (d0) will be shown in a case where R⁰¹ and R⁰² each independently represent a chain-like hydrocarbon group and R⁰³; represents a chain-like hydrocarbon group or a hydrogen atom.

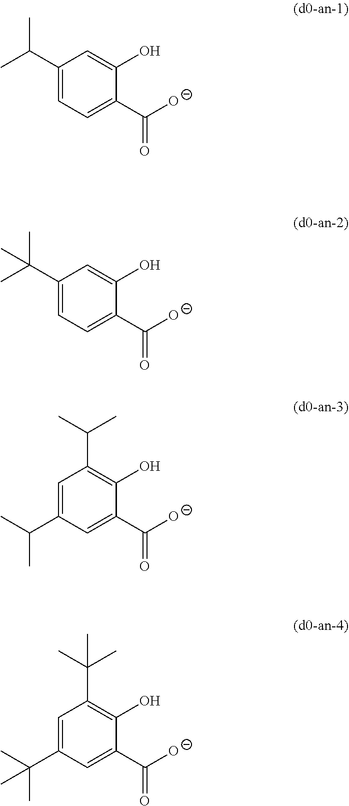

Hereinafter, suitable specific examples of the anion moiety of the compound (component (D0)) represented by General Formula (d0) will be shown in a case where two or more of R⁰¹, R⁰², and R⁰³ are bonded to each other to form an aromatic ring as a ring structure.

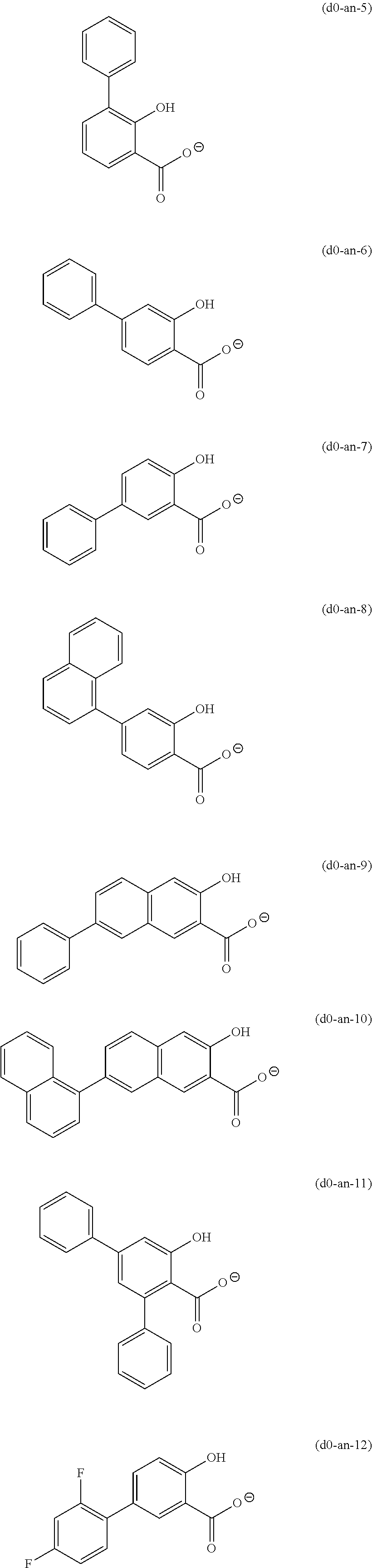

Hereinafter, suitable specific examples of the anion moiety of the compound (component (D0)) represented by General Formula (d0) will be shown in a case where two of R⁰¹, R⁰², and R⁰³ are bonded to each other to form an alicyclic ring as a ring structure and the remaining one represents a chain-like hydrocarbon group or a hydrogen atom.

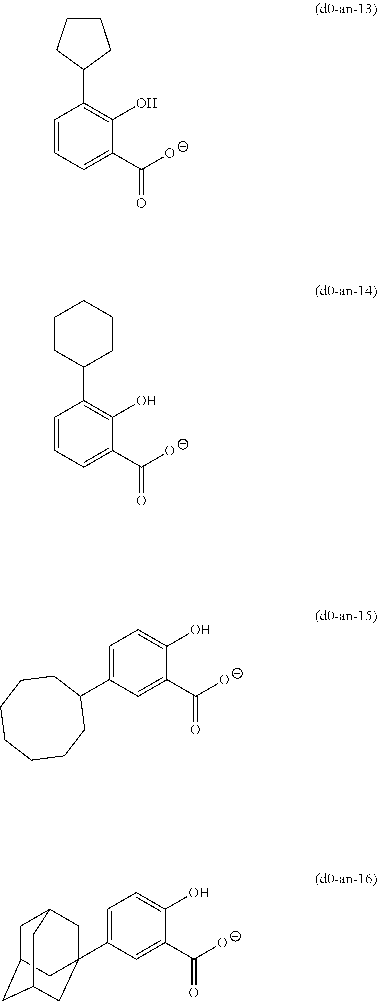

In Formula (d0), m represents an integer of 1 or greater, and M^{m+} represents an m-valent organic cation, preferably an m-valent onium cation, and more preferably a sulfonium cation or an iodonium cation.

Preferred examples of the cation moiety ((M^{m+})_{1/m}) include organic cations each represented by General Formulae (ca-1) to (ca-3).

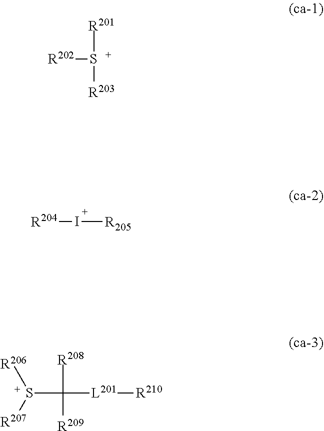

[In the formula, R²⁰¹ to R²⁰⁷ each independently represent an aryl group which may have a substituent, an alkyl group which may have a substituent, or an alkenyl group which may have a substituent. R²⁰¹ to R²⁰³, and R²⁰⁶ and R²⁰⁷ may be bonded to each other to form a ring with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms. Among these, a phenyl group or a naphthyl group is preferable.

As the alkyl group as R²⁰¹ to R²⁰⁷, a chain-like or cyclic alkyl group having 1 to 30 carbon atoms is preferable.

It is preferable that the alkenyl group as R²⁰¹ to R²⁰⁷ has 2 to 10 carbon atoms.

Examples of the substituent that R²⁰¹ to R²⁰⁷ and R²¹⁰ may have include an alkyl group, a halogen atom, a halogenated alkyl group, a carbonyl group, a cyano group, an amino group, an aryl group, and a group represented by any of General Formulae (ca-r-1) to (ca-r-8).

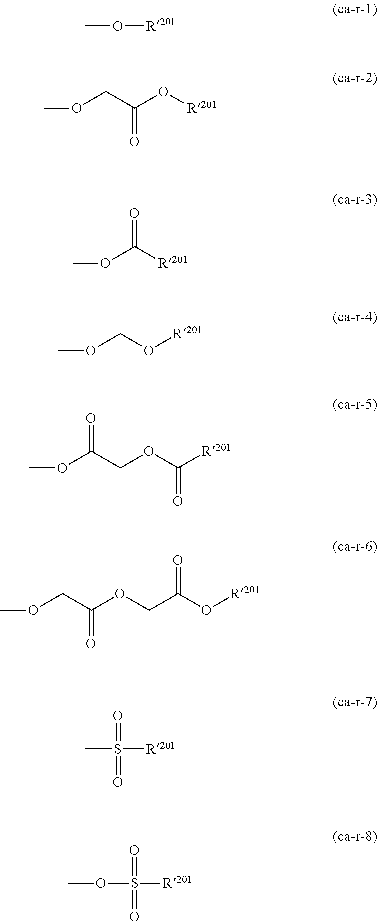

[In the formulae, R′²⁰¹'s each independently represent a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings are substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and heterocyclic groups each represented by Chemical Formulae (r-hr-1) to (r-hr-16).

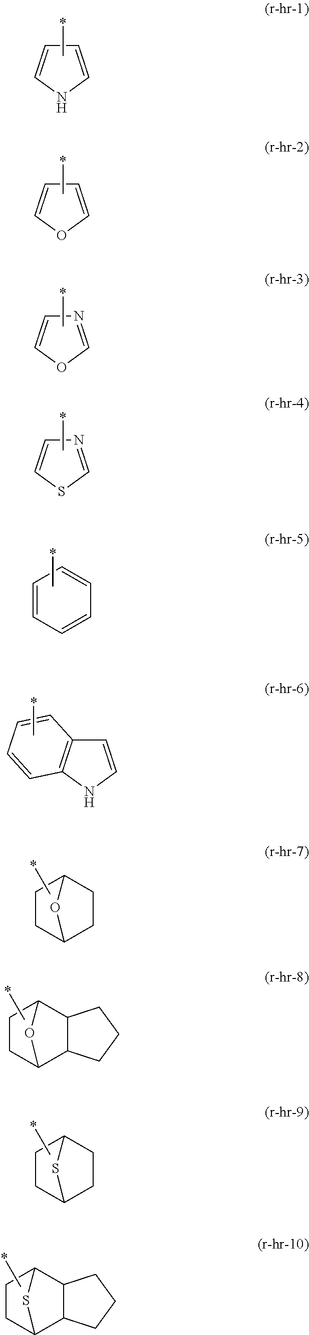

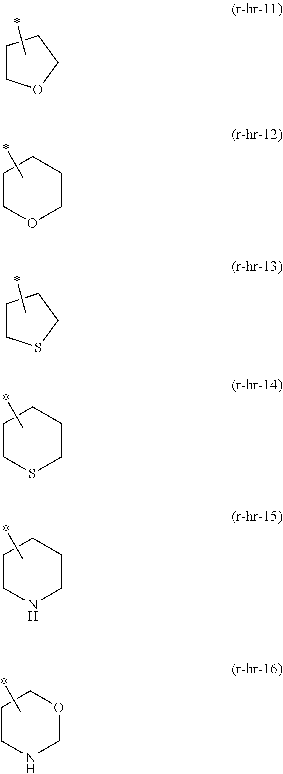

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH— or —N(R_{N})—(R_{N} represents an alkyl group having 1 to 5 carbon atoms). As a ring to be formed, one ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ s and R²⁰⁹ may be bonded to each other to form a ring.

R²¹⁰ represents an aryl group which may have a substituent, an alkyl group which may have a substituent, an alkenyl group which may have a substituent, or a SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ has 2 to 10 carbon atoms.

As the SO₂-containing cyclic group as R²¹⁰ which may have a substituent, “—SO₂-containing polycyclic group” is preferable, and a group represented by General Formula (a5-r-1) is more preferable.

Specific examples of suitable cations represented by Formula (ca-1) include cations each represented by Chemical Formulae (ca-1-1) to (ca-1-70).

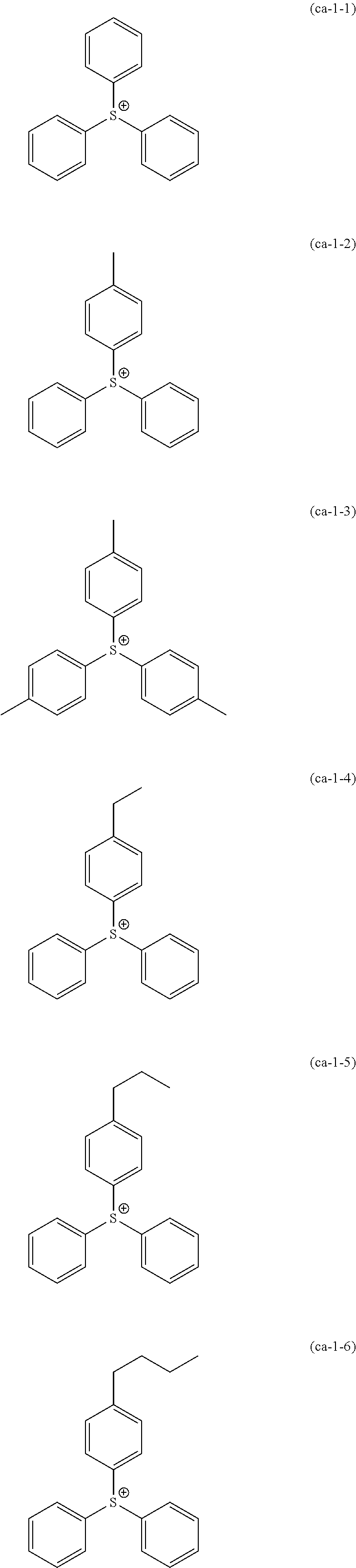

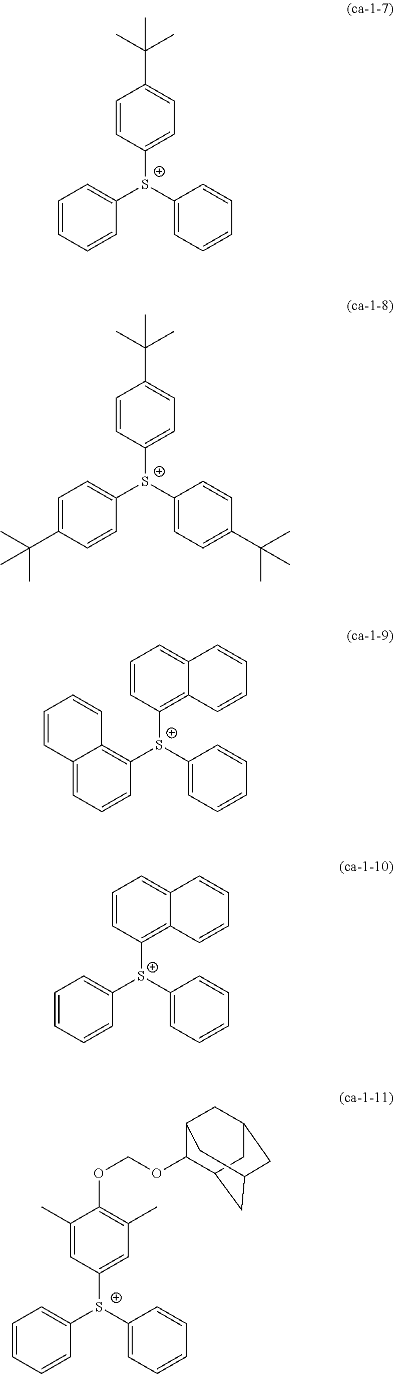 [SMILES: CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1 ; Cc1cc([S+](c2ccccc2)c2ccccc2)cc(C)c1OCOC1C2CC3CC(C2)CC1C3]

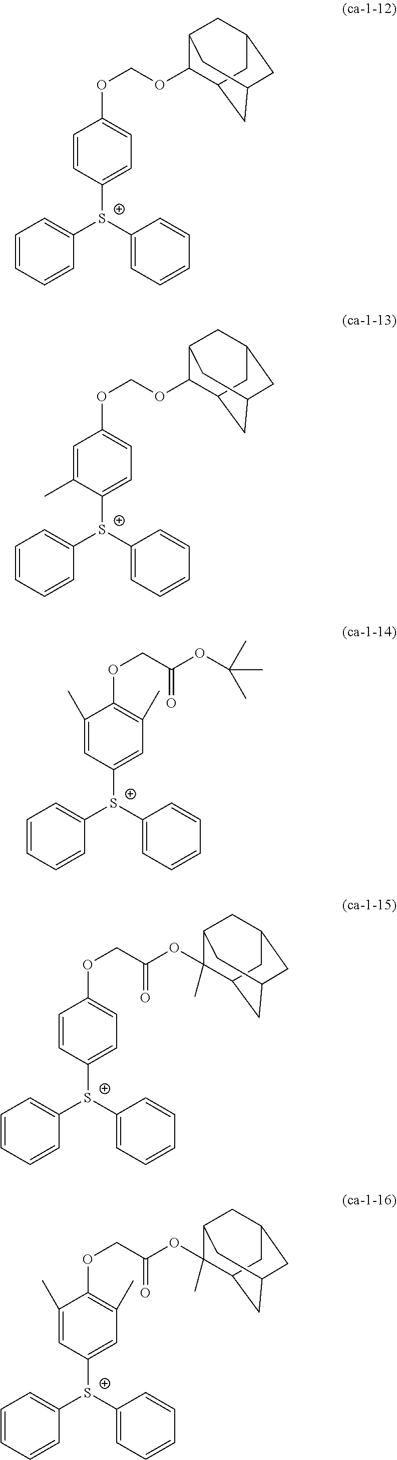 [SMILES: c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C ; CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3]

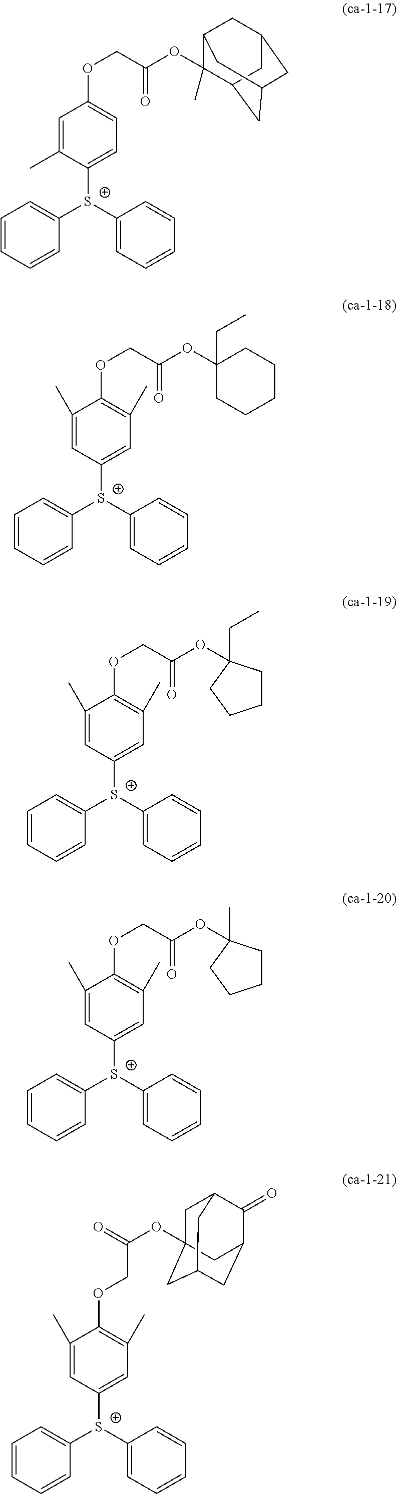 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1 ; Cc1cc([S+](c2ccccc2)c2ccccc2)cc(C)c1OCC(=O)OC12CC3CC(C1)C(=O)C(C3)C2]

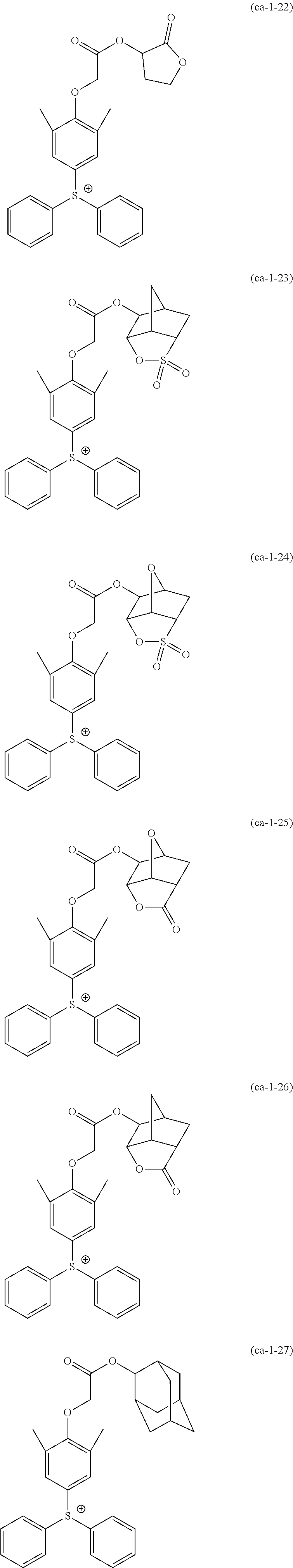 [SMILES: Cc1cc([S+](c2ccccc2)c2ccccc2)cc(C)c1OCC(=O)OC1CCOC1=O ; Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2 ; Cc1cc([S+](c2ccccc2)c2ccccc2)cc(C)c1OCC(=O)OC1C2CC3C(=O)OC1C3C2 ; Cc1cc([S+](c2ccccc2)c2ccccc2)cc(C)c1OCC(=O)OC1C2CC3CC(C2)CC1C3]

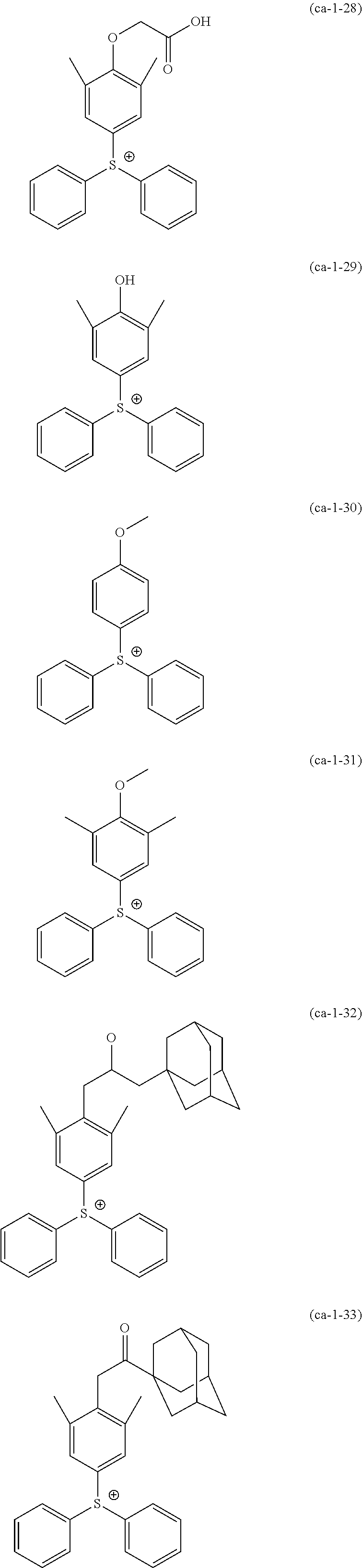 [SMILES: Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1 ; COc1c(C)cc([S+](c2ccccc2)c2ccccc2)cc1C ; Cc1cc([S+](c2ccccc2)c2ccccc2)cc(C)c1CC(O)CC12CC3CC(CC(C3)C1)C2 ; Cc1cc([S+](c2ccccc2)c2ccccc2)cc(C)c1CC(=O)C12CC3CC(CC(C3)C1)C2]

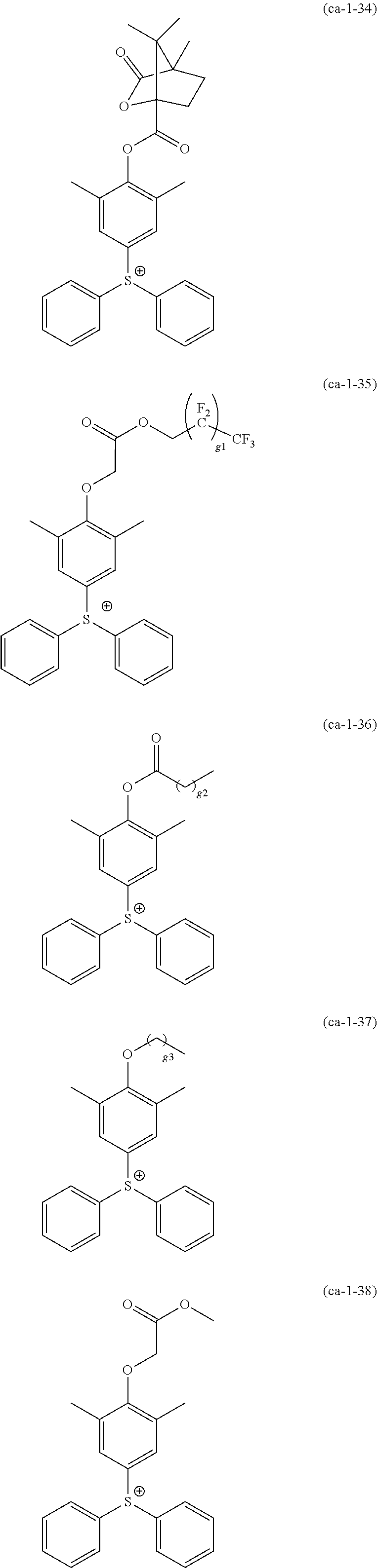

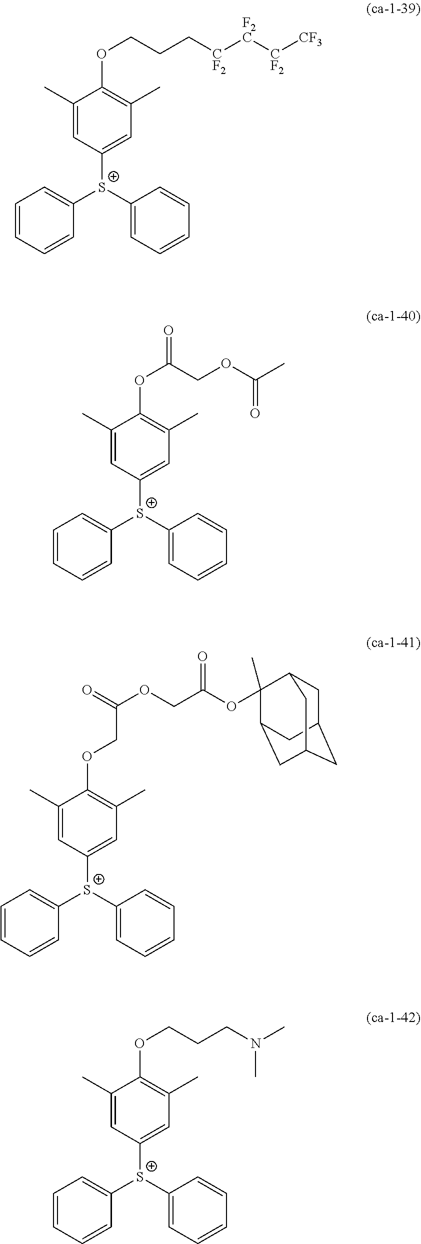 [SMILES: Cc1cc([S+](c2ccccc2)c2ccccc2)cc(C)c1OCCCC(F)(F)C(F)(F)C(F)(F)C(F)(F)F ; CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C]

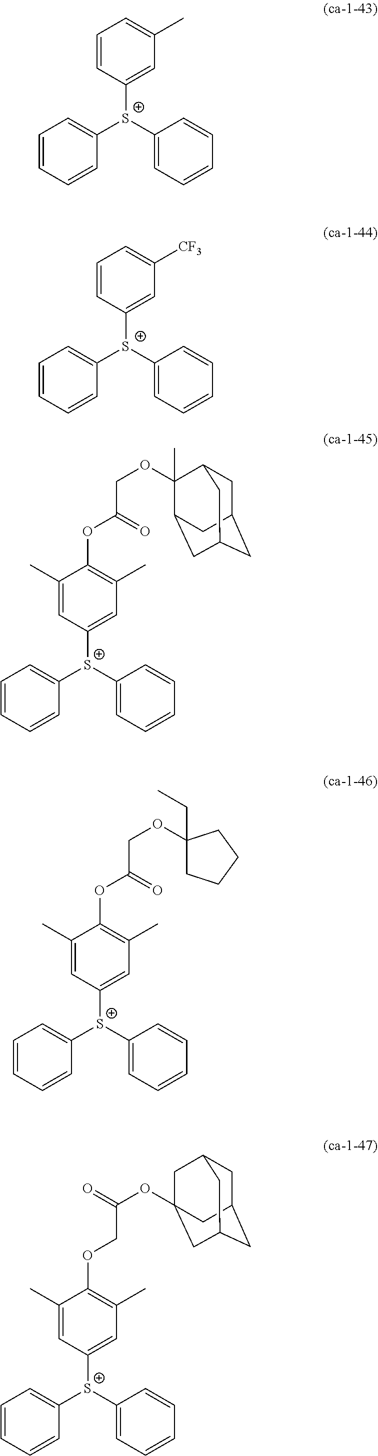 [SMILES: Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3 ; CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

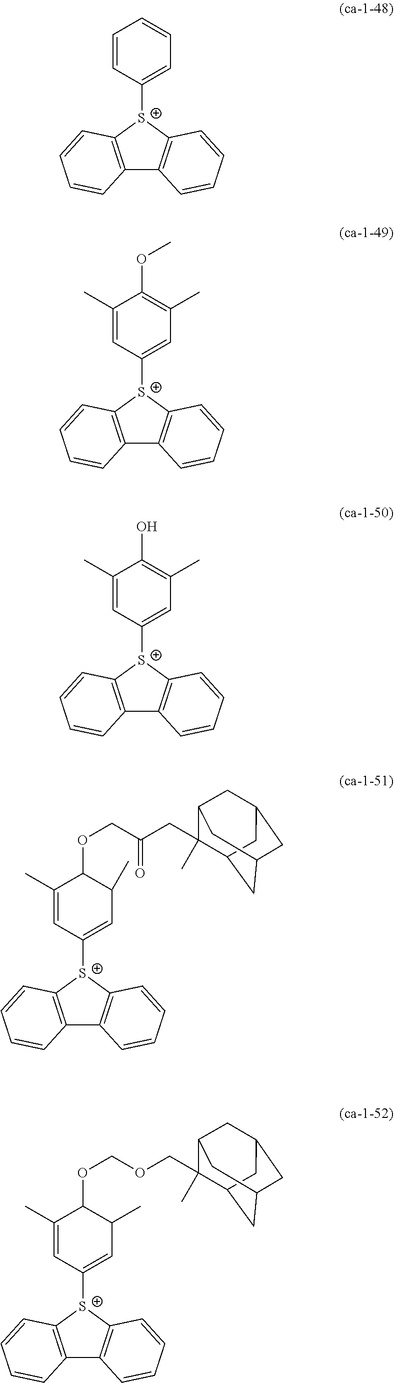 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)CC1(C)C2CC3CC(C2)CC1C3 ; CC1=CC([s+]2c3ccccc3c3ccccc32)=CC(C)C1OCOCC1(C)C2CC3CC(C2)CC1C3]

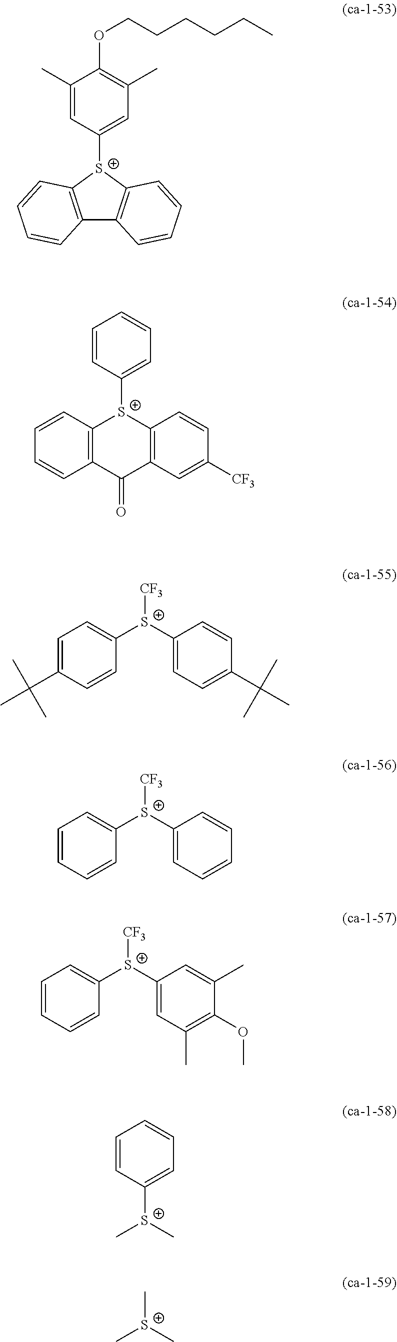

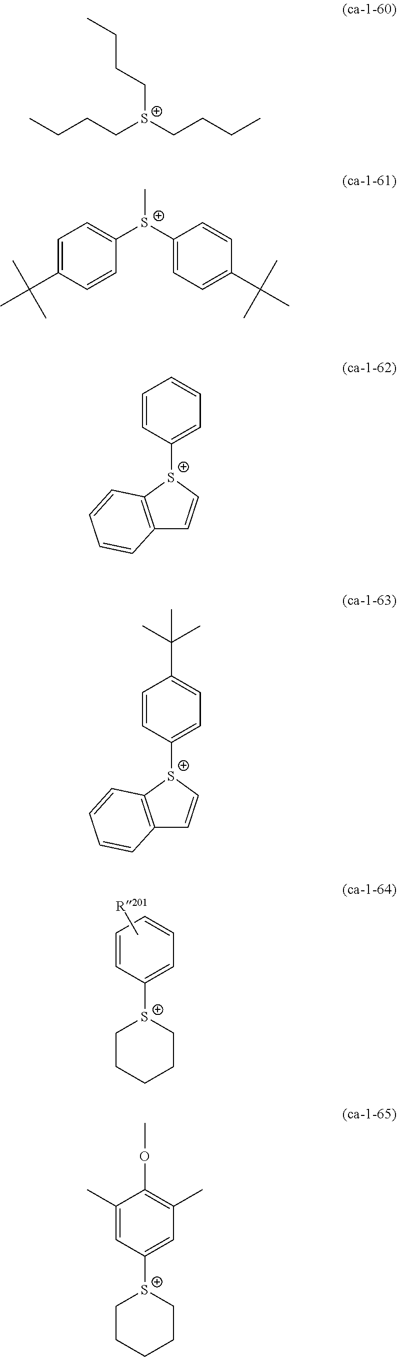

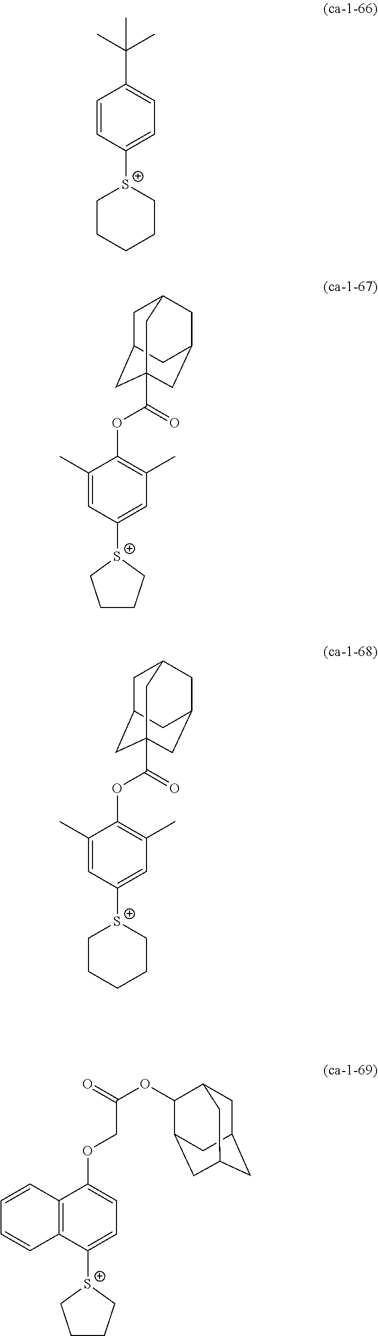 [SMILES: CC(C)(C)c1ccc([S+]2CCCCC2)cc1 ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

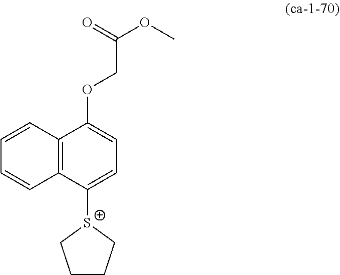 [SMILES: COC(=O)COc1ccc([S+]2CCCC2)c2ccccc12]

[In the formulae, R″²⁰¹ represents a hydrogen atom or a substituent, and examples of the substituent include the same groups as those for the substituents that R²⁰¹ to R²⁰⁷ and R²¹⁰ may have.]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

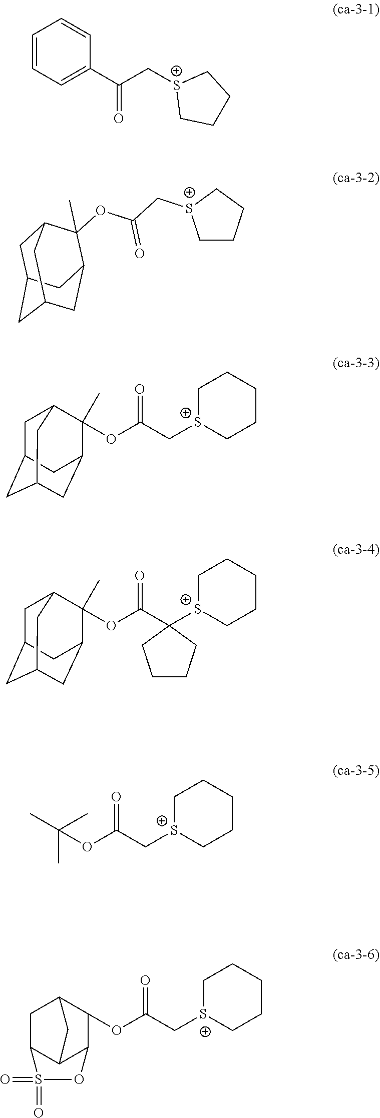

Among the examples, the cation moiety (M^{m+})_{1/m} is preferably a cation represented by General Formula (ca-1) and more preferably a cation represented by any of General Formulae (ca-1-1) to (ca-1-70).

In the resist composition according to the present embodiment, the component (D0) is preferably a component having an anion moiety with high hydrophobicity.

For example, a distance (Ra) between interactions of the Hansen solubility parameter of the compound in which the anion moiety of the component (D0) is protonated (compound in which a proton is bonded to the anion moiety of the component (D0)) and the Hansen solubility parameter of water is preferably 32 or greater.

The distance (Ra) between the interactions of the Hansen solubility parameter of the component (D0) and the Hansen solubility parameter of water may be 32.5 or greater, or 33 or greater, or 33 or greater and 37 or less.

In a case where the distance (Ra) between the interactions is in the above-described preferable ranges, the occurrence of defects is more likely to be suppressed.

The Hansen solubility parameter can be calculated from predetermined parameters based, for example, on solubility parameters and aggregation properties as described by Charles Hansen in Charles M. Hansen, “Hansen Solubility Parameters: A User's Handbook”, CRC Press (2007) and “The CRC Handbook and Solubility Parameters and Cohesion Parameters,” (1999) edited by Allan F. M. Barton (1999).

The Hansen solubility parameter is theoretically calculated as a numerical constant and is a useful tool for predicting the ability of a solvent material to dissolve a particular solute.

The Hansen solubility parameters can be a measure of the overall strength and selectivity of a material by combining the following three experimentally and theoretically derived Hansen solubility parameters (that is, δD, δP, and δH). The Hansen solubility parameter is in units of MPa 0.5 or (J/cc) 0.5.
δD: Energy derived from intermolecular dispersion force
δP: Energy derived from intermolecular polar force
δH: Energy derived from intermolecular hydrogen bonding force

These three parameters (that is, δD, δP, and δH) are plotted as coordinates for points in three dimensions, also known as the Hansen space.

Within this three-dimensional space (Hansen space), the closer two molecules are, the more likely the two molecules are to dissolve into each other. In order to evaluate whether two molecules (molecules (1) and (2)) come closer to each other in the Hansen space, the distance (Ra) between the interactions of Hansen solubility parameters is determined. Ra is calculated by the following Formula.

$${( Ra )}^{2} = 4 {( {δ}_{d 2} - {δ}_{d 1} )}^{2} + {( {δ}_{p 2} - {δ}_{p 1} )}^{2} + {( {δ}_{h 2} - {δ}_{h 1} )}^{2}$$

[In the equation shown above, δ_{d1}, δₚ₁, and δₕ₁ each represent δD, δP, and δH of the molecule (1). δ_{d2}, δₚ₂, and δₕ₂ each represent δD, δP, and δH of the molecule (2).]

The Hansen solubility parameters of the compound in which the anion moiety of the component (D0) is protonated and the water can be calculated with “Molecular Modeling Pro” software, version 5.1.9 (ChemSW, Fairfield CA, www.chemsw.com). Hansen Solubility from Dynacomp Software, or the like.

In the present embodiment, preferred examples of the component (D0) include a cation in which a cation moiety ((M^{m+})_{1/m}) is represented by General Formula (ca-1); and an anion moiety which is a compound in which “ring structure” is bonded to a (t+2)-valent aromatic hydrocarbon group as R^{Ar}.

That is, suitable examples of the component (D0) include a compound in which “(M^{m+})_{1/m}” in General Formula (d0) represents a cation represented by General Formula (ca-1) and two or more of R⁰¹, R⁰², and R⁰³ in General Formula (d0) are bonded to each other to form a ring structure (here, in a case where two of R⁰¹, R⁰², and R⁰³ are bonded to each other to form a ring structure, the ring structure is an alicyclic ring, and the remaining one represents a chain-like hydrocarbon group or a hydrogen atom. The ring structure may have a substituent. The chain-like hydrocarbon group may have a substituent).

In the component (D0) having the ring structure, the distance (Ra) between the interactions is preferably 32 or greater, more preferably 32 or greater and 36 or less, still more preferably 32.5 or greater and 35.5 or less, and particularly preferably 33 or greater and 35.5 or less.

Suitable examples of the component (D0) are shown below. Ra represents the distance (Ra) between the interactions of the Hansen solubility parameter of the compound in which the anion moiety is protonated and the Hansen solubility parameter of water.

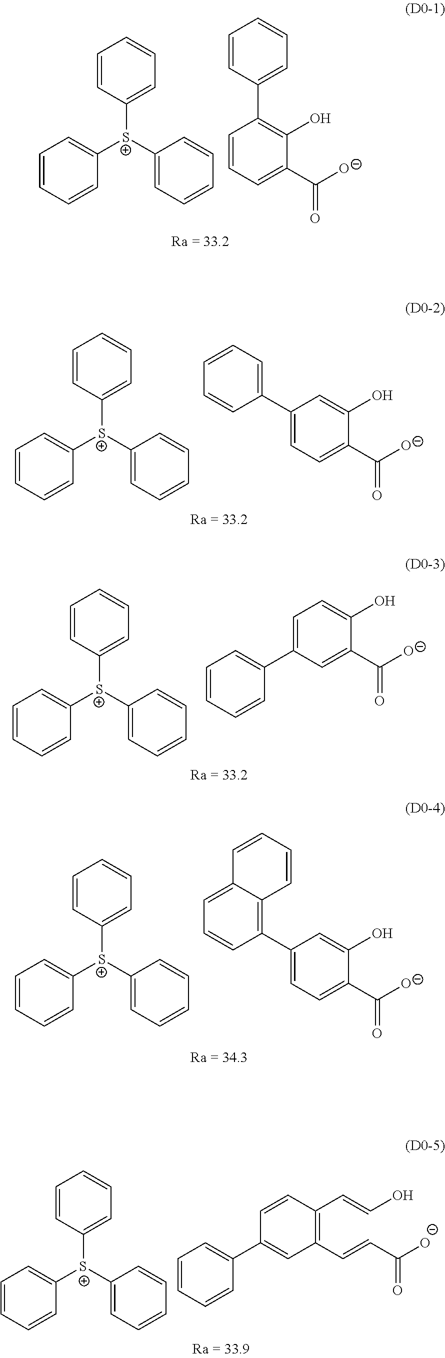

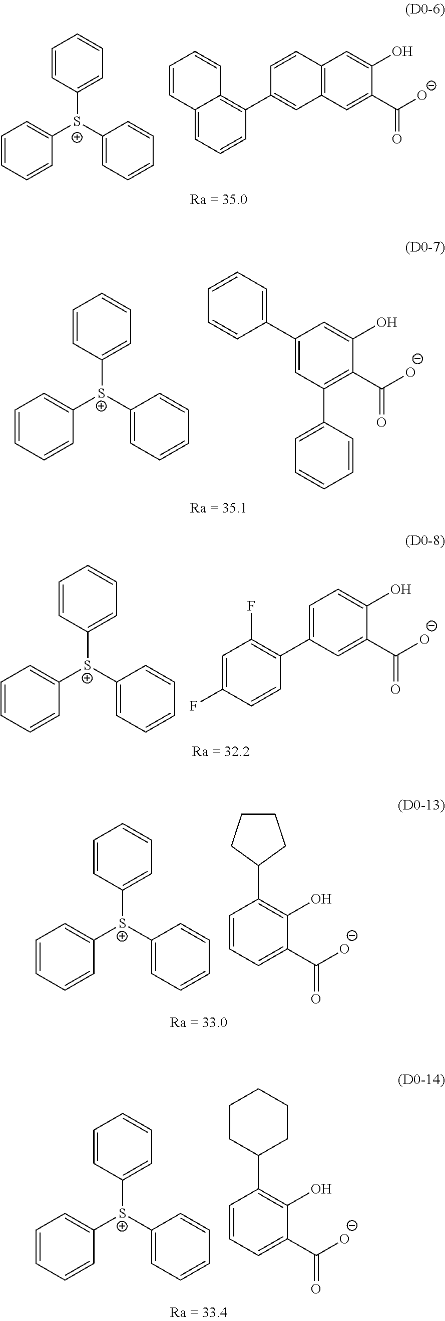

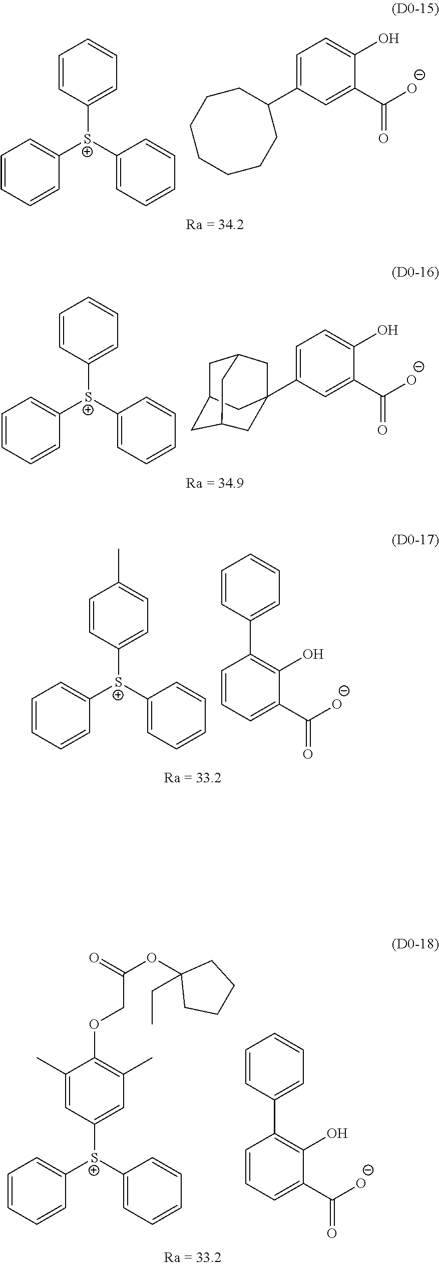

Alternatively, preferred examples of the component (D0) include a cation in which a cation moiety ((M^{m+})_{1/m}) is represented by General Formula (ca-1); and an anion moiety which is a compound in which “chain-like hydrocarbon group” is bonded to a (t+2)-valent aromatic hydrocarbon group as R^{Ar}.

That is, suitable examples of the component (D0) include a compound in which “(M^{m+})_{1/m}” in General Formula (d0) represents a cation represented by General Formula (ca-1) and R⁰¹ and R⁰² in General Formula (d0) each independently represent a chain-like hydrocarbon group, and R⁰³ represents a chain-like hydrocarbon group or a hydrogen atom (here, the chain-like hydrocarbon group may have a substituent).

In the component (D0) containing the chain-like hydrocarbon group, the distance (Ra) between the interactions is preferably 32 or greater, more preferably 32 or greater and 37.5 or less, still more preferably 32.5 or greater and 37 or less, and particularly preferably 33 or greater and 37 or less.

Suitable examples of the component (D0) are shown below. Ra represents the distance (Ra) between the interactions of the Hansen solubility parameter of the compound in which the anion moiety is protonated and the Hansen solubility parameter of water.

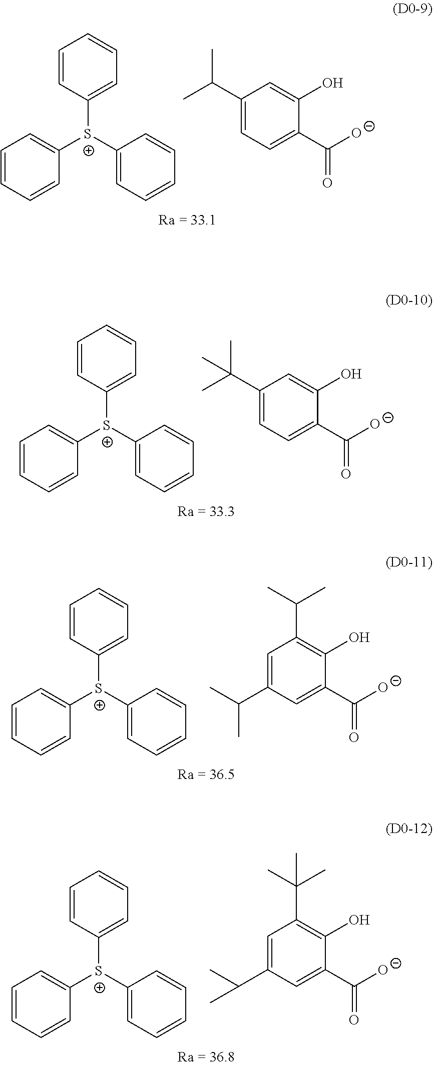

Among the examples, from the viewpoint of easily enhancing the roughness characteristics and the effect of suppressing occurrence of defects, suitable examples of the component (D)) include a compound in which “(M^{m+})_{1/m}” in General Formula (d0) represents a cation represented by General Formula (ca-1) and two or more of R⁰¹, R⁰², and R⁰³ in General Formula (d0) are bonded to each other to form a ring structure (here, in a case where two of R⁰¹, R⁰², and R⁰³ are bonded to each other to form a ring structure, the ring structure is an alicyclic ring, and the remaining one represents a chain-like hydrocarbon group or a hydrogen atom. The ring structure may have a substituent. The chain-like hydrocarbon group may have a substituent).

In the resist composition according to the present embodiment, the component (D0) may be used alone or in a combination of two or more kinds thereof.

The content of the component (D0) in the resist composition according to the present embodiment is preferably in a range of 0.5 to 20 parts by mass, more preferably in a range of 0.5 to 15 parts by mass, and still more preferably in a range of 1 to 10 parts by mass with respect to 100 parts by mass of the component (A).

In a case where the content of the component (D0) is greater than or equal to the lower limits of the above-described preferable ranges, the lithography characteristics such as resolution, roughness characteristics, and suppression of occurrence of defects, and a resist pattern shape are likely to be obtained satisfactorily. Meanwhile, in a case where the content thereof is less than or equal to the upper limits of the above-described preferable ranges, the sensitivity can be satisfactorily maintained, and the throughput is also excellent.

### <Other Components>

The resist composition according to the present embodiment may further contain other components in addition to the component (A) and the component (D0) described above. Examples of the other components include a component (B), a component (D), a component (E), a component (F), and a component (S), which are described below.

### <<Acid Generator Component (B)>>

It is preferable that the resist composition according to the present embodiment further contains an acid generator component (B) which generates an acid upon light exposure in addition to the component (A) and the component (D0).

The component (B) is not particularly limited, and those which have been suggested so far as an acid generator for a chemically amplified resist composition in the related art can be used.

Examples of the acid generation agent include various acid generation agents, for example, onium salt-based acid generation agents such as iodonium salts and sulfonium salts; oxime sulfonate-based acid generation agents; diazomethane-based acid generation agents such as bisalkyl or bisaryl sulfonyl diazomethanes and poly(bis-sulfonyl)diazomethanes; nitrobenzyl sulfonate-based acid generation agents, iminosulfonate-based acid generation agents, and disulfone-based acid generation agents.

Examples of the onium salt-based acid generation agents include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

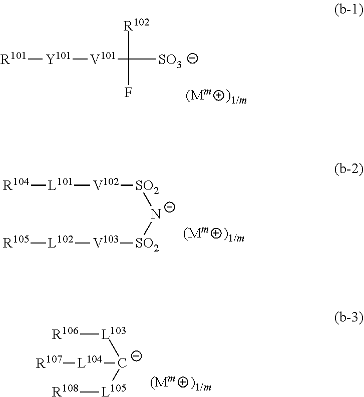

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings are substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and other heterocyclic groups each represented by Chemical Formulae (r-hr-1) to (r-hr-16). In the formulae, * represents a bonding site with respect to Y¹⁰¹ in Formula (b-1).

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group having a condensed ring in which an aliphatic hydrocarbon ring and an aromatic ring are condensed. Examples of the condensed ring include those obtained by condensing one or more aromatic rings with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site with respect to Y¹⁰¹ in Formula (b-1).

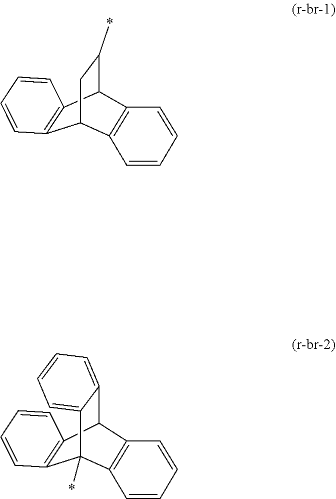 [SMILES: *C1CC2c3ccccc3C1c1ccccc12 ; *C12c3ccccc3C(c3ccccc31)c1ccccc12]

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclo[5.2.1.0^{2,6}]decane, or tetracyclododecane; a lactone-containing cyclic group represented by any of General Formulae (a2-r-1) to (a2-r-7); a —SO₂-containing cyclic group represented by any of General Formulae (a5-r-1) to (a5-r-4); and a heterocyclic group represented by any of Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent. More specifically, as the cyclic hydrocarbon group, a phenyl group, a naphthyl group, or a group in which one or more hydrogen atoms have been removed from a polycycloalkane, a lactone-containing cyclic group represented by any of General Formulae (a2-r-1) to (a2-r-7), or a —SO₂-containing cyclic group represented by any of General Formulae (a5-r-1) to (a5-r-4) is preferable, a group in which one or more hydrogen atoms have been removed from a polycycloalkane or a —SO₂-containing cyclic group represented by any of General Formulae (a5-r-1) to (a5-r-4) is more preferable, and an adamantyl group or a —SO₂-containing cyclic group represented by General Formula (a5-r-1) is still more preferable.

In a case where the cyclic hydrocarbon group has a substituent, it is preferable that the substituent is a hydroxyl group.

In Formula (b-1). Y¹⁰¹ represents a single bond or a divalent linking group having an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of the divalent linking group having an oxygen atom include linking groups each represented by General Formulae (y-al-1) to (y-al-7). Further, in General Formulae (y-al-1) to (y-al-7), V′¹⁰¹ in General Formulae (y-al-1) to (y-al-7) is bonded to R¹⁰¹ in Formula (b-1).

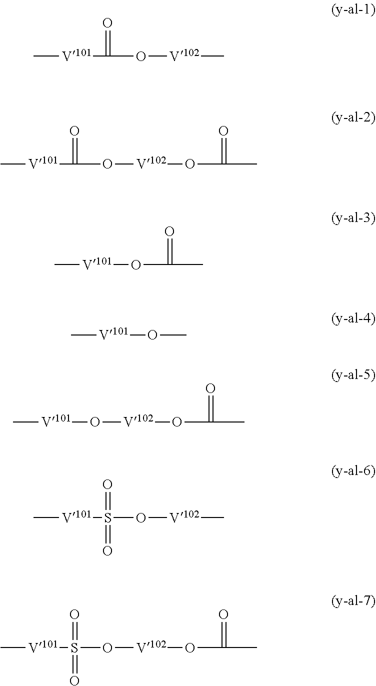

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

Y¹⁰¹ represents preferably a divalent linking group having an ester bond or a divalent linking group having an ether bond and more preferably a linking group represented by any of Formulae (y-al-1) to (y-al-5).

In Formula (b-1). V¹⁰¹ represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among the examples, it is preferable that V¹⁰¹ represents a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

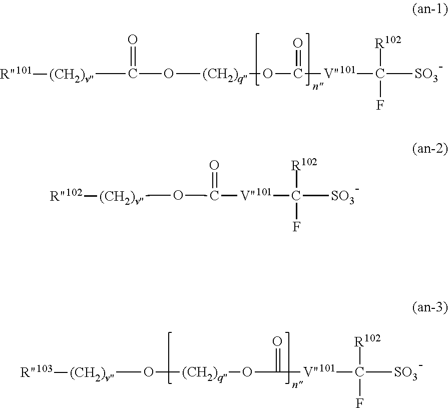

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (a5-r-1) to (a5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R″¹⁰³, the same groups as those for the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R105 decreases within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3). L¹⁰³ to L¹⁰⁵ each independently represent a single bond, —CO—, or —SO₂—.

Among the examples, as the anion moiety of the component (B), an anion in the component (b-1) is preferable. Among these, an anion represented by any one of General Formulae (an-1) to (an-3) is more preferable, and an anion represented by any one of General Formula (an-1) or (an-2) is still more preferable.

### {Cation Moiety}

In Formulae (b-1), (b-2), and (b-3), in represents an integer of 1 or greater. M^{m+} represents an m-valent onium cation and suitably a sulfonium cation or an iodonium cation, and examples thereof include organic cations each represented by General Formulae (ca-1) to (ca-3).

Specific examples of the suitable cation represented by Formula (ca-1) include cations each represented by Formulae (ca-1-1) and (ca-1-70).

Specific examples of suitable cations represented by Formula (ca-2) include a diphenyliodonium cation and a bis(4-tert-butylphenyl)iodonium cation.

Specific examples of the suitable cations each represented by Formula (ca-3) include cations each represented by Formulae (ca-3-1) and (ca-3-6).

Among the examples, a cation represented by General Formula (ca-1) is preferable as the cation moiety ((M^{m+})_{1/m}).

In the resist composition according to the present embodiment, the component (B) may be used alone or in combination of two or more kinds thereof.

The content of the component (B) in the resist composition of the present embodiment is preferably less than 40 parts by mass, more preferably in a range of 1 to 30 parts by mass, and still more preferably in a range of 3 to 25 parts by mass with respect to 100 parts by mass of the component (A).

In a case where the content of the component (B) is set to be in the above-described preferable range, pattern formation can be satisfactorily performed. Further, it is preferable that each component of the resist composition is dissolved in an organic solvent from the viewpoint that a uniform solution is easily obtained and the storage stability of the resist composition is improved.

### <<Base Component (D)>>

The resist composition according to the present embodiment may further contain, in addition to the components (A) and (D0), a base component (component (D)) that traps (that is, controls the diffusion of an acid) an acid generated upon light exposure. The component (D) acts as a quencher (an acid diffusion control agent) which traps the acid generated in the resist composition upon light exposure.

Examples of the component (D) include a photodecomposable base (D1) that is decomposed upon light exposure and loses acid diffusion controllability (hereinafter, referred to as “component (D1)”, and a component corresponding to the component (D0) is excluded) and a nitrogen-containing organic compound (D2) not corresponding to the component (D0) and the component (D1) (hereinafter, referred to as “component (D2)”).

### In Regard to Component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

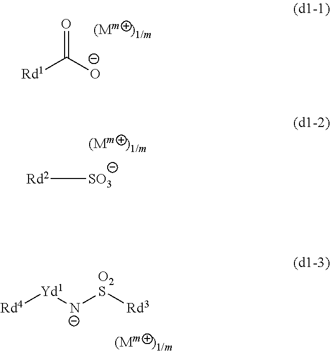

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Yd¹ represents a single bond or a divalent linking group. m represents an integer of 1 or greater, and M^{m+}'s each independently represent an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-al-1) to (y-al-5) is preferable as the substituent. Further, in a case where the aromatic hydrocarbon group, the aliphatic cyclic group, or the chain-like alkyl group as Rd¹ contain a linking group represented by any of General Formulae (y-al-1) to (y-al-7) as a substituent, V′¹⁰¹ in General Formulae (y-al-1) to (y-al-7) is bonded to the carbon atom constituting the aromatic hydrocarbon group, the aliphatic cyclic group, or the chain-like alkyl group as Rd¹ in Formula (d3-1), in General Formulae (y-al-1) to (y-al-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclo[5.2.1.0^{2,6}]decane, or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

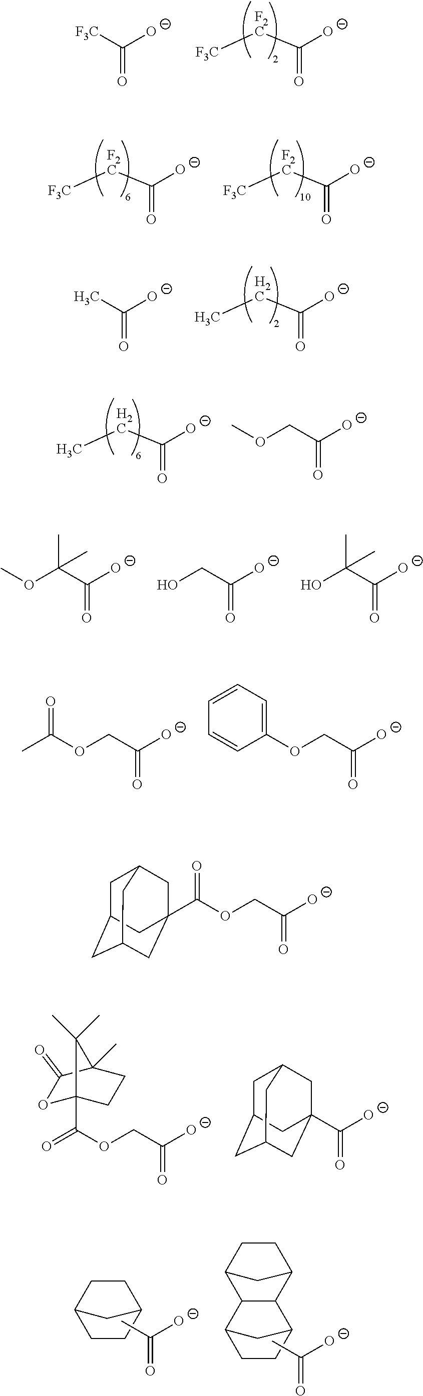

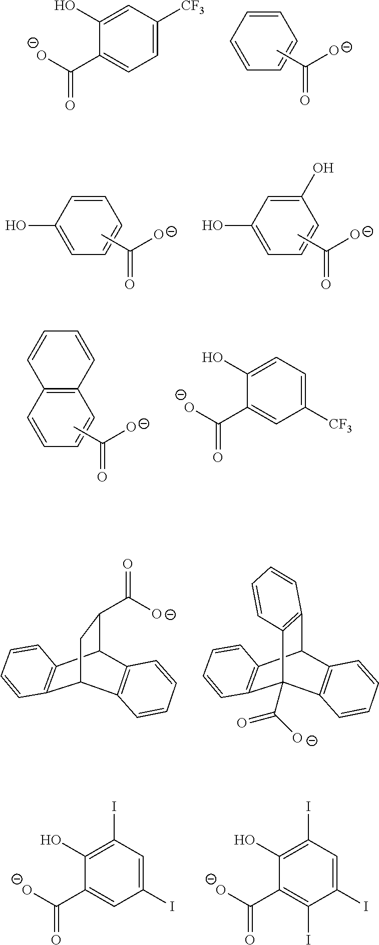

### Cation Moiety

In Formula (d1-1), M^{m+} represents an m-valent organic cation.

Suitable examples of the organic cation as M^{m+} include the same cations as those each represented by General Formulae (ca-1) to (ca-3). Among these, a cation represented by General Formula (ca-1) is more preferable, and a cation represented by any of Formulae (ca-1-1) to (ca-1-70) is still more preferable.

The component (d1-1) may be used alone or in combination of two or more kinds thereof.

{Component (d1-2)}

### Anion Moiety

In Formula (d1-2). Rd² represents a cyclic group which may have a substituent, a chain-like alkyl group which may have a substituent, or a chain-like alkenyl group which may have a substituent, and examples thereof include the same groups as those for R′²⁰¹.

Here, no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

It is preferable that Rd² represents a chain-like alkyl group which may have a substituent or an aliphatic cyclic group which may have a substituent. The chain-like alkyl group has preferably 1 to 10 carbon atoms and more preferably 3 to 10 carbon atoms. As the aliphatic cyclic group, a group in which one or more hydrogen atoms have been removed from adamantane, norbornane, isobornane, tricyclo[5.2.1.0^{2,6}]decane, or tetracyclododecane (a group which may have a substituent); and a group in which one or more hydrogen atoms have been removed from camphor or the like are more preferable.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

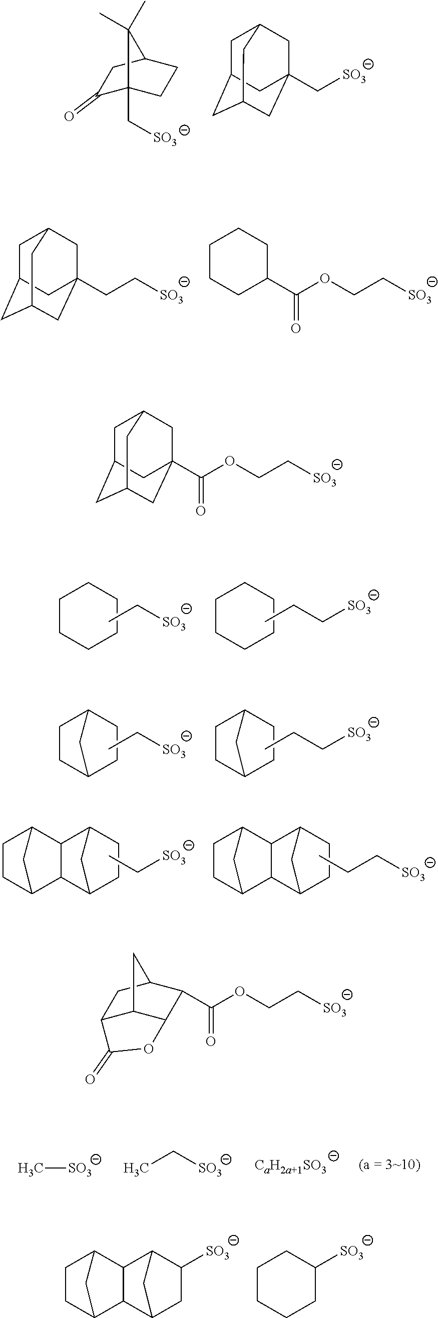

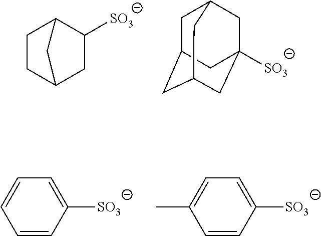

### Cation Moiety

In Formula (d1-2). M^{m+} represents an m-valent organic cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclo[5.2.1.0^{2,6}]decane, or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. In addition, in a case where Rd4 represents an aromatic group, the resist composition has excellent light absorption efficiency and satisfactory sensitivity and lithography characteristics in the lithography.

In Formula (d1-3). Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. Examples of the divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

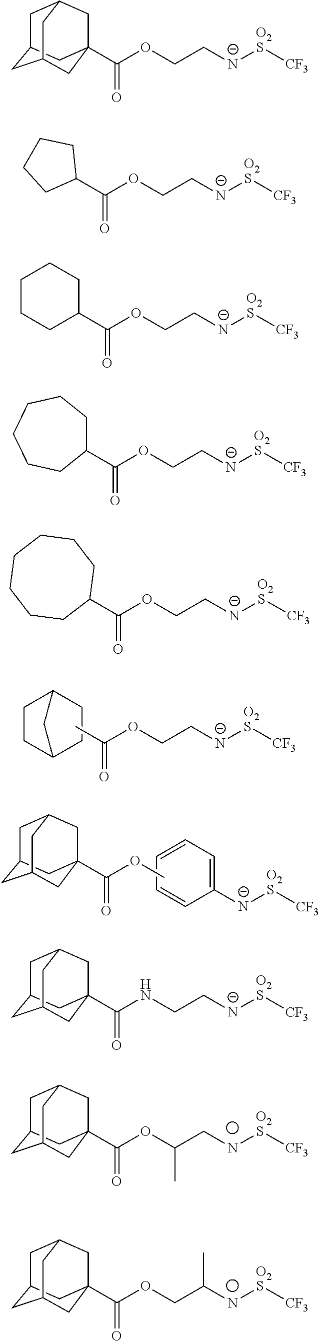

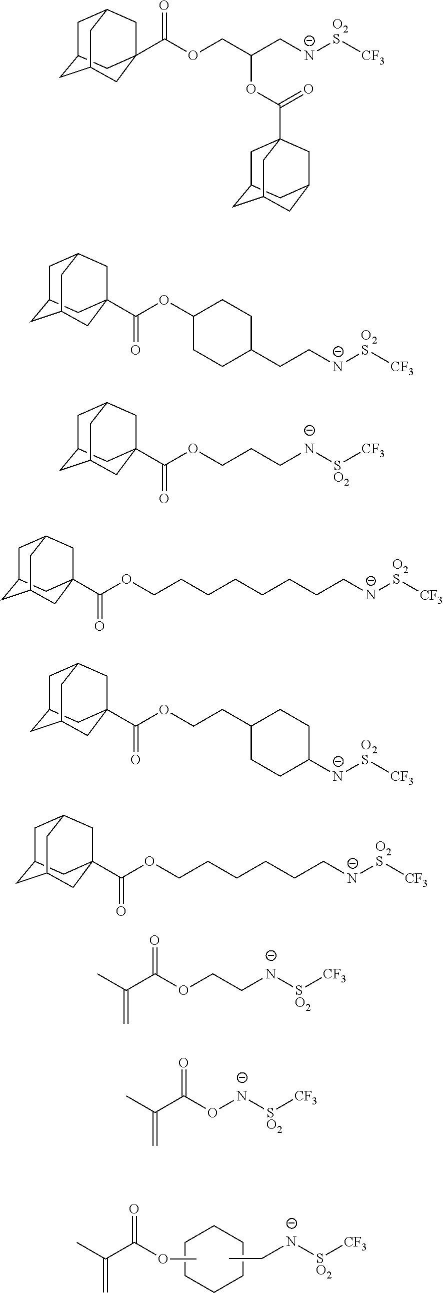

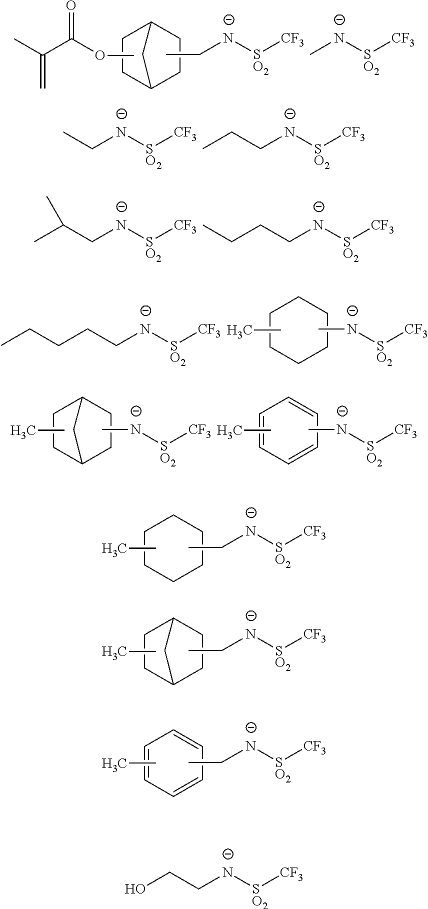

### Cation Moiety

In Formula (d1-3). M^{m+} represents an m-valent organic cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 2 to 8 parts by mass with respect to 100 parts by mass of the component (A).

In a case where the content of the component (D1) is greater than or equal to the lower limits of the above-described preferable ranges, satisfactory lithography characteristics and a satisfactory resist pattern shape are likely to be obtained. On the contrary, in a case where the content is less than or equal to the upper limits of the above-described ranges, the sensitivity can be satisfactorily maintained and the throughput is also excellent.

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component acts as an acid diffusion control agent and corresponds to neither the component (D0) nor the component (D1), and any known component of the related art may be used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1, 5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy]ethyl}amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

The component (D2) may be used alone or in combination of two or more kinds thereof.

In a case where the resist composition contains the component (D2), the content of the component (D2) in the resist composition is typically in a range of 0.01 to 5 parts by mass with respect to 100 parts by mass of the component (A). In a case where the content thereof is set to be in the above-described range, the resist pattern shape, the post exposure temporal stability, and the like are improved.

<<At Least One Compound (F) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described range, the sensitivity, lithography characteristics, and the like are improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application. First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

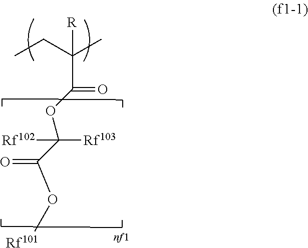

[in the formula. R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms are substituted with halogen atoms. Among these, a fluorine atom is preferable as the halogen atom. Among these. Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1). nf¹ represents an integer of 0 to 5, preferably an integer of 0 to 3, and more preferably 1 or 2.

In Formula (f1-1). Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition of the present embodiment can be produced by dissolving the resist materials in an organic solvent component (hereinafter, referred to as “component (S)”).

The component (S) may be any organic solvent which can dissolve each component to be used to obtain a uniform solution, and an optional organic solvent can be appropriately selected from those which have been known as solvents of a chemically amplified resist composition and then used.

Examples of the component (S) include lactones such as γ-butyrolactone; ketones such as acetone, methyl ethyl ketone, cyclohexanone, methyl-n-pentyl ketone, methyl isopentyl ketone, and 2-heptanone: polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition of the present embodiment, the component (S) may be used alone or in the form of a mixed solvent of two or more kinds thereof. Among these, PGMEA, PGME, γ-butyrolactone. EL, or cyclohexanone is preferable.

Further, a mixed solvent obtained by mixing PGMEA with a polar solvent is also preferable as the component (S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

Further, a mixed solvent of 7-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

After the resist material is dissolved in the component (S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the compound (D0) represented by General Formula (d0) as the base component. Since the component (D0) is a photodecomposable base which generates an acid having a high pKa, the effect of controlling acid diffusion can be enhanced. In addition, since the anion moiety has a specific structure, that is, a structure in which a functional group (a ring structure or a branched hydrocarbon group) having 3 or more carbon atoms is bonded to an aromatic hydrocarbon group, the hydrophobicity is increased. Therefore, according to the resist composition containing such a component (D0), both the roughness characteristics and the effect of suppressing the occurrence of defects are considered to be enhanced.

### (Resist Pattern Forming Method)

A resist pattern forming method according to a second aspect of the present invention is a method including a step of forming a resist film on a support using the resist composition according to the first aspect of the present invention described above, a step of performing liquid immersion exposure on the resist film, and a step of developing the resist film exposed to light to form a resist pattern.

According to the embodiment of the resist pattern forming method, a resist pattern forming method by performing processes as described below is an exemplary example.

First, a support is coated with the resist composition of the above-described embodiment using a spinner or the like, and a pre-bake (post applied bake (PAB)) treatment is performed under a temperature condition of 80° C. to 150° C. for 40 to 120 seconds and preferably 60 to 90 seconds to form a resist film.

Next, the resist film is subjected to liquid immersion exposure. The resist film is selectively exposed to light through a mask (mask pattern) on which a predetermined pattern is formed using, for example, an exposure apparatus such as an ArF exposure apparatus, and is subjected to a bake (post-exposure bake (PEB)) treatment, for example, under a temperature condition of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength used for light exposure is not particularly limited and the light exposure can be conducted using radiation such as an ArF excimer laser, a KrF excimer laser, an F2 excimer laser, extreme ultraviolet rays (EUV), vacuum ultraviolet rays (VUV), electron beams (EB), X-rays, and soft X-rays. The resist composition is highly useful for a KrF excimer laser, an ArF excimer laser, EB, or EUV, and is more useful for an ArF excimer laser.

The resist pattern forming method according to the present embodiment is a particularly useful method in a case where the method of exposing the resist film to light is liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a larger refractive index than the refractive index of air, and the light exposure (immersion exposure) is performed in this state.

The liquid immersion medium is preferably a solvent that exhibits a refractive index greater than the refractive index of air but less than the refractive index of the resist film to be exposed, and examples thereof include water, a fluorine-based inert liquid, a silicon-based solvent, and a hydrocarbon-based solvent. As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

The ketone-based solvent is an organic solvent containing C—C(═O)—C in the structure thereof. The ester-based solvent is an organic solvent containing C—C(═O)—O—C in the structure thereof. The alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. The nitrile-based solvent is an organic solvent containing a nitrile group in the structure thereof. The amide-based solvent is an organic solvent containing an amide group in the structure thereof. The ether-based solvent is an organic solvent containing C—O—C in the structure thereof.

Some organic solvents have a plurality of the functional groups which characterize each of the solvents in the structure thereof. In such a case, the organic solvents are considered to correspond to all the solvents containing the functional groups. For example, diethylene glycol monomethylether corresponds to both the alcohol-based solvent and the ether-based solvent which have been classified above.

The hydrocarbon-based solvent is a hydrocarbon solvent which is formed of a hydrocarbon that may be halogenated and does not have a substituent other than halogen atoms. Among these, a fluorine atom is preferable as the halogen atom.

Among the examples, as the organic solvent contained in the organic developing solution, a polar solvent is preferable. Further, a ketone-based solvent, an ester-based solvent, and a nitrile-based solvent are preferable.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone, and methyl amyl ketone (2-heptanone). Among these examples, methyl amyl ketone (2-heptanone) is preferable as the ketone-based solvent.

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these examples, butyl acetate is preferable as the ester-based solvent.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method for immersing a support in a developing solution for a certain time (a dip method), a method for raising a developing solution on the surface of a support using the surface tension and maintaining the state for a certain time (a puddle method), a method for spraying a developing solution to the surface of a support (spray method), and a method for continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in combination of two or more kinds thereof. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method for performing the rinse treatment include a method for continuously ejecting a rinse solution onto a support rotating at a certain rate (rotary coating method), a method for immersing a support in a rinse solution for a certain time (dip method), and a method for spraying a rinse solution to the surface of a support (spray method).

According to the resist pattern forming method of the present embodiment described above, since the above-described resist composition is used, the occurrence of defects is further suppressed, and in addition, a resist pattern having a satisfactory shape in which the roughness is further reduced can be formed.

(Compound Represented by General Formula (d0))

The compound according to a third aspect of the present invention is a compound represented by General Formula (d0).

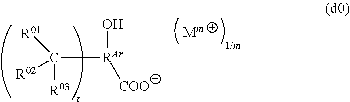

[in the formula, R^{Ar} represents a (t+2)-valent aromatic hydrocarbon group which may have a substituent. t represents an integer of 1 or greater. R⁰¹ and R⁰² each independently represent a chain-like hydrocarbon group, R⁰³ represents a chain-like hydrocarbon group or a hydrogen atom, or two or more of R⁰¹, R⁰², and R⁰³ are bonded to each other to form a ring structure. Here, in a case where two of R⁰¹, R⁰², and R⁰³ are bonded to each other to form a ring structure, the ring structure is an alicyclic ring, and the remaining one represents a chain-like hydrocarbon group or a hydrogen atom. The chain-like hydrocarbon group may have a substituent. The ring structure may have a substituent. m represents an integer of 1 or greater, and M^{m+} represents an m-valent organic cation.]

The compound according to the third aspect is the same compound as the component (D0) described in the explanation of the resist composition according to the first aspect. R^{Ar}, t, R⁰¹, R⁰², R⁰³, m, and M^{m+} in Formula (d0) each have the same definition as described above.

Suitable examples of the compound represented by General Formula (d0) include a compound in which “(M^{m+})_{1/m}” in General Formula (d0) represents a cation represented by General Formula (ca-1) and two or more of R⁰¹, R⁰², and R⁰³ in General Formula (d0) are bonded to each other to form a ring structure (here, in a case where two of R⁰¹, R⁰², and R⁰³ are bonded to each other to form a ring structure, the ring structure is an alicyclic ring, and the remaining one represents a chain-like hydrocarbon group or a hydrogen atom. The ring structure may have a substituent. The chain-like hydrocarbon group may have a substituent).

In the compound represented by General Formula (d0) which has the ring structure, the distance (Ra) between the interactions is preferably 32 or greater, more preferably 32 or greater and 36 or less, still more preferably 32.5 or greater and 35.5 or less, and particularly preferably 33 or greater and 35.5 or less.

Further, the distance (Ra) between the interactions represents the distance between the interactions of the Hansen solubility parameter of the compound in which the anion moiety is protonated and the Hansen solubility parameter of water.

Examples of the compound represented by General Formula (d0) which has the ring structure include a compound (D0-1), a compound (D0-2), a compound (D0-3), a compound (D0-4), a compound (D0-5), a compound (D0-6), a compound (D0-7), a compound (D0-8), a compound (D0-13), a compound (D0-14), a compound (D0-15), a compound (D0-16), a compound (D0-17), and a compound (D0-18) described above.

Alternatively, suitable examples of the compound represented by General Formula (d0) include a compound in which “(M^{m+})_{1/m}” in General Formula (d0) represents a cation represented by General Formula (ca-1) and R⁰¹ and R⁰² in General Formula (d0) each independently represent a chain-like hydrocarbon group, and R⁰³ represents a chain-like hydrocarbon group or a hydrogen atom (here, the chain-like hydrocarbon group may have a substituent).

In the compound represented by General Formula (d0) which contains the chain-like hydrocarbon group, the distance (Ra) between the interactions is preferably 32 or greater, more preferably 32 or greater and 37.5 or less, still more preferably 32.5 or greater and 37 or less, and particularly preferably 33 or greater and 37 or less.

Examples of the compound represented by General Formula (d0) which contains the chain-like hydrocarbon group include a compound (D0-9), a compound (D0-10), a compound (D0-11), and a compound (D0-12) described above.

[Method of Producing Compound Represented by General Formula (d0)]

The component (D0) is produced, for example, as follows. That is, the compound (d0-0) represented by General Formula (d0-0) and the salt exchange compound (Xh⁻(M^{m+})_{1/m}) represented by the following chemical formula are subjected to a salt exchange reaction in the presence of an appropriate base, and thus a compound represented by General Formula (d0) can be produced.

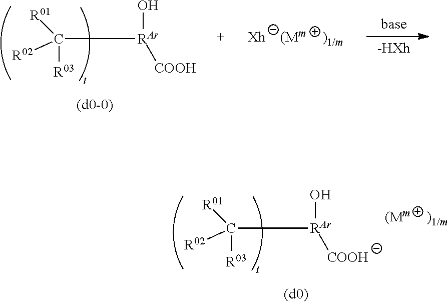

[In the formula, R^{Ar} represents a (t+2)-valent aromatic hydrocarbon group which may have a substituent. t represents an integer of 1 or greater. R⁰¹ and R⁰² each independently represent a chain-like hydrocarbon group, R⁰; represents a chain-like hydrocarbon group or a hydrogen atom, or two or more of R⁰¹, R⁰², and R⁰³ are bonded to each other to form a ring structure. Here, in a case where two of R⁰¹, R⁰², and R⁰³ are bonded to each other to form a ring structure, the ring structure is an alicyclic ring, and the remaining one represents a chain-like hydrocarbon group or a hydrogen atom. The chain-like hydrocarbon group may have a substituent. The ring structure may have a substituent. Xh⁻ represents a halogen ion or an alkylsulfonate ion. m represents an integer of 1 or greater, and M^{m+} represents an m-valent organic cation.]

In the reaction formula shown above, examples of the halogen atom constituting the halogen ion as Xh⁻ include a fluorine atom, a chlorine atom, a bromine atom, and an iodine atom. Among these, a fluorine atom is preferable.

An alkyl group in the alkylsulfonate ion may be linear, branched, or cyclic. The linear or branched alkyl group is preferably an alkyl group having 1 to 10 carbon atoms, more preferably an alkyl group having 1 to 8 carbon atoms, and still more preferably an alkyl group having 1 to 4 carbon atoms, and particularly preferably a methyl group or an ethyl group. The number of carbon atoms in the cyclic alkyl group is preferably in a range of 4 to 15, more preferably in a range of 4 to 10, and still more preferably in a range of 6 to 10.

Examples of the base include organic bases such as triethylamine, 4-dimethylaminopyridine (DMAP), and pyridine; and inorganic bases such as sodium hydride, K₂CO₃, and Cs₂CO₃.

The salt exchange between the compound (d0-0) and the organic cation (M^{m+}) can be carried out by the same method as a known salt substitution method of the related art. For example, the compound (d0-0) and the salt exchange compound (Xh⁻(M^{m+})_{1/m}) are allowed to react by being stirred or the like in a solvent such as water, dichloromethane, acetonitrile, or chloroform to carry out the salt exchange.

The reaction temperature is preferably about 0° C. to 100° C. and more preferably about 0° C. to 50° C.

The reaction time varies depending on the reactivity of the compound (d0-0) with the salt exchange compound, the reaction temperature, and the like, but is typically preferably 10 minutes or longer and 24 hours or shorter and more preferably 10 minutes or longer and 12 hours or shorter.

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), an elemental analysis method, and an X-ray crystal diffraction method.

### (Acid Diffusion Control Agent)

An acid diffusion control agent according to a fourth aspect of the present invention contains the compound according to the third aspect described above.

Such an acid diffusion control agent is useful as a quencher for a chemically amplified resist composition, for example, a quencher for the resist composition according to the first aspect described above. In a case where such an acid diffusion control agent is used in a chemically amplified resist composition, the resolution is enhanced, occurrence of defects is suppressed, and a fine pattern having a satisfactory shape in which roughness of the resist pattern is further reduced can be easily formed.

### EXAMPLES

Hereinafter, the present invention will be described in more detail based on examples, but the present invention is not limited to these examples.

In the present examples, a compound represented by Chemical Formula (1) is denoted by “compound (1)”, and compounds represented by other chemical formulae are also denoted in the same manner.

### <Production of Compounds>

### [Production of Compound (D0-1)]

3-phenylsalicylic acid (5 g), dichloromethane (100 g), and triphenylsulfonium bromide (8 g) were mixed with each other. Thereafter, triethylamine (2.36 g) was added dropwise thereto, and the mixture was stirred at room temperature for 30 minutes to obtain a reaction solution.

Next, ion exchange water (100 g) was added to the reaction solution, and the mixture was stirred at room temperature for 30 minutes, and the organic phase was separated (water washing operation). This water washing operation was repeated 12 times.

Next, the obtained organic phase was concentrated, thereby obtaining a compound (9.4 g) represented by Chemical Formula (D0-1).

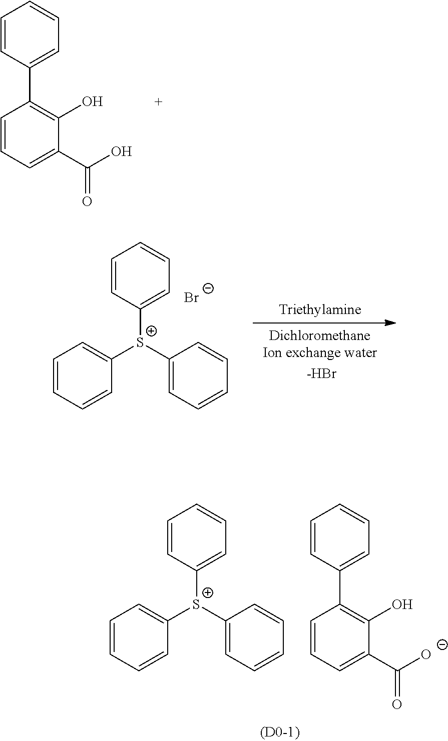 [SMILES: O=C(O)c1cccc(-c2ccccc2)c1O ; c1ccc([S+](c2ccccc2)c2ccccc2)cc1 ; [Br-] ; "-HBr" ; c1ccc([S+](c2ccccc2)c2ccccc2)cc1 ; O=C([O-])c1cccc(-c2ccccc2)c1O]

### [Production of Compounds (D0-2) to (D0-18)]

Each compound represented by Chemical Formulae (D0-2) to (D0-18) was obtained in the same manner as in “production of compound (D0-1)” except that 3-phenylsalicylic acid was changed to each of the corresponding raw material compounds.

NMR measurement was performed on each of the obtained compounds (D0-1) to (D0-18), and the structure of each compound was identified from the measurement results.

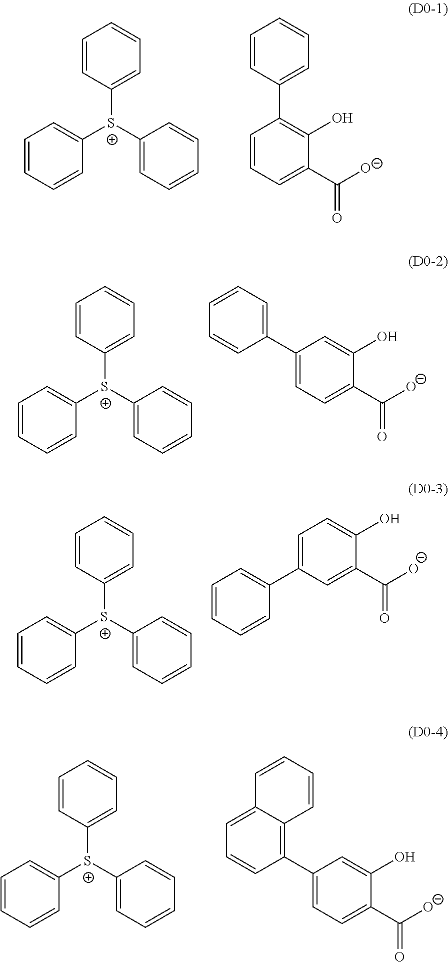

Compound (D0-1): ¹H-NMR (DMSO-d6) δ (ppm)=7.89-7.60 (m, 18H), 7.35 (d, 2H), 7.22 (d, 2H), 6.62 (m, 1H)

Compound (D0-2): ¹H-NMR (DMSO-d6) δ (ppm)=7.90-7.52 (m, 17H), 7.44 to 7.20 (m, 4H), 6.66 (m, 2H)

Compound (D0-3): ¹H-NMR (DMSO-d6) δ (ppm)=7.89 to 7.60 (m, 18H), 7.42 to 7.24 (m, 4H), 6.45 (m, 1H)

Compound (D0-4): ¹H-NMR (DMSO-d6) δ (ppm)=7.92-7.18 (m, 23H), 6.70 (m, 2H)

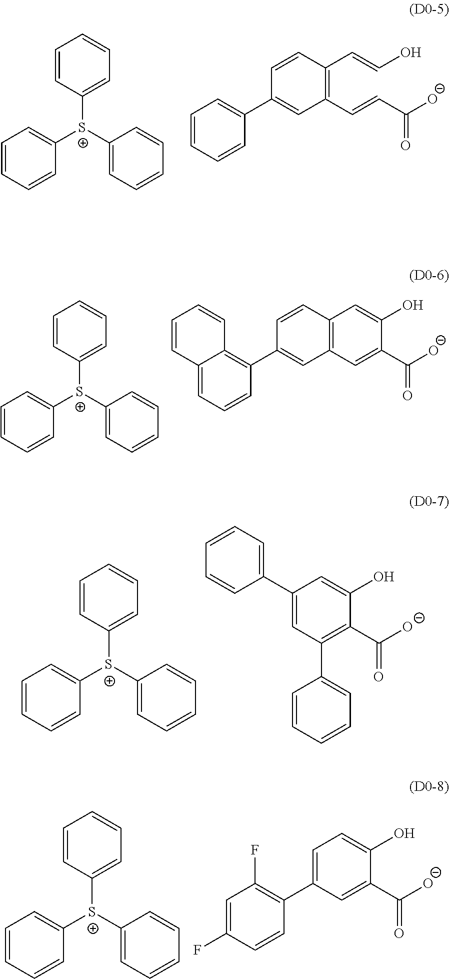

Compound (D0-5): ¹H-NMR (DMSO-d6) δ (ppm)=8.26 (t, 1H), 7.89-7.55 (m, 21H), 7.31 (t, 1H), 7.13 (t, 1H), 6.91 (s, 1H)

Compound (D0-6): ¹H-NMR (DMSO-d6) δ (ppm)=8.60 (t, 1H), 8.19 (dd, 1H), 8.11-7.96 (m, 2H), 7.85-7.51 (m, 20H), 7.28 (t, 1H), 7.10 (t, 1H), 6.87 (s, 1H)

Compound (D0-7): ¹H-NMR (DMSO-d6) δ (ppm)=7.86-7.35 (m, 26H), 6.80 (d, 1H)

### Compound (D0-8):

¹H-NMR (DMSO-d6) δ (ppm)=8.10 (d, 1H), 7.82-7.35 (m, 19H), 6.52 (d, 1H)

¹⁹F-NMR (DMSO-d6) δ (ppm)=−113.5 (s, 1F), −112.2 (s, 1F)

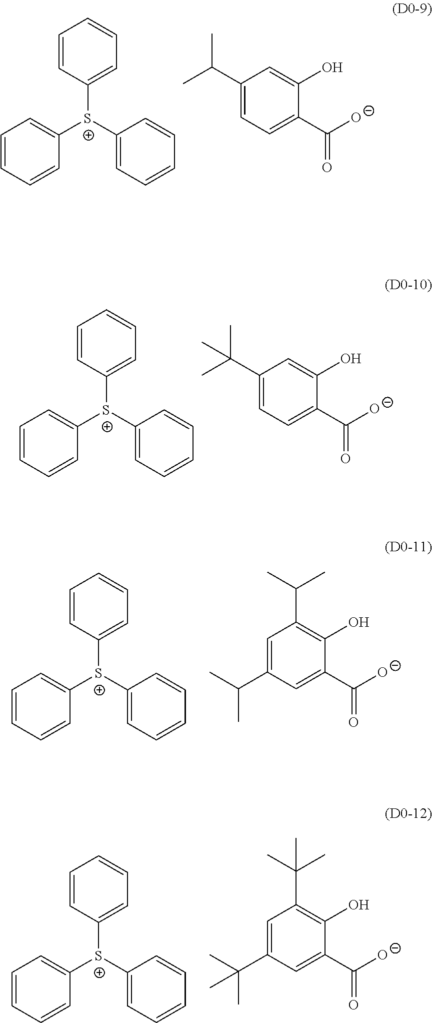

Compound (D0-9): ¹H-NMR (DMSO-d6) δ (ppm)=7.89-7.75 (m, 15H), 7.47 (d, 1H), 7.11 (dd, 1H), 6.84 (d, 1H), 2.99 (sept, 1H), 1.20 (d, 6H)

Compound (D0-10): ¹H-NMR (DMSO-d6) δ (ppm)=7.89-7.74 (m, 15H), 7.48 (d, 1H), 7.05 (dd, 1H), 6.88 (d, 1H), 1.32 (s, 9H)

Compound (0-11): ¹H-NMR (DMSO-d6) δ (ppm)=7.90-7.72 (m, 15H), 7.20 (d, 1H), 6.89 (d, 1H), 3.15 (sept, 1H), 2.73 (sept, 1H), 1.14 (d, 12H)

Compound (D0-12): ¹H-NMR (DMSO-d6) δ (ppm)=7.88-7.78 (m, 15H), 7.61 (d, 1H), 7.12 (d, 1H), 1.34 (s, 9H), 1.21 (s, 9H)

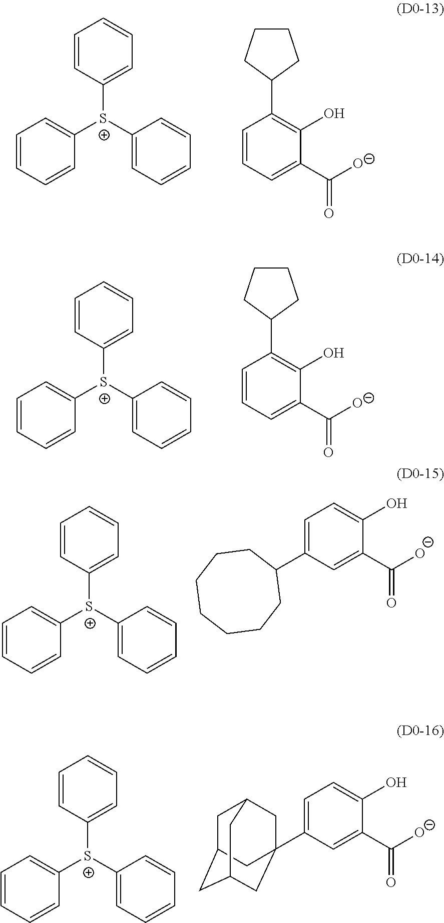

Compound (D0-13): ¹H-NMR (DMSO-d6) δ (ppm)=7.89-7.75 (m, 15H), 7.16 to 7.08 (m, 2H), 6.85 (t, 1H), 3.34 (m, 1H), 1.87 to 1.57 (m, 8H)

Compound (D0-14): ¹H-NMR (DMSO-d6) δ (ppm)=7.89-7.75 (m, 15H), 7.12 to 7.09 (m, 2H), 6.90 (t, 1H), 3.04 (tt, 1H), 1.66 to 1.27 (m, 10H)

Compound (D0-15): ¹H-NMR (DMSO-d6) δ (ppm)=7.89-7.75 (m, 15H), 7.32 (d, 1H), 6.88 (d, 1H), 6.62 (d, 1H), 3.02 (tt, 1H), 1.69 to 1.24 (m, 14H)

Compound (D0-16): ¹H-NM R (DMSO-d6) δ (ppm)=7.89-7.45 (m, 17H), 6.86 (d, 1H), 2.65-1.70 (m, 15H)

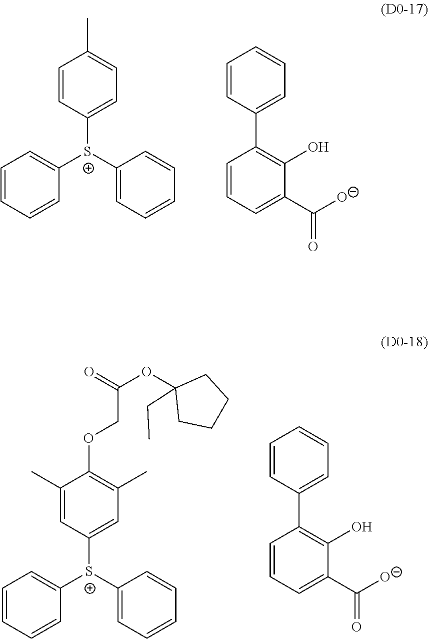 [SMILES: Cc1ccc([S+](c2ccccc2)c2ccccc2)cc1 ; O=C([O-])c1cccc(-c2ccccc2)c1O ; CCC1(OC(=O)COc2c(C)cc([S+](c3ccccc3)c3ccccc3)cc2C)CCCC1 ; O=C([O-])c1cccc(-c2ccccc2)c1O]

Compound (D0-17): ¹H-NMR (DMSO-d6) δ (ppm)=7.89-7.60 (m, 17H), 7.35 (d, 2H), 7.22 (d, 2H), 6.62 (m, 1H), 2.45 (s, 3H)

Compound (D0-18): ¹H-NMR (DMSO-d6) δ (ppm)=7.89-7.62 (m, 15H), 7.35 (d, 2H), 7.22 (d, 2H), 6.62 (m, 1H), 4.59 (s, 2H), 2.33 (s, 6H), 2.08-1.90 (m, 4H), 1.70-1.50 (m, 6H), 0.83 (t, 3H)

### <Preparation of Resist Composition>

### Examples 1 to 20 and Comparative Examples 1 to 5

The components listed in Tables 1 to 3 were mixed and dissolved to prepare a resist composition of each example (solid content concentration of 3.8% by mass).

TABLE 1

| | Com- | Com- | Com- | Com- | | | |
|---|---|---|---|---|---|---|---|
| | ponent | ponent | ponent | ponent | | | |
| | (A) | (B) | (D) | (F) | Component (S) | | |
| Example 1 | (A)-1 | (B)-1 | (D)-1 | (F)-1 | (S)-1 | (S)-2 | (S)-3 |
| | [100] | [10] | [5] | [4] | [1650] | [600] | [750] |
| Example 2 | (A)-1 | (B)-1 | (D)-2 | (F)-1 | (S)-1 | (S)-2 | (S)-3 |
| | [100] | [10] | [5] | [4] | [1650] | [600] | [750] |
| Example 3 | (A)-1 | (B)-1 | (D)-3 | (F)-1 | (S)-1 | (S)-2 | (S)-3 |
| | [100] | [10] | [5] | [4] | [1650] | [600] | [750] |
| Example 4 | (A)-1 | (B)-1 | (D)-4 | (F)-1 | (S)-1 | (S)-2 | (S)-3 |
| | [100] | [10] | [5] | [4] | [1650] | [600] | [750] |
| Example 5 | (A)-1 | (B)-1 | (D)-5 | (F)-1 | (S)-1 | (S)-2 | (S)-3 |
| | [100] | [10] | [5] | [4] | [1650] | [600] | [750] |
| Example 6 | (A)-1 | (B)-1 | (D)-6 | (F)-1 | (S)-1 | (S)-2 | (S)-3 |
| | [100] | [10] | [5] | [4] | [1650] | [600] | [750] |
| Example 7 | (A)-1 | (B)-1 | (D)-7 | (F)-1 | (S)-1 | (S)-2 | (S)-3 |
| | [100] | [10] | [5] | [4] | [1650] | [600] | [750] |
| Example 8 | (A)-1 | (B)-1 | (D)-8 | (F)-1 | (S)-1 | (S)-2 | (S)-3 |
| | [100] | [10] | [5] | [4] | [1650] | [600] | [750] |
| Example 9 | (A)-1 | (B)-1 | (D)-9 | (F)-1 | (S)-1 | (S)-2 | (S)-3 |
| | [100] | [10] | [5] | [4] | [1650] | [600] | [750] |
| Example 10 | (A)-1 | (B)-1 | (D)-10 | (F)-1 | (S)-1 | (S)-2 | (S)-3 |
| | [100] | [10] | [5] | [4] | [1650] | [600] | [750] |
| Example 11 | (A)-1 | (B)-1 | (D)-11 | (F)-1 | (S)-1 | (S)-2 | (S)-3 |
| | [100] | [10] | [5] | [4] | [1650] | [600] | [750] |
| Example 12 | (A)-1 | (B)-1 | (D)-12 | (F)-1 | (S)-1 | (S)-2 | (S)-3 |
| | [100] | [10] | [5] | [4] | [1650] | [600] | [750] |

TABLE 2

| | Com- | Com- | Com- | Com- | | | |
|---|---|---|---|---|---|---|---|
| | ponent | ponent | ponent | ponent | | | |
| | (A) | (B) | (D) | (F) | Component (S) | | |
| Example 13 | (A)-1 | (B)-1 | (D)-13 | (F)-1 | (S)-1 | (S)-2 | (S)-3 |
| | [100] | [10] | [5] | [4] | [1650] | [600] | [750] |
| Example 14 | (A)-1 | (B)-1 | (D)-14 | (F)-1 | (S)-1 | (S)-2 | (S)-3 |
| | [100] | [10] | [5] | [4] | [1650] | [600] | [750] |
| Example 15 | (A)-1 | (B)-1 | (D)-15 | (F)-1 | (S)-1 | (S)-2 | (S)-3 |
| | [100] | [10] | [5] | [4] | [1650] | [600] | [750] |
| Example 16 | (A)-1 | (B)-1 | (D)-16 | (F)-1 | (S)-1 | (S)-2 | (S)-3 |
| | [100] | [10] | [5] | [4] | [1650] | [600] | [750] |
| Example 17 | (A)-1 | (B)-1 | (D)-17 | (F)-1 | (3)-1 | (S)-2 | (S)-3 |
| | [100] | [10] | [5] | [4] | [1650] | [600] | [750] |
| Example 18 | (A)-1 | (B)-1 | (D)-18 | (F)-1 | (S)-1 | (S)-2 | (S)-3 |
| | [100] | [10] | [5] | [4] | [1650] | [600] | [750] |
| Example 19 | (A)-1 | (B)-2 | (D)-1 | (F)-1 | (S)-1 | (S)-2 | (S)-3 |
| | [100] | [10] | [5] | [4] | [1650] | [600] | [750] |
| Example 20 | (A)-1 | (B)-3 | (D)-1 | (F)-1 | (S)-1 | (S)-2 | (S)-3 |
| | [100] | [10] | [5] | [4] | [1650] | [600] | [750] |

TABLE 3

| | Com- | Com- | Com- | Com- | | | |
|---|---|---|---|---|---|---|---|
| | ponent | ponent | ponent | ponent | | | |
| | (A) | (B) | (D) | (F) | Component (S) | | |
| Comparative | (A)-1 | (B)-1 | (D)-19 | (F)-1 | (S)-1 | (S)-2 | (S)-3 |
| Example 1 | [100] | [10] | [5] | [4] | [1650] | [600] | [750] |
| Comparative | (A)-1 | (B)-1 | (D)-20 | (F)-1 | (S)-1 | (S)-2 | (S)-3 |
| Example 2 | [100] | [10] | [5] | [4] | [1650] | [600] | [750] |
| Comparative | (A)-1 | (B)-1 | (D)-21 | (F)-1 | (S)-1 | (S)-2 | (S)-3 |
| Example 3 | [100] | [10] | [5] | [4] | [1650] | [600] | [750] |
| Comparative | (A)-1 | (B)-1 | (D)-22 | (F)-1 | (S)-1 | (S)-2 | (S)-3 |
| Example 4 | [100] | [10] | [5] | [4] | [1650] | [600] | [750] |
| Comparative | (A)-1 | (B)-1 | (D)-23 | (F)-1 | (S)-1 | (S)-2 | (S)-3 |
| Example 5 | [100] | [10] | [5] | [4] | [1650] | [600] | [750] |

In Tables 1 to 3, each abbreviation has the following meaning. The numerical values in the brackets are blending amounts (parts by mass).
(A)-1: A polymer compound represented by Chemical Formula (A-1). The weight-average molecular weight (Mw) in terms of standard polystyrene, determined by the GPC measurement, was 7000, and the polydispersity (Mw/Mn) was 1.60. The copolymerization composition ratio (the ratio (molar ratio) between constitutional units in the structural formula) is l/m=50/50.

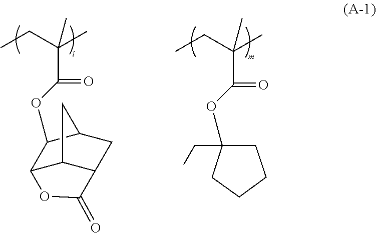

(B)-1: acid generator consisting of compound represented by Chemical Formula (B-1)
(B)-2: acid generator consisting of compound represented by Chemical Formula (B-2)
(B)-3: acid generator consisting of compound represented by Chemical Formula (B-3)

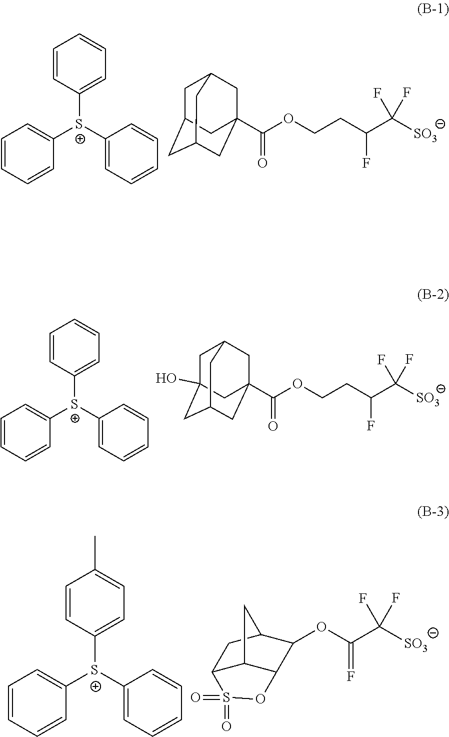

(D)-1 to (D)-18: photodecomposable bases formed of compounds represented by any of Chemical Formulae (D0-1) to (D0-18)
(D)-19: photodecomposable base formed of compound represented by Chemical Formula (D1-1)
(D)-20: photodecomposable base formed of compound represented by Chemical Formula (D1-2)
(D)-21: photodecomposable base formed of compound represented by Chemical Formula (D1-3)
(D)-22: photodecomposable base formed of compound represented by Chemical Formula (D1-4)
(D)-23: photodecomposable base formed of compound represented by Chemical Formula (D1-5)

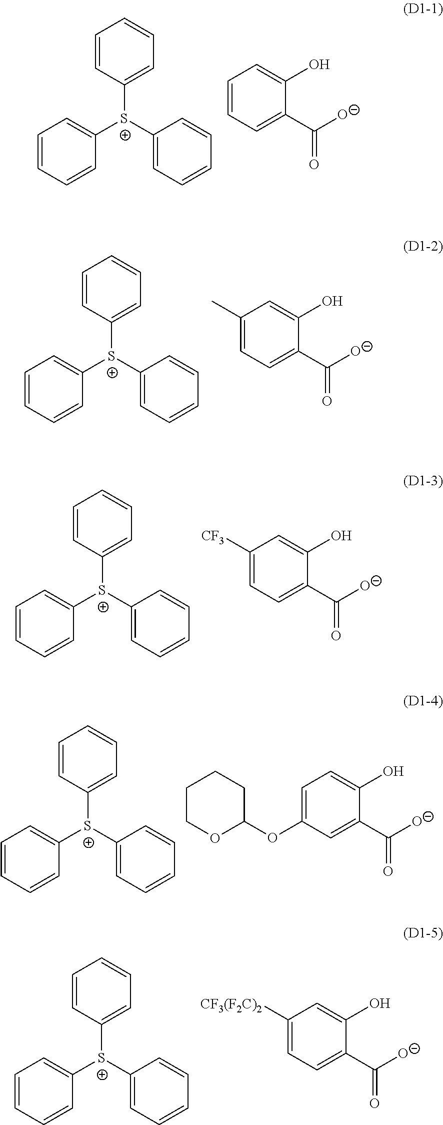

(F)-1: the fluorine-containing polymer compound represented by Chemical Formula (F-1). The weight-average molecular weight (Mw) in terms of the standard polystyrene equivalent value, determined by the GPC measurement, was 25,000, and the polydispersity (Mw/Mn) was 1.50. The copolymerization composition ratio (the ratio (the molar ratio) between constitutional units in the structural formula) is 1/m=80/20.
(S)-1: propylene glycol monomethyl ether acetate
(S)-2: propylene glycol monomethyl ether
(S)-3: cyclohexanone

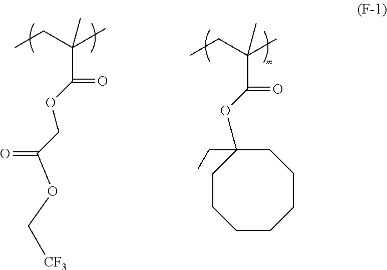

### <Resist Pattern Formation>

### Step of Forming Resist Film:

An organic antireflection film composition “ARC29A”, (manufactured by Brewer Science Inc.) was applied onto a 12-inch silicon wafer using a spinner and sintered and dried on a hot plate at 205° C. for 60 seconds to form an organic antireflection film having a film thickness of 98 nm.

The organic antireflection film was coated with the resist composition of each example using a spinner, subjected to a pre-bake (PAB) treatment at 100° C. for 60 seconds on a hot plate, and dried to form a resist film having a film thickness of 100 nm.

### Step of Performing Liquid Immersion Exposure on Resist Film:

Next, the resist film was selectively irradiated with an ArF excimer laser (193 nm) through a photomask (halftone: 6%) using an ArF exposure apparatus for liquid immersion XT 1900Gi [manufactured by ASML; numerical aperture (NA)=1.35, Annular, Sigma 0.97/0.78, Y deflection, liquid immersion medium: ultrapure water].

Thereafter, a post-exposure bake (PEB) treatment was performed at 100° C. for 60 seconds.

### Step of Developing Resist Film Exposed to Light to Form Resist Pattern:

Next, alkali development was carried out with a 2.38 mass % TMAH aqueous solution (trade name: NMD-3, manufactured by Tokyo Ohka Kogyo Co., Ltd.) at 23° C. for 15 seconds.

Thereafter, water rinsing was carried out for 15 seconds using pure water, followed by shake-off drying.

As a result, a line-and-space pattern (hereinafter, referred to as “LS pattern”) having a line width of 50 nm and a pitch of 100 nm (mask size of 50 nm) was formed in all the examples.

### [Evaluation of Line Width Roughness (LWR)]

In regard to the LS pattern, 3σ, which is a measure indicating LWR, was determined.

Here, “3σ” indicates triple values (3σ) (unit: nm) the standard deviation (a) determined from the measurement results obtained by measuring 400 sites of line positions in the longitudinal direction of the line using a scanning electron microscope (trade name: CG-5000, manufactured by Hitachi High-Tech Corporation, acceleration voltage of 500 V). The results are listed in the columns of “LWR (nm)” in Tables 4 to 6.

In a case where the value of the 3a is small, this indicates that the roughness of a line side wall is small and an LS pattern with a uniform width is obtained.

### [Evaluation of Water Mark Defect (WMD)]

In regard to the LS pattern, the number of total defects (total number of defects) in the wafer was measured using a surface defect observation device (product name: KLA2905, manufactured by KLA Corporation).

The measurement was performed 10 times for each sample, and the evaluation results of the average value are listed in the columns of “number of WMDs” in Tables 4 to 6. The target of the defect was limited to a defect having a size of 1 μm or greater.

TABLE 4

| | PAB | PEB | LWR | |
|---|---|---|---|---|
| | (° C.) | (° C.) | (nm) | Number of WMDs |
| Example 1 | 100 | 100 | 2.4 | 0.2 |
| Example 2 | 100 | 100 | 2.5 | 0.2 |
| Example 3 | 100 | 100 | 2.5 | 0.2 |
| Example 4 | 100 | 100 | 2.6 | 0.2 |
| Example 5 | 100 | 100 | 2.6 | 0.1 |
| Example 6 | 100 | 100 | 2.7 | 0.1 |
| Example 7 | 100 | 100 | 2.6 | 0.1 |
| Example 8 | 100 | 100 | 2.7 | 0.4 |
| Example 9 | 100 | 100 | 3.0 | 0.5 |
| Example 10 | 100 | 100 | 3.0 | 0.4 |
| Example 11 | 100 | 100 | 2.9 | 0.2 |
| Example 12 | 100 | 100 | 2.9 | 0.1 |

TABLE 5

| | PAB | PEB | LWR | |
|---|---|---|---|---|
| | (° C.) | (° C.) | (nm) | Number of WMDs |
| Example 13 | 100 | 100 | 2.8 | 0.4 |
| Example 14 | 100 | 100 | 2.8 | 0.4 |
| Example 15 | 100 | 100 | 2.8 | 0.3 |
| Example 16 | 100 | 100 | 2.8 | 0.3 |
| Example 17 | 100 | 100 | 2.4 | 0.1 |
| Example 18 | 100 | 100 | 2.5 | 0 |
| Example 19 | 100 | 100 | 2.5 | 0.2 |
| Example 20 | 100 | 100 | 2.4 | 0.3 |

TABLE 6

| | PAB | PEB | LWR | |
|---|---|---|---|---|
| | (° C.) | (° C.) | (nm) | Number of WMDs |
| Comparative | 100 | 100 | 3.1 | 4.5 |
| Example 1 | | | | |
| Comparative | 100 | 100 | 3.3 | 3.2 |
| Example 2 | | | | |
| Comparative | 100 | 100 | 3.4 | 3.5 |
| Example 3 | | | | |
| Comparative | 100 | 100 | 4.2 | 2.8 |
| Example 4 | | | | |
| Comparative | 100 | 100 | 4.6 | 0.7 |
| Example 5 | | | | |

As shown in the results listed in Tables 4 to 6, it was confirmed that the resist compositions of Examples 1 to 20 had both enhanced roughness characteristics and a high effect of suppressing occurrence of defects as compared with the resist compositions of Comparative Examples 1 to 5.

While preferred embodiments of the present invention have been described and illustrated above, it should be understood that these are exemplary of the present invention and are not to be considered as limiting. Additions, omissions, substitutions, and other modifications can be made without departing from the scope of the present invention. The present invention is not limited by the description above, but is limited only by the appended claims.

What is claimed is:

1. A resist composition which generates an acid upon light exposure and whose solubility in a developing solution is changed by an action of the acid, the resist composition comprising:
a base material component (A) whose solubility in a developing solution is changed by the action of the acid; and
a compound (D0) represented by General Formula (d0), 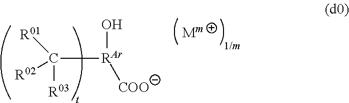
wherein R^{Ar} represents a (t+2)-valent aromatic hydrocarbon group which may have a substituent, t represents an integer of 1 or greater, R⁰¹ and R⁰² each independently represents a chain-like hydrocarbon group, R⁰³ represents a chain hydrocarbon group or a hydrogen atom, or two or more of R⁰¹, R⁰², and R⁰³ are bonded to each other to form a ring structure, where in a case where two of R⁰¹, R⁰², and R⁰³ are bonded to each other to form a ring structure, the ring structure is an alicyclic ring, and the remaining one represents a chain hydrocarbon group or a hydrogen atom, the chain hydrocarbon group may have a substituent, the ring structure may have a substituent, m represents an integer of 1 or greater, and M^{m+} represents an m-valent organic cation.

2. The resist composition according to claim 1, wherein the compound (D0) is a compound in which two or more of R⁰¹, R⁰², and R⁰³ in General Formula (d0) are bonded to each other to form a ring structure when two of R⁰¹, R⁰², and R⁰³ are bonded to each other to form a ring structure, the ring structure is an alicyclic ring, the remaining one represents a chain hydrocarbon group or a hydrogen atom, the ring structure may have a substituent, and the chain hydrocarbon group may have a substituent.

3. The resist composition according to claim 1, wherein the compound (D0) is a compound in which R⁰¹ and R⁰² in General Formula (d0) each independently represent a chain hydrocarbon group, and R⁰³ represents a chain hydrocarbon group or a hydrogen atom, wherein the chain hydrocarbon group may have a substituent.

4. The resist composition according to claim 1, further comprising an acid generator component (B) which generates an acid upon light exposure.

5. A resist pattern forming method comprising:
forming a resist film on a support using the resist composition according to claim 1;
performing liquid immersion exposure on the resist film; and
developing the resist film exposed to light to form a resist pattern.

6. A compound which is represented by General Formula (d0), 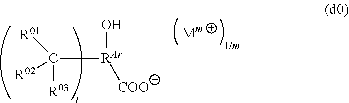
wherein R^{Ar} represents a (t+2)-valent aromatic hydrocarbon group which may have a substituent, t represents an integer of 1 or greater, R⁰¹ and R⁰² each independently represent a chain hydrocarbon group, R⁰³ represents a chain hydrocarbon group or a hydrogen atom, or two or more of R⁰¹, R⁰², and R⁰³ are bonded to each other to form a ring structure, where in a case where two of R⁰¹, R⁰², and R⁰³ are bonded to each other to form a ring structure, the ring structure is an alicyclic ring, and the remaining one represents a chain hydrocarbon group or a hydrogen atom, the chain hydrocarbon group may have a substituent, the ring structure may have a substituent, m represents an integer of 1 or greater, and M^{m+} represents an m-valent organic cation.

7. The compound represented by claim 6, wherein the compound is a compound in which two or more of R⁰¹, R⁰², and R⁰³ in General Formula (d0) are bonded to each other to form a ring structure, provided that when two of R⁰¹, R⁰², and R⁰³ are bonded to each other to form a ring structure, the ring structure is an alicyclic ring, the remaining one represents a chain-like hydrocarbon group or a hydrogen atom, the ring structure may have a substituent, and the chain-like hydrocarbon group may have a substituent.

8. The compound according to claim 6, wherein the compound is a compound in which R⁰¹ and R⁰² in General Formula (d0) each independently represents a chain hydrocarbon group, and R⁰³ represents a chain hydrocarbon group or a hydrogen atom, wherein the chain-like hydrocarbon group may have a substituent.
